(19) United States
(12) Patent Application Publication
(10) Pub. No.: US 2014/0193436 A1
(43) Pub. Date: Jul. 10, 2014

(54) EXTRACELLULAR TARGETED DRUG CONJUGATES

(71) Applicant: James R. Prudent, Madison, WI (US)

(72) Inventor: James R. Prudent, Madison, WI (US)

(73) Assignee: CENTROSE, LLC, Madison, WI (US)

(21) Appl. No.: 14/128,429

(22) PCT Filed: Jun. 25, 2012
(86) PCT No.: PCT/US12/44029

§ 371 Date: Jan. 23, 2014

Related U.S. Application Data
(60) Provisional application No. 61/500,756, filed on Jun. 24, 2011.
(60) Provisional application No. 61/507,882, filed on Jul. 14, 2011.
(60) Provisional application No. 61/551,287, filed on Oct. 25, 2011.

(51) Int. Cl. A61K 47/48; A61K 31/7048; G01N 33/569

(52) U.S. Cl. CPC A61K 47/48561; G01N 33/56966; A61K 31/7048
USPC 424/178.1; 530/391.7; 435/7.21

(57) ABSTRACT

Extracellular-targeted drug conjugates (EDC) in which a targeting moiety targeting a protein associated with the Na,K-ATPase is linked to a drug that interacts with the Na,K-ATPase through a linker a stable linker are useful in the treatment of disease and as tools for the evaluation of biological systems.

### BACKGROUND

1. Field of the Invention

The present invention provides drug conjugates in which an antibody or other targeting agent (e.g. a targeting moiety) that targets an extracellular target that is not a Na,K-ATPase is linked through a linker to a drug that targets the Na,K-ATPase. These conjugates are useful in the treatment of disease and also as tools for the evaluation of biological systems. The invention relates to the fields of biology, chemistry, medicinal chemistry, medicine, molecular biology, and pharmacology.

2. Description of Related Disclosures

All fundamental biological processes, including development, immunity, and tumorigenesis, are related to the selective and differential expression of genes in different tissues and cell types. For example, the formation of many malignant tumors has been shown to be associated with the production and/or expression of certain specific cell surface signaling molecules. One of the goals of modern molecular medicine is to find ways to target drugs selectively to reduce or eliminate the drug's off target toxic effects. Delivering drugs to a specific target that is unique to or expressed at higher levels in diseased cells types using targeting moieties such as antibodies, peptides or aptamers has been investigated. Attaching these targeting moieties directly to the drug through linkers or to nanoparticles has also been investigated.

One such drug targeting system is termed, “antibody drug conjugates” or ADC for short has been studied intensively since 1985 (see, for example, U.S. patent publication No. 2009/0220529, incorporated herein by reference). Members of this class of targeted therapeutics are composed of an antibody specific to an antigen, a drug or drugs that act intracellularly, and a linker that connects the antibody to the drug(s). Though there have been a few examples where peptide drugs attached to nonbinding antibodies have external cellular activity, ADC activity is typically realized only if some sort of membrane penetration by the drug occurs via either extracellular or intercellular drug release from the conjugate (U.S. Pat. No. 7,521,425).

Recently, however, an exciting new development related to ADC technology has emerged. In this approach, the targeting moiety, which can be an antibody, is attached to the drug via a non-cleavable or other stable linker (see PCT Pat. Pub. No. 2011/031870, incorporated herein by reference). This new class of ADCs, termed “Extracellular-targeted Drug Conjugates” or “EDC” includes EDC in which the antibody or other targeting moiety is targeted to the Na,K-ATPase (including any of the alpha, beta, or gamma subunits, but in a number of embodiments targeting dysadherin, the subunit gamma 5) and linked to a drug that binds the active site on the alpha subunit of the Na,K-ATPase, such as a member of the class of cardiac glycosides.

There remains a need for new EDC for the treatment of disease. The present invention meets this need. There also remains a need for methods and reagents to identify and evaluate protein-protein interactions between the Na,K-ATPase and cell signaling pathway proteins on the cell surface. The present invention also meets this need.

### SUMMARY OF THE INVENTION

The present invention generally relates to an extracellular-targeted drug conjugate (EDC) comprising a targeting moiety linked by a non-cleavable linker to a therapeutic agent, wherein the targeting moiety binds to an extracellular target that is not a Na,K-ATPase, and wherein the therapeutic agent acts on the Na,K-ATPase.

The present invention relates to the discovery that a wide variety of proteins interact with the Na,K-ATPase to modulate biochemical, e.g. cell signaling, pathways. The invention also relates to the discovery that, by targeting an EDC to an extracellular target that is not a Na,K-ATPase such as a cell surface signaling pathway protein that interacts with the Na,K-ATPase, one can modulate a wide variety of important cell signaling pathways. In accordance with the invention, EDC that contain a drug that binds to the Na,K-ATPase (e.g., at the alpha subunit or at another site on the Na,K-ATPase that perturbs or disrupts the interaction between the Na,K-ATPase and a cell surface signaling pathway protein) and a targeting moiety that targets the associated extracellular target that is not a Na,K-ATPase such as a cell surface signaling pathway protein are provided. Thus, the present invention relates to a new understanding of the important role of the Na,K-ATPase and provides new technology to deliver therapeutic agents that modulate the activity of the Na,K-ATPase selectively. Thus, in one aspect, the invention provides EDC targeted to specific cell surface complexes (e.g. to antigens, which may be on proteins, which may be receptors) that contain a extracellular target that is not a Na,K-ATPase such as a cell surface signaling pathway protein and the Na,K-ATPase.

The EDC of the invention also contain an agent that binds to or otherwise interacts with the Na,K-ATPase. The agent can be a drug, e.g. a therapeutic agent, if the EDC is to be used as a drug (such EDC are also useful as research tools). The agent can also be a diagnostic agent, such as a labeled cardiac glycoside, if the EDC is used for diagnosis (e.g., to determine if a particular patient is likely to respond to therapy or has a disease or condition amenable to treatment with the EDC) or as a research tool. In one embodiment, the drug interacts with the alpha subunit of the Na,K-ATPase and is conjugated to a targeting moiety that binds to a protein (other than the alpha or another subunit of the Na,K-ATPase) complexed to the Na,K-ATPase. Because the Na,K-ATPase is an extracellular protein, internalization of the EDC is not required, and the targeting moiety and the therapeutic (or diagnostic) agent work in concert to achieve the desired therapeutic (or diagnostic) effect.

Thus, in one aspect, the invention provides an EDC in which the therapeutic agent is covalently linked to a targeting moiety (e.g. an antibody) through a stable (and, in some embodiments, non-cleavable) linker that remains intact and uncleaved for the EDC to exert its maximal therapeutic effect. The targeting moiety (e.g. antibody) of the EDC targets an extracellular target that is not a Na,K-ATPase and that complexes with the Na,K-ATPase, and the target of the agent (e.g. drug or diagnostic agent) of the EDC is the Na,K-ATPase, including but not limited to the alpha subunit. In certain embodiments, the target of the agent is at a site on the Na,K-ATPase via which it is associated with the extracellular target that is not a Na,K-ATPase such as a cell surface signaling pathway protein that forms the complex with the Na,K-ATPase. In these embodiments, binding of the agent to the protein modulates (inhibits or activates) the interaction of the protein with the Na,K-ATPase. In many other embodiments, however, the agent targets the alpha subunit of the Na,K-ATPase. In many of these embodiments, the agent is an aglycon of a cardiac glycoside.

In various embodiments, the target for the targeting moiety (e.g. antibody) resides within a multi-protein complex that includes the Na,K-ATPase. In various embodiments, the targets of the agent and targeting moiety are different but in close proximity to one another (in the disease or other state of interest) such that the EDC only exerts its desired effect when its targeting moiety and agent interact with their respective targets simultaneously. Thus, the targeting moiety's target is distinct from the target of the therapeutic (or diagnostic) agent, but the two targets exist within close proximity such that the targeting moiety and agent act in concert or even synergistically with one another. Thus, the EDC of the invention is generally only therapeutically effective when both the targeting moiety's and agent's targets are in close proximity to one another on the cell, tissue, or organ to which the therapy is targeted.

In various embodiments, the targeting moiety of the EDC is an antibody. In one embodiment, the antibody is a bi-antibody in which one Fab is linked to the agent and the other Fab targets a protein associated with (e.g., modulates a biochemical pathway via interaction with) the Na,K-ATPase. In various embodiments, the agent is a drug or other agent that inhibits the activity of the Na,K-ATPase, including but not limited to drugs that are aglycons of cardiac glycosides. In other embodiments, the agent is a drug or other agent that modulates a signaling pathway mediated by the Na,K-ATPase by a means other than direct inhibition, including but not limited to drugs such as dimethyloxallyl glycine, glibenclamide, perillyl alcohol, statins and progesterone.

In one embodiment, the linker in the EDC of the invention is a non-cleavable linker, such as a linker comprised of polyethylene glycol (PEG) and one or more glycosides. In one embodiment of the EDC of the invention, a single agent is attached to a single targeting moiety through a stable or non-cleavable linker, and the targeting moiety and agent bind to their targets and/or act simultaneously or substantially simultaneously. In various embodiments, the invention provides sets of EDC that vary only by the length of the non-cleavable linker.

In another aspect, the present invention provides compositions including pharmaceutical formulations and unit dose forms and drug delivery systems useful in the treatment of disease. In one embodiment, the invention provides a composition that comprises, or alternatively consists or consists essentially of, as an active ingredient, an EDC of the invention. In one embodiment, the composition is a pharmaceutical formulation suitable for parenteral, including but not limited to intravenous, administration. In one embodiment, the invention provides pharmaceutical formulations that comprise, or alternatively consist or consist essentially of, an EDC of the invention in combination with a pharmaceutically acceptable vehicle, vector, diluent, and/or excipient. In other embodiments, the invention provides compositions that contain, in addition to an EDC of the invention, at least one other active pharmaceutical ingredient.

The pharmaceutical formulations of the invention can be used in vivo for preventive, ameliorative, and/or curative purposes for diseases or disorders. Non-limiting examples of diseases or disorders for which the pharmaceutical formulations according to the invention may be used include cancers, metastases, cellular apoptosis disorders, degenerative diseases, tissue ischemia, infectious diseases of a viral, bacterial or fungal nature, inflammation disorders, diabetes and pathological neo-angiogenesis. Thus, in accordance with the methods of the invention, a subject can be treated with a pharmaceutically effective amount of a compound or composition according to the invention. In one embodiment of the invention, the subject is a human subject. In other embodiments, the subject is a non-human mammal of veterinary or scientific research interest.

In another aspect, the invention provides methods for treating (or diagnosing or studying) a disease or other medical condition by administering to a patient in need of treatment a therapeutically effective dose of an EDC or other compound or pharmaceutical composition of the invention. For EDC to be used as therapeutic agents, the EDC exerts it greatest therapeutic effect only when both the targeting moiety and drug components are bound to their respective targets, and the therapeutic effect results from the EDC modulating a cell signaling pathway modulated by the Na,K-ATPase acting in concert with the other protein targeted by the EDC, e.g. a cell signaling pathway protein located on the surface of the cell in a complex with the Na,K-ATPase. Such EDC can also be used as diagnostic and/or research tools, e.g., the resulting modulation of the cell signaling pathway serves as a readout for an assay. Other EDC are useful in diagnosis and research, including EDC that complex with the Na,K-ATPase and a protein associated therewith but do not modulate the cell signaling pathway modulated by that association.

Thus, the methods, compounds, and compositions of the invention are generally useful for the treatments (and diagnosis and research) of medical conditions where therapeutic (or diagnostic) agents specific to extracellular targets that are not a Na,K-ATPase are administered. In various embodiments, the invention provides methods for treating or preventing (and diagnosing and studying) a disorder selected from the group consisting of cellular proliferative and/or differentiative disorders, disorders associated with bone metabolism, immune disorders, hematopoietic disorders, cardiovascular disorders, liver disorders, kidney disorders, muscular disorders, neurological disorders, hematological disorders, viral infections, pain, and metabolic disorders. In one embodiment, the invention provides methods for the treatment of cancer.

In another aspect, the invention provides novel cardiac glycosides or aglycon thereof that are useful in and of themselves as anti-cancer agents, and the invention also provides EDC comprising these cardiac glycosides or aglycons thereof, pharmaceutical compositions comprising these cardiac glycosides or aglycons thereof or EDC that comprise them, and methods for their manufacture and use.

In another aspect, the invention provides methods for the manufacture of the compounds, EDC, and compositions of the invention, as well as compounds useful in those methods. For example, the invention provides drug-linker agents that are useful in making EDC of the invention. These drug-linker agents comprise, in some embodiments, an aglycon of a cardiac glycoside, a non-cleavable linker comprising PEG and one or more glycosides, and a reactive group suitable for coupling to an antibody. The present invention also provides methods for the formulation and preparation of biological, pharmaceutical, cosmetic, and agricultural products, and for the use of the EDC compounds and compositions in those methods. In one embodiment, the invention pertains to uses of the compounds of the invention for the manufacture of a medicament for treating a disease.

In another aspect, the invention provides methods of treating a disease with an EDC of the invention either in monotherapy or in combination with one or more other therapeutics such that the combination acts to enhance or magnify one or more therapeutic effects, as compared to the use of either therapeutic alone.

In another aspect, the invention provides methods and reagents for detecting Na,K-ATPase interactions with an extracellular target that is not a Na,K-ATPase such as a cell surface signaling pathway protein co-located in a complex with the Na,K-ATPase. Generally, these methods employ an antibody-drug conjugate (ADC) in which the antibody targets a cell surface signaling pathway of interest and the drug targets the Na,K-ATPase and the two are linked by a stable or non-cleavable linker. This ADC together with appropriate controls (the antibody and the drug) are then tested in vitro and/or in vivo to determine if the ADC exerts a more potent and/or specific effect, such as cytotoxicity or inhibition of cell growth, proliferation, or differentiation, on one or more cell types of interest than either the antibody or drug alone. If the ADC is determined to exert a more potent or specific effect than either the antibody or drug alone, then the extracellular target (e.g. cell surface protein) targeted by the antibody is complexed with the Na,K-ATPase in such cell type(s) of interest.

Another aspect of the invention is an article of manufacture comprising an EDC; a container; and a package insert or label indicating how the EDC can be used to treat a disease or diagnose a condition or perform a research function.

In another aspect, the invention provides methods of binding to an extracellular target that is not a NaK-ATPase comprising contacting a cell expressing the target with an EDC as disclosed herein.

In another aspect, the invention provides methods of binding to an extracellular target that is not a NaK-ATPase comprising administering to a subject an amount of the EDC as disclosed herein effective to bind to the target.

These and other aspects and embodiments of the invention are described in detail below.

### DETAILED DESCRIPTION OF THE INVENTION

The present invention provides Extracellular-targeted Drug Conjugates or EDC in which the agent (a drug or diagnostic agent) and targeting moiety bind to or act on complexes containing the Na,K-ATPase (encoded by the ATP1 family of genes, including, for example the ATP1A1, ATP1A2, ATP1A3, and ATP1A4 genes). The EDC are useful in a variety of applications, particularly the treatment of human disease and other medical conditions. To facilitate an understanding of the invention, this detailed description is divided into sections. Section I provides definitions of terms used in this disclosure. Section II describes the Na,K-ATPase and its role in important cell signaling pathways. Section III describes antibodies and other targeting moieties useful in the EDC of the invention. Section IV describes linkers useful in the EDC of the invention. Section V describes therapeutic agents useful in the invention. Section VI describes particular embodiments of the EDC of the invention. Section VII describes pharmaceutical formulations of the inventions and methods for administering them to treat disease and other medical conditions. The detailed description of the invention is followed by a set of examples that illustrate useful methods and EDC of the invention.

PCT Pub. Nos. 2010/017480 and 2011/031870 and PCT App. No. US2012/028585, filed 9 Mar. 12, and all other patents, patent applications, and references from the scientific literature cited herein, are hereby incorporated by reference herein in their entireties.

### 1. Definitions

The term “aldehyde tag” or “ald-tag” is a peptide or peptidomimetic that contains an amino acid sequence derived from a sulfatase motif that is capable of being converted, or which has been converted, by action of a formylglycine generating enzyme (FGE) to contain a 2-formylglycine residue (referred to herein as “FGly”). The FGly residue generated by an FGE is often referred to in the literature as a “formylglycine”, although this is technically incorrect. Thus, “aldehyde tag”, as used herein, can refer to an amino acid sequence comprising an “unconverted” sulfatase motif (e.g., a sulfatase motif in which the cysteine or serine residues has not been converted to FGly by an FGE, but is capable of being converted) or to an amino acid sequence comprising a “converted” sulfatase motif (e.g., a sulfatase motif in which the cysteine or serine residues have been converted to FGly by action of an FGE).

The term “amino acid” refers to naturally occurring and non-natural amino acids, as well as amino acid analogs and amino acid mimetics. Naturally occurring amino acids are those encoded by the genetic code, as well as those amino acids that are not encoded by the genetic code and those that are modified form of encoded amino acids, e.g., beta-alanine, D-serine, hydroxyproline, gamma-carboxyglutamate, and O-phosphoserine. Amino acid analogs are compounds that have the same basic chemical structure as a naturally occurring amino acid, e.g., a carbon that is bound to a hydrogen, a carboxyl group, an amino group, or various R groups making non-naturally occurring amino acids (e.g., homoserine, norleucine, methionine sulfoxide, methionine methyl sulfonium). Such analogs have modified R groups (e.g., norleucine) or modified backbones, but retain the same basic chemical structure as a naturally occurring amino acid, e.g. beta amino acids, amino acids in D conformation. Amino acid mimetics refers to chemical compounds that have a structure that is different from the general chemical structure of an amino acid, but functions in a manner similar to a naturally occurring amino acid.

The term “antibody” refers to a protein or mixture of proteins that comprise one or more peptidic chains encoded by immunoglobulin genes or fragments thereof (including non-naturally occurring forms thereof produced by genetic engineering) that specifically bind and recognize an epitope of an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding. The antibodies comprise IgG (including IgG₁, IgG₂, IgG₃, and IgG₄), IgA (including IgA₁ and IgA₂), IgD, IgE, or IgM, and IgY. As used herein, the term “antibody” is meant to include whole antibodies, including single-chain antibodies, and antigen-binding fragments thereof. Antibodies can also be antigen binding antibody fragments and include, but are not limited to, Fab, Fab′ and F(ab′)₂, Fd, single-chain Fvs (scFv), single-chain antibodies, disulfide-linked Fvs (sdFv), diabodies, triabodies, tetrabodies, minibodies, and fragments comprising either a V_{L} or V_{H} domain, and Nanobodies (see PCT publication number WO 94/04678 and Nature Medicine, V9 (1) pp 129-134, 2003). An antibody can be from any animal origin including birds and mammals. Typically, antibodies in commercial or research use are human, murine, rabbit, goat, guinea pig, camelidae (e.g., camel, llamas) horse, or chicken antibodies. “Antibodies”, as used herein, includes monoclonal, immunoadsorbed polyclonal, chimeric, and humanized antibodies, as well as intact antibodies and isolated antibodies. Antibodies can be monospecific, bispecific, trispecific or greater multispecificity.

The term “antibody drug conjugate” or “ADC” refers to an antibody linked to a therapeutic agent (sometimes referred to herein as agent, drug, or active pharmaceutical ingredient) or agents.

The term “antigen” refers to the substance or target that an antibody or targeting moiety binds. An antigen is characterized by its ability to be “bound” by the antibody or targeting moiety. Antigen can also mean the substance used to elicit the production of targeting moieties, such as the production of antigen specific antibodies through immunizing with the antigen. An antigen is, in many embodiments, a protein, including but not limited to a receptor.

The term “antigen binding site” or “epitope” refers to the portion of the antigen to which a targeting moiety, such as an antibody, binds.

The term “aptamer” refers to a DNA, RNA, or oligonucleotide mimetic that is a targeting moiety and can be the functional equivalent of an antibody and specifically binds and recognizes an epitope of an antigen.

The term “specifically binds” refers to the ability of a targeting moeity to bind to an extracellular target with greater affinity than it binds to a non-target. In certain embodiments, specific binding refers to binding for an extracellular target with an affinity that is at least 10, 50, 100, 250, 500, or 1000 times greater than the affinity for a non-target.

The term “binding affinity” refers to the strength of interaction between an antibody (or other targeting moiety or drug or other agent) and its antigen (or target) as a function of its association and dissociation constants. Higher affinities typically mean that the targeting moiety has a fast on rate (association) and a slow off rate (dissociation). Binding affinities can change under various physiological conditions due to changes that occur to the antigen or antibody/targeting moiety under those conditions. Binding affinities of the targeting moiety can also change when therapeutic agents and/or linkers are attached. Binding affinities can also change when slight changes occur to the antigen, such as changes in the amino acid sequence or glycosylation of the antigen.

The term “cancer” refers to any of a number of diseases characterized by uncontrolled, abnormal proliferation of cells, the ability of affected cells to spread locally or through the bloodstream and lymphatic system to other parts of the body (e.g., metastasize), as well as any of a number of characteristic structural and/or molecular features. A “cancerous cell” or “cancer cell” is understood as a cell having specific structural properties, which can lack differentiation and be capable of invasion and metastasis. Examples of cancers are, breast, lung, brain, bone, liver, kidney, colon, and prostate cancer (see DeVita, V. et al. (eds.), 2005, Cancer Principles and Practice of Oncology, 6th. Ed., Lippincott Williams & Wilkins, Philadelphia, Pa., incorporated herein by reference in its entirety for all purposes).

The term “chimeric antibodies” refers to antibodies in which the Fc constant region of a monoclonal antibody from one species (typically a mouse) is replaced, using recombinant DNA techniques, with an Fc region from an antibody of another species (typically a human). For example, a cDNA encoding a murine monoclonal antibody is digested with a restriction enzyme selected specifically to remove the sequence encoding the Fc constant region, and the equivalent portion of a cDNA encoding a human Fc constant region is substituted. A CDR-grafted antibody is an antibody in which at least one CDR of a so-called “acceptor” antibody is replaced by a CDR “graft” from a so-called “donor” antibody possessing desirable antigen specificity. Generally the donor and acceptor antibodies are monoclonal antibodies from different species; typically the acceptor antibody is a human antibody (to minimize its antigenicity in a human), in which case the resulting CDR-grafted antibody is termed a “humanized” antibody. The graft may be of a single CDR (or even a portion of a single CDR) within a single V_{H} or V_{L} of the acceptor antibody, or can be of multiple CDRs (or portions thereof) within one or both of the V_{H} and V_{L}. Methods for generating CDR-grafted and humanized antibodies are taught by Queen et al. U.S. Pat. No. 5,585,089, U.S. Pat. No. 5,693,761 and U.S. Pat. No. 5,693,762; and Winter U.S. Pat. No. 5,225,539, which are incorporated herein by reference.

The term “circulatory structure” refers to body fluids, interstitial fluid, lymph and blood of a mammal, including tissues of the circulatory system.

The term “close proximity” refers to two targets X and Y that are in physical proximity such that, for example, when a targeting moiety (to X) and therapeutic agent (to Y) are conjugated through a linker, and both X and Y are bound to their respective targets, the conjugate induces a desired biological or medical response different from and superior to that induced by either X or Y alone. In one embodiment, the biological or medical response achieved is greater than that observed by either the targeting moiety or therapeutic agent alone. In another embodiment, the biological or medical response achieved is greater than that observed by the additive effects of the targeting moiety and therapeutic agent. For example, when X and Y are located on different molecules, but the molecules are present in the same multi-molecular complex, the targets are in “close proximity” as defined herein. In another example, when X and Y are on the same cell within 200 or fewer Angstroms from one another and act in concert to transmit a signal or otherwise generate a biochemical response, the targets are in “close proximity” to one another as defined herein. When X and Y are on different cells (and/or do not interact with one another), they are not in “close 87proximity” as defined herein.

The term “effective amount” refers to an amount of EDC, either alone or as a part of a pharmaceutical composition, that is capable of having any detectable, positive therapeutic effect on any symptom, aspect, parameter or characteristics of a disease state or condition when administered to a subject. Such effect need not be absolute to be beneficial.

The term “epitope” refers to groupings of molecules such as amino acid residues or sugar side chains at the surface of antigens that usually have specific three dimensional structural characteristics, as well as specific charge characteristics, and that are capable of specific binding by a monoclonal antibody.

The terms “extracellular” and “cell surface” refers to proteins, antigens, or epitopes located on the external portion of a cell membrane or in the fluids of the circulatory structure (for example, angiotensin converting enzyme is an extracellular protein).

The term “extracellular target” refers to a target that is not a Na,K-ATPase, such as a protein, ganglioside, antigen, and/or epitope located on the cell membrane or in the fluids of the circulatory structure. For example and without limitation, the following are extracellular targets: cell surface receptors, cell surface ion channels, CD (cluster of differentiation or designation) abbreviated proteins. More specifically, and again without limitation, the following are extracellular targets: CD147, CD44, CD98, CD87, CD230, CD56, CD71, MCTs, alpha-klotho, PE-NMT, fibroblast growth factor receptors, MMPs, c-MET, ATP-sensitive potassium channel and glutamate transporters. Generally, the targets for the targeting moiety and therapeutic agents of the EDC of the invention are both extracellular targets on the outer surface of the cell membrane. However, in some embodiments the therapeutic may bind to a target embedded in (for example an ion-channel blocker) the cell membrane. Targets that are not generally considered extracellular targets include, for example, chromosomal DNA, mRNA, tRNA, mTOR kinase, DNA and RNA polymerases, transcription factors, tubulin, and actin.

The term “extracellular-targeted drug conjugate” or “EDC” refers to a drug conjugate of the invention in which an antibody or other targeting moiety that targets an extracellular target is linked via a stable or non-cleavable linker to a drug or other agent that binds to an extracellular target.

The terms “FXYD5”, “dysadherin”, “ATPase subunit gamma 5”, or “gamma 5” are used interchangeably herein and refer to the gamma subunit 5 of the Na,K-ATPase ion pump complex.

The term “hetereobifunctional linker” refers to a linker with different reactive groups at either end, enabling sequential conjugation between two different functional groups in proteins and other molecules.

The term “intact antibody” comprises at least two heavy (H) chains and two light (L) chains inter-connected by disulfide bonds. Each heavy chain is comprised of a heavy chain variable region (abbreviated herein as HCVR or V_{H}) and a heavy chain constant region. The heavy chain constant region is comprised of three domains, CH₁, CH₂ CR) and CH₃. Each light chain is comprised of a light chain variable region (abbreviated herein as LCVR^{X} or V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxyl-terminus in the following order: FR1, CDR₁, FR₂, CDR₂, FR₃, CDR₃, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system. Examples of binding fragments include (i) a Fab fragment, a monovalent fragment consisting of the V_{L}, V_{H}, CL and CH₁ domains; (ii) a F(ab′)₂ fragment, a bivalent fragment comprising two Fab fragments linked by a disulfide bridge at the hinge region; (iii) a Fd fragment consisting of the V_{H} and CH₁ domains; (iv) a Fv fragment consisting of the V_{L} and V_{H} domains of a single arm of an antibody, (v) a dAb fragment (Ward et al., Nature 341: 544-546, 1989), which consists of a V_{H} domain; and (vi) an isolated complementarily determining region (CDR).

The term “linker” refers to a chemical moiety or bond that covalently attaches two or more molecules, such as a targeting moiety and a drug.

The terms “linker spacer group” and “spacer arm” refer to atoms in the linker that provide space between the two molecules joined by the linker.

The term “modified antibodies” refers to antibodies, such as monoclonal antibodies, chimeric antibodies, and humanized antibodies, which have been modified by, e.g., deleting, adding, or substituting portions of the antibody. For example, an antibody can be modified by deleting the constant region and replacing it with a constant region meant to increase half-life, e.g., serum half-life, stability or affinity of the antibody. Multiple molecules of a therapeutic agent or multiple different agents can be coupled to one antibody molecule. For example, different moieties can be coupled to an antibody molecule via the same linker, or multiple linkers that provide multiple sites for attachment (e.g., dendrimers) can be used.

The term “modulate” refers to an interaction of EDC with an extracellular target and the Na,K-ATPase so as to alter, either directly or indirectly, a cell signaling pathway including, for example, to limit or reduce (e.g., inhibit) or increase the activity of the cell signaling pathway.

The term “monoclonal antibody” refers to a preparation of antibody molecules of single molecular composition. A monoclonal antibody composition displays a single binding specificity and affinity for a particular epitope. The term “human monoclonal antibody” refers to antibodies displaying a single binding specificity which have variable and constant regions (if present) derived from human germline immunoglobulin sequences. Human monoclonal antibodies can be produced by a hybridoma which includes a B cell obtained from a transgenic non-human animal, e.g., a transgenic mouse, having a genome comprising a human heavy chain transgene and a light chain transgene fused to an immortalized cell, although the term “monoclonal antibody” is not limited to antibodies produced through hybridoma technology. The term “monoclonal antibody” refers to an antibody that is derived from a single clone, including any eukaryotic, prokaryotic, or phage clone, and not the method by which it is produced. Monoclonal antibodies can be prepared using a wide variety of techniques known in the art including the use of hybridoma, recombinant, and phage display technology.

The term “non-cleavable linker” refers to a stable linker that has the property of being more stable in vivo than either the therapeutic or the targeting moiety under the same physiological conditions. Examples of non-cleavable linkers include linkers that contain polyethylene glycol chains or polyethylene chains that are not acid or base sensitive (such as hydrazone containing linkers), are not sensitive to reducing or oxidizing agents (such as those containing disulfide linkages), and are not sensitive to enzymes that may be found in cells or circulatory system. Specific examples of non-cleavable linkers include SMCC linker (US Patent Application Pub. No. US20090202536). For illustrative purposes, examples of cleavable linkers include linkers that contain non-hindered glutathione sensitive disulfides, esters, peptide sequences sensitive to the peptidases such as cathepsin or plasmin, pH sensitive hydrazones [see Bioconjugate Chem., 2010, 21 (1), pp 5-13]. Specific examples of cleavable linkers include non-hindered disulfide linker SPP (US20090202536). In various embodiments, a non-cleavable linker has one or more of the following properties that can be readily characterized experimentally: (1) the non-cleavable linker remains relatively intact keeping the therapeutic agent attached to the targeting moiety for extended periods of time (e.g., between at least about 2 to 8 hours, or at least 1 to 5 days, or at least 5 to about 30 days) under physiological conditions; (2) the non-cleavable linker is stable to enzymes, e.g. in the circulatory structure; (3) the non-cleavable linker allows the EDC to maintain activity even after it has acted on a target on a cell; (4) the non-cleavable linker does not negatively interfere with the binding activity or specificity of the targeting moiety; and/or (5) the non-cleavable linker does not negatively interfere with the activity of the therapeutic agent. Attachment of a stable or non-cleavable linker may have an effect on a therapeutic agent; for example, the cytotoxicity of a cytotoxic agent may be decreased (but, in the EDC of the invention, is not eliminated) by linker attachment. Any decrease in activity caused by linker attachment, however, is more than offset by an increased therapeutic efficacy of the EDC comprising the linker and agent. Thus, the agent attached to the targeting moiety via the non-cleavable or stable linker displays benefits over the agent alone. Such benefits may include solubility, lower toxicity, improved pharmacokinetics, and/or increased therapeutic efficacy.

The terms “non-cleaved” and “uncleaved” refer to an EDC composition at any point in time in which the majority (for example, >50%, >60%, >70% or >80%) of EDC components present are intact, e.g., the linker used to attach the agent to the targeting moieties has not been cleaved.

The terms “non-internalizing targeting moiety” or “non-internalizing antibody” refer to a targeting moiety or antibody, respectively, that has the property of reacting (binding) under physiological conditions (at 37° C. and pH 7) in vivo or in vitro, to antigens outside of a cell, within the circulatory structure, or on a cell surface, and that, when bound to its target antigen, does not enter the cell and become degraded in the lysosome (see Cancer Res 2009; 69(6) 2358-64). In this context, “internalizing” and “internalization” refer to the process by which materials enter cells via lysosomal compartments. In one embodiment, the targeting moiety or antibody, when bound to its target antigen, does not enter the cell and become internalized in an endosome. The target of a “non-internalizing targeting moiety” or “non-internalizing antibody” is referred to herein as a “non-internalizing target,” which is a target that does not get internalized into the lysosome as a result of binding to a targeting moiety or antibody. Non-internalizing targets may, however, become internalized into the cell in other biological processes. Examples of non-internalizing targets include, but are not limited to CD20, CD21, and CD72. For illustrative purposes, “internalizing targets” include, for example and without limitation, CD79, and CD22.

The term “non-internalizing therapeutic agent” refers to a therapeutic agent (drug) that has the property of reacting in physiological conditions (at 37° C. and pH 7) in vivo or in vitro, with its target (typically, via binding to its receptor) without being internalized into cells.

The terms “peptide”, “polypeptide”, peptidomimetic and “protein” are used, somewhat interchangeably, to refer to a polymer of amino acid residues. The terms apply to amino acid polymers in which one or more amino acid residues is an artificial chemical mimetic of a corresponding naturally occurring amino acid, as well as to naturally occurring amino acid polymers. These terms also encompass the term “antibody”. “Peptide” is often used to refer to polymers of fewer amino acid residues than “polypeptides” or “proteins”. A protein can contain two or more polypeptides, which may be the same or different from one another.

The terms “pharmaceutically effective amount” and “effective amount” in the context of an amount of drug delivered refer to an amount of a drug that can induce a desired biological or medical response in a tissue, system, animal, or human.

The term “polyclonal antibody” refers to a preparation of more than one (two or more) different antibodies to an antigen. Such a preparation includes antibodies binding to a range of different antigen binding sites.

The term “receptor” refers to an extracellular target protein molecule, embedded in either the plasma membrane or the cytoplasm of a cell, to which one or more specific kinds of signaling molecules may bind. Each cell typically has many receptors, of many different kinds.

The term “stable in the circulatory structure” refers to the property of a compound, such as an EDC, to resist degradation and means that, for example, less than about 50%, or less than about 20%, or typically less than about 2%, of the compound is degraded or cleaved in the circulating blood at about 37° C. for at least about 2 hours.

The term “stable linker” refers to a linker that remains stable and intact until the conjugate has been delivered or transported to the target site—a stable linker remains covalently attached to the two molecules it links—in physiological conditions (at 37° C. and pH 7) in vivo or in vitro for a period of time sufficient to allow the EDC to reach the target(s) and bind to the target(s). Thus, a stable linker is generally stable within the circulatory structure (generally means below 5% degradation after at least a 2 hour period and, in some embodiments, at least 4, 8, 16, or 24 hour periods). A stable linker maybe cleaved by enzymes or physiological conditions (such as differing pH's) inside a cell, tissue, or organ. Examples of “stable” linkers include non-cleavable linkers, but stable linkers can be cleavable, so long as they generally aren't cleaved in vivo prior to the EDC reaching and binding to its target(s). For example, stable linkers can contain hindered glutathione sensitive disulfides, peptide sequences sensitive to the peptidases such as cathepsin, or pH sensitive hydrazones [see Bioconjugate Chem., 2010, 21 (1), pp 5-13 and Clin. Cancer Res. 2005 11(2 Pt 1):843-52]. Thus a stable linker can be a cleavable linker but only if the linker is not cleaved prior to the EDC that contains such linker reaching its target(s). For example, a cathepsin cleavable linker is a stable linker, because cathepsin is only found in the lysosome, which is intracellular. Examples of unstable linkers are linkers that contain ester or acyl hydrazone linkages.

The term “substantially simultaneously” refers to two or more events that occur at the same time or within a relatively narrow time frame. In various embodiments, substantially simultaneously refers to two or more events that occur within about 60, about 40, about 30, about 20, about 10, about 5, about 2 or about 1 second or less than about one second of each other. For example, EDC of the invention have properties such that targeting moiety binding and agent (drug) action happen substantially simultaneously.

The term “synergistically” refers to an effect of two or more agents when used in combination that is greater than the sum of the effects of both agents when used alone. For example, in the EDC of the invention, the combined therapeutic effects of the interaction of the targeting moiety and the agent (drug) when linked through a linker are greater than the combined individual effects of the targeting moiety and agent when used alone. “Effects” can refer either to binding, therapeutic effect, and/or specificity.

The term “target” refers to the protein, glycoprotein, antigen, carbohydrate or nucleic acid to which a targeting moiety binds and also refers to the protein, glycoprotein, antigen, carbohydrate or nucleic acid to which a therapeutic agent binds. The agent and targeting moiety may bind to different targets in a “target complex”, where “target complex” refers to two or more molecules, such as the different subunits of a multi-subunit protein or two different proteins in a multi-protein complex, that are in close physical proximity with one another in vivo.

The term “target cells” refers to the cells that are involved in a pathology and so are preferred targets for therapeutic activity. Target cells can be, for example and without limitation, one or more of the cells of the following groups: primary or secondary tumor cells (the metastases), stromal cells of primary or secondary tumors, neoangiogenic endothelial cells of tumors or tumor metastases, macrophages, monocytes, polymorphonuclear leukocytes and lymphocytes, and polynuclear agents infiltrating the tumors and the tumor metastases.

The interchangeable terms “targeting moiety” and “targeting agent” refer to an antibody, aptamer, peptide, or other substance that binds specifically to a target. A targeting moiety may be an antibody targeting moiety (e.g. antibodies or fragments thereof) or a non-antibody targeting moiety (e.g. aptamers, peptides, or other substances that bind specifically to a target).

The term “target tissue” refers to target cells (e.g., tumor cells) and cells in the environment of the target cells.

The terms “therapeutic agent” and “drug” and “agent” are used interchangeably herein to refer to a compound that, when present in a therapeutically effective amount, upon binding to a site of action, produces a therapeutic effect, and whose site of action is located or whose effect will be exerted on the surface or inside target cells. By way of example, a therapeutic agent may be a chemical agent, such as an antibiotic or anti-cancer agent, a polypeptide, a protein, or a nucleic acid.

The term “therapeutic effect” refers to the reduction, elimination, and/or prevention of a disease, symptoms of the disease, or side effects of a disease in a subject.

The term “to increase the half-life time” means to increase the mean residence time of a compound, typically a therapeutic agent, in the blood or to reduce the blood or plasmatic clearance compared to a reference compound.

The terms “treating” and “treatment” are used interchangeably to refer to the administration of a therapeutic agent or composition to a patient who has a disease or disorder (e.g., cancer or metastatic cancer), a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease. “Treating” or “treatment” of cancer or metastatic cancer refers to the treatment or amelioration or prevention of a cancer, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters, including the results of an examination by a physician. Accordingly, the term “treating” includes the administration of a therapeutic agent to prevent or delay, to alleviate, or to arrest or inhibit development of the symptoms or conditions associated with a disease, including but not limited to neoplastic disease.

The term “tumor specific antigen” refers to proteins or other molecules that are unique to a tumor or is at least more abundant on tumor cells, relative to normal cells.

### II. Na,K-ATPase and Cell Signaling Pathways

The Na,K-ATPase functions as an ion channel and a signal transducer. The Na,K-ATPase is an integral transmembrane protein enzyme that initially was only thought to import potassium ions and export sodium ions against a concentration gradient but more recently has been shown to also transmit signals across the cell membrane. The enzyme is made of three subunits: the alpha, which is the catalytic core and the main target for steroidal compounds; the beta subunit, which is believed to traffic the alpha subunit to specific cell surface locations and is required for alpha subunit activity; and the gamma subunit, which is an auxiliary subunit and which exists in a variety of cell type specific isotypes. The main binding site of cardiac (cardioactive) glycosides to the Na,K-ATPase is located in a cavity formed by the transmembrane helices M1, M2, M4, M5, and M6 [Proc. Natl. Acad. Sci., 2009, 106, 13742-13747].

The binding of cardiac glycosides to the Na,K-ATPase has been reported to trigger many activities against a variety of different diseases. For example, this binding event has been reported to inhibit HIF-1 alpha synthesis, interleukin 8 (IL-8) production and hypoxia inducible factor NF-kB, as well as to have activity relevant to the pathogenesis and therapy of chronic heart and kidney failure, cardiac hypertrophy and arrhythmias, atherosclerosis, diabetes, neurological diseases and cancer [Current Medicinal Chemistry, 2011, 18, 872-885]. Other than the currently approved uses for cardiac glycosides (e.g. atrial fibrillation, atrial flutter and heart failure), a number of cardiac glycosides and extracts containing cardiac glycosides have been or are currently being tested in clinical trials for indications such as cystic fibrosis, cancer, blood pressure, and rheumatoid arthritis [respective http://clinicaltrials.gov/ designations: NCT00782288, NCT00837239, NCT00852787, NCT01355354].

Targeted drugs including, steroidal drugs, have benefits in the treatment of diseases. For example, IL-8 (a potent neutrophil recruiting and activating factor) is typically detected in clinical samples from patients with many inflammatory lung diseases, including the acute respiratory distress syndrome, chronic obstructive pulmonary disease and asthma. This has led clinicians to believe that antagonism of IL-8 may be a practicable therapeutic strategy for disease management [American J Respiratory Medicine. 1 (1), 19-25 (2002)]. Specifically in the case of cystic fibrosis, profound lung inflammation that characterizes the disease is mainly attributed to an overproduction of IL-8 in the lung. Cardiac glycoside Na,K-ATPase binding has been shown to inhibit IL-8-mediated biological responses in diverse cell types by modulating IL-8 receptors through altering membrane fluidity and microviscosity [J. Cell Physiol. 207, 195-207 (2006)]. In addition, low concentrations of cardiac glycosides trigger downstream signaling cascades that prevent cell death and induce proliferation, which are relevant in the context of ischaemic stroke [Proc. Natl Acad. Sci. USA 103, 10461-10466 (2006)]. This binding event has also been studied in the context of cell signaling pathways relevant to neurodegenerative diseases such as spinobulbar muscular atrophy and other polyglutamine-related diseases [Hum. Mol. Genet. 13, 437-446 (2004)].

There are, however, major concerns with using Na,K-ATPase directed therapeutics. First, Na,K-ATPases are found on all cells, and drugs that target them have the potential to effect a wide range of biological processes. For example, elevated levels of endogenous glycosides are associated with high blood pressure and hypertension [Proc. Natl. Acad. Sci. USA 102, 15723-15724 (2005) and Proc. Natl Acad. Sci. USA 102, 15845-15850 (2005)]. Secondly, at levels required to show anti-cancer efficacy, most drugs tested to date that target the Na,K-ATPase have been shown to be too toxic. This toxicity can include cardiac toxicity and/or neurotoxicity. Thirdly, the therapeutic window of cardiac glycosides is small. In fact, the approved cardiac glycosides (e.g. digoxin or proscillaridin) can cause death in patients at 2-3 times the level approved for administration Anesth Prog. 2007 Spring; 54(1): 19-24. These concerns greatly limit the use of drugs that act on the Na,K-ATPase.

In addition, previous reports of the effects of drugs that act on the Na,K-ATPase have been difficult to interpret. For example, even though it has been suggested that the Na,K-ATPase may regulate cholesterol [The Journal of Biological Chemistry, 2009, 284, 14881-14890], interact with Klotho (a transmembrane protein involved in the aging process) [FEBS Lett. 2011 Jun. 23; 585(12):1759-64], complex with n-methyl transferase [BMC Struct Biol. 2010 May 25; 10:12], and associate with CD147 [The Journal of Neuroscience, 2007, 27(45): 12331-12340], the reasons for and results of these interactions are not understood and have not been exploited for therapeutic or other useful purposes.

The present invention arises in part from the discovery that the Na,K-ATPase closely associates (complexes) with other cell surface proteins and acts in concert with them to modulate cell signaling pathways and that EDC targeting such complex have remarkable therapeutic activity, particularly in the treatment of cancer. The EDC of the invention overcome the prior problems associated with drugs targeting the Na,K-ATPase by ensuring that the drugs only act on Na,K-ATPase's that complex with the targeting moeity's target of the EDC and not on all Na,K-ATPases. This remarkable achievement arises from the use of a targeting moiety that directs a drug that acts on the Na,K-ATPase to act only on Na,K-ATPases associated with the targeting moiety's specific target—a cell surface protein involved in modulating a cell signaling pathway of interest. Targeting moieties useful in EDC of the invention are described in the following section.

III. Antibodies and other Targeting Moieties

The present invention provides EDC comprising a targeting moiety linked to a therapeutic agent via a stable or non-cleavable linker (e.g., a linker that has to be intact or non-cleaved for the EDC to exert its maximal therapeutic effect). These EDC act on complexes of the Na,K-ATPase and a protein that associates with the Na,K-ATPase. The EDC of the invention deliver therapeutic agents more selectively to target cells and tissues. In many embodiments, the EDC contains a targeting moiety that binds (e.g., specifically binds) to an extracellular target that is not a Na,K-ATPase and that associates with the Na,K-ATPase to modulate a cell signaling pathway and contains an agent that binds to the Na,K-ATPase (or to a protein binding site that blocks interaction with the Na,K-ATPase) that is attached to the targeting moiety via a stable or non-cleavable linker. Thus, in most embodiments, the targeting moiety and therapeutic agent bind to or act on different extracellular targets, e.g., the targeting moiety targets an extracellular target associated with the Na,K-ATPase and the agent targets the Na,K-ATPase. In many embodiments, the targeting moiety binds to a target in close proximity to the Na,K-ATPase that modulates a cell signaling pathway, e.g., it is a signaling pathway protein located on the cell surface, and the therapeutic (or diagnostic) agent acts on or binds to the Na,K-ATPase.

The targeting moiety of the EDC of the invention directs the EDC to the target cell or tissue that contains the target of the targeting moiety and the target of the therapeutic agent, e.g., in many embodiments, the Na,K-ATPase. Thus, in some embodiments, the therapeutic agent not only produces a desired therapeutic effect but also enhances the targeting properties of the EDC. In some embodiments, the targeting moiety and the agent work synergistically at directing the EDC to the target or targets. In some embodiments, the targeting moiety also has a therapeutic effect. In all embodiments, the stable or non-cleavable linker maintains the attachment of the targeting moiety to the therapeutic agent under physiological conditions for a sufficient period of time for the EDC to bind and exert a therapeutic effect.

The three portions of the EDC of the invention can thus comprise, consist essentially of or consist of: (1) a targeting moiety that binds to an extracellular target that is not a Na,K-ATPase and that is associated with and in close proximity to the Na,K-ATPase in the disease or other condition of interest; (2) a stable or non-cleavable linker that connects the targeting moiety to the therapeutic and remains intact (uncleaved) during the time needed for the EDC to bind to its target; and (3) a therapeutic (or diagnostic) agent that acts on or binds to the Na,K-ATPase (or to a site on an associated protein that controls association with the Na,K-ATPase).

In many embodiments of the EDC of the invention, the targeting moiety is a human antibody that does not induce internalization upon target binding and thus is not internalized into a lysosome once bound to its extracellular target.

Substantial efforts have been made to exploit antibodies to carry highly toxic payloads to infected or cancerous cells to bring and release drugs inside of cells thus acting like prodrugs. The “antibody-drug conjugate” or “ADC” approach is one such example. Other efforts to exploit the pharmacokinetic or dynamics of antibodies have lead to attaching drugs with poor drug qualities (e.g. short serum ½—life, or degradation) to antibodies. In this latter case, the drug does not require antibody release for activity and the antibody does not target the drug to a specific antigen. In some instances, the therapeutic agent is conjugated to the antibody's target binding site cancelling the antibody's target binding capabilities.

A first difficulty associated with the ADC approach has to do with cell penetration. To exert a therapeutic effect, an entire ADC or, at the least, the drug portion of an ADC must enter the target cell. Approaches to allow the ADC to enter the cell intact include targeting receptors that internalize the ADC or use of peptide-mediated membrane penetration to deliver antibodies to their intracellular target proteins (see US Pat. App. Pub. No. US20080063633). Many such receptors that have been found to become internalized upon antibody binding in one cell type may not be internalized in another, limiting the generality of the approach [see Cancer Res 2009; 69(6):2358-64]. In addition, as discussed below, internalization is typically intended to release the agent from the antibody. This release then allows free drug to traffic out of the cell and to interact with normal cells that neighbor the diseased cells, resulting in undesired toxicity. This phenomenon is termed the “bystander effect”. In addition, internalization must be followed by drug-antibody separation which is an inefficient process and thus the drug portion of the ADC requires extremely toxic drugs (Carter et.al. 2008 The Cancer Journal 14 (3) 154-169). The EDC of the invention do not require internalization and are typically not internalized, which alleviates many of the problems associated with the ADC approach.

A second difficulty associated with the ADC approach is that the drug of the ADC has to be released from the antibody before or after entering the cell to activate the drug or allow it entry into the cell. Approaches to allow selective release have included incorporating specific peptide sequences that are cleaved by cell specific peptidases (see US Pat. App. Pub. No. US20090220529) or contain environmentally sensitive linkages such that release or activation occurs near the cell membrane, within the cell membrane, or inside the cell cytosol or endosomal compartments. Certain cell types have been found to internalize the ADC into compartments that may not release the drug in active form, thus limiting the scope of the particular ADC [Cancer Res 2009; 69(6):2358-64]. The EDC of the invention do not require that the drug of the EDC be released from the targeting moiety, which alleviates many of the problems associated with ADC approaches that require drug-antibody separation.

A third difficulty associated with the ADC approach is that the ADC is a prodrug and therefore the drug portion of the ADC has to be activated when entering or coming into close proximity with the target cell. This requirement is important to ensure that release of the therapeutic agent from the antibody or activation of the agent portion of the ADC did not occur prior to the interaction of the ADC with their targets, which could lead to increased toxicity. Keeping the drug conjugated to the antibody is also important to maintain the efficacy of the ADC when not bound to its target cell and to keep unconjugated antibody from masking the target site from the active ADCs. The majority of publications on the ADC approach discuss methods to solve these problems. The EDC of the invention do not require activation to be effective (they are not prodrugs), which alleviates many of the problems associated with ADC approaches that require drug-antibody separation (drug activation).

A fourth difficulty associated with the ADC approach is that because the agents are released from the targeting moiety when internalized into the target cell, the agents are able to diffuse slowly out of the cell from which it was derived, and induce strong bystander activity on neighboring antigen-negative cells. Therefore, the free agent of the ADC becomes toxic to other normal (off-target) cells near the targeted cells, a phenomenon known as a bystander effect. This limits the dose at which the ADC can be administered. The EDC of the invention do not require the agent (drug) to release from the EDC to be effective, which alleviates problems associated with bystander effects and increases specificity.

A fifth difficulty associated with the ADC approach is lack of specificity: one but not both the antibody and agent might be specific to an extracellular target. Therefore, the specificity of the resulting ADC may not be as needed. ADCs are only as specific as the antibody's target. Many antibodies cannot be used to make safe and efficacious ADCs, because the antibody's target is also found on normal cells at sufficiently high levels. The EDC of the invention require two different targets for binding, and these targets are required to be within close proximity, which occurs less frequently, making EDC more specific.

In contrast to prior approaches, the present invention provides highly specific EDC that do not require cellular internalization, do not impose technically difficult constraints on the linker, are more specific, reduce bystander effects, and enhance the activity of the therapeutic agent by positioning the agent near its target site. The EDC of the invention therefore do not require that the targeting moiety facilitate internalization and do not rely on an antibody's non-targeting biological properties. The EDC of the invention can utilize targeting moieties other than antibodies, including but not limited to aptamers. The EDC of the invention require that the agent and the antibody remain intact for maximal specificity and activity. Because the antibodies or targeting moieties of the invention target extracellular antigens and internalization is not required for efficacy, lysosomal enzymatic degradation is not required. Because the linkers used in the EDC of the invention are stable or even non-cleavable, premature release of the drug from the EDC is minimized and linker design is more flexible and less complicated. Because the linker and therapeutic agent of the invention can be attached to targeting moieties for a variety of different targets in close proximity to the Na,K-ATPase, construction of new EDC is less complicated.

The EDC of the invention are more selective and/or less toxic than the drug they contain. In the EDC of the invention, the targeting moiety and/or linker can effectively prevent or dramatically reduce the therapeutic (and so reduce toxic, off-target) effect of the drug until the targeting moiety binds to its target. This is an especially important aspect of the invention, given the discovery that the Na,K-ATPase interacts with a myriad of other signaling proteins, creating the potential for significant, undesired “off-target” effects. Thus, the EDC of the invention are primarily active only when the targeting moiety is bound to its target and in close proximity to the therapeutic agent's target and when the EDC is intact. Taken together, these characteristics allow for more specific and less toxic EDC because the potential of acting on the Na,K-ATPase is only significantly high when the antibody binds to its target. The EDC of the invention are more selective, because both agent and antibody target sites need to be present, and in close proximity to one another, for the EDC to exert a therapeutic effect. The EDC of the invention are less toxic because the agent is linked through a stable linker to the targeting moiety that will selectively bind to its target, keeping the agent in close proximity and thus only able to act on Na,K-ATPases in that close proximity. Thus, the EDC of the invention are largely inactive when the targeting moiety is not bound to its target and when the Na,K-ATPase is not in close proximity to the target of the targeting moiety. Thus, the EDC of the invention are also largely inactive when the targeting moiety is bound to its target, but the targeting moiety's target is not in close proximity to the Na,K-ATPase. In effect, the linker prevents the drug from reaching the Na,K-ATPase unless it is in close proximity to the target of the targeting moiety.

The targeting moieties of the invention target antigens that associate with the Na,K-ATPase to modulate a cell signaling pathway. In view of this disclosure, those of skill in the art will now appreciate that there are numerous extracellular antigens known to be accessible to antibodies that associate with the Na,K-ATPase. Many of these are disease cell selective. Many of these antigens are found on cell surfaces. Many approved drugs act on extracellular targets on the cell surface. Illustrative examples of such targets, which may be exploited by the invention either as targets for targeting moieties (e.g. antibodies) for therapeutic agents include: CD147(Basigin), CD44 (HCELL), CD98 (LAT1), CD87(uPAR), CD230(PrP or prion-related protein), CD56 (NCAM), c-MET, RANK (receptor activator of NF-kB), CD71 (TfR1), CD166, EAATs, EpCAM, FGFR (fibroblast growth factor receptors), angiotensin receptor, ASCT2, calveolin, integrin family of proteins, GABAA receptor, GluR2, 5-HT1A receptor, IGF-1, InsP3R, Insulin Receptor, α-klotho, MCT1-4, mTNF alpha (transmembrane), OPG/RANKL, PE-NMT (N-methyltransferase), PLA2 (PC-phospholipase A2), RS1 (Retinoschisis), Sel-1, serotonin transporter, T-cell receptor, and TLR4.

An example of a targeting moiety useful in an EDC of the invention is one that specifically recognizes and binds CD147. CD147 is a pleiotropic molecule playing a role in fetal development, retinal function, and in T-cell maturation. It has been shown to be a cell-surface receptor for cyclophilins. It is expressed in areas of tissue remodeling: tumors, endometrium, placenta, skin and regions undergoing angiogenesis (see Iacono et al. 2007. Exp Mol. Path. 83:283-295) and stimulates matrix metalloproteinases (MMPs) and VEGF production. CD147 is induced upon monocyte differentiation and is expressed in human atheroma (Major T C, Liang L, Lu X, Rosebury W, Bocan T M. 2002. Arterioscler Thromb Vasc Biol. 22: 1200-1207). It has been shown that CD147 promotes invasion and metastasis in different tumor types via the induction of MMPs and the urokinase-type plasminogen activator system by peritumoral stromal cells. CD147 is also involved in angiogenesis, anoikis resistance, lactate efflux, multidrug resistance, and cell proliferation in cancer cells. CD147 overexpression and/or function has been associated with other pathological processes such as inflammatory responses, pulmonary fibrosis, rheumatoid arthritis, lupus erythematosus, heart failure, Alzheimer's disease and the infectivity cycle of the human immunodeficiency virus and coronaviruses in lymphocytes. (see Ruiz et al., J. Biol. Chem., Vol. 283, (9), 5554-5566, 2008). In addition, cleavage of CD147 and shedding of CD147 fragments may be involved in CD147 regulation or release of active fragments (Egawa et al. 2006 J Biol Chem 281(49): 37576-85).

Anti-CD147 antibodies have been reported. A murine antibody IgM Mab, CBL1 (Billings et al. Hybridoma 1:303-311, 1982, U.S. Pat. Nos. 5,330,896 and 5,643,740), was tested in steroid-refractory acute graft-versus-host disease (Heslop et al. The Lancet 346: 805-806; Deeg et al. 2001 Blood 98:2052-8). Human equivalent Mabs binding to epitopes overlapping that of CBL1 (aka ABX-CBL), near the transmembrane domain of the ECD were also developed (US2007048305A1). Koch et al. (Internat Immunol 11(5) 777-786, 1999) mapped CD147 epitopes associated with T- and B-cell activation, reporting that only the highest affinity monoclonal antibody (MEM-M6/6) of a group of antibodies made to CD147 was effective in preventing human T-cell activation and proliferation by the MAB against CD3, OKT3. A murine antibody to tumor cell derived human CD147, EIIF4 (Ellis, 1989 Cancer Res 49:3385-91; Biswas et al. Cancer Research 55, 434-439, 1995), demonstrated the ability to block lung carcinoma CD147 induced collagenase (matrix metalloproteinase-1 or MMP-1) activity from human fibroblasts. Binding of EIIF4 antibody to CD147 was shown to be abolished when a mutant ECD missing the N-terminal Ig domain was prepared (Biswas, C. et al., Cancer Res 55, 434-439, 1995). Ku et al. (Scan J Immunol 65(5) 435-443, 2007) identified MABs described as inhibitory for the CD147 associated MMP axis and, by using truncated CD147 sequences, identified key residues at the N-terminus for CD147 MMP induction activity.

Thus, while certain antibodies and other antagonists of CD147 are known, how the complex nature of the protein, including the two immunoglobulin domains, influences the myriad biological activities has not been thoroughly elucidated. Domain specific antagonists may prove to be useful therapeutic candidates for treating various of the pathologies associated with CD147 display and/or activation on various tissues. For example, therapeutic agents capable of blocking production MMPs or VEGF activity induced by CD147 could be advantageous in cancer therapy. Accordingly, there is a need to provide therapeutics that target human CD147 for use in therapy to diminish or eliminate symptoms of CD147-dependent diseases, as well as improvements over known antibodies or fragments thereof.

In one embodiment, the targeting moiety in the EDC is an antibody or other targeting moiety that specifically binds to CD147 encoded in humans by the BSG gene and/or to the extracellular domain of EMMPRIN (extracellular matrix metalloproteinase inducer; see US Pat. App. Pub. Nos. US20070048305 and US20060104974 and PCT Pub. No. 2010/036460). In one embodiment, the targeting moiety in the EDC is an antibody or other targeting moiety that binds to the extracellular domain of EMMPRIN and inhibits growth of tumor cells which over express EMMPRIN. In various embodiments, the targeting moiety of the EDC is an antibody that may be, for example, a monoclonal antibody, e.g. a murine monoclonal antibody, a chimeric antibody, a human antibody, or a humanized antibody. In one embodiment, the humanized antibody may be, for example, a humanized form of anti-CD147. In one embodiment, the humanized antibody may be ABX-CBL (a murine immunoglobulin M (IgM) monoclonal antibody that recognizes CD147 on the cell surface). In one embodiment, the antibody may be an antibody fragment, e.g. a Fab fragment. In one embodiment, the antibody of the EDC binds specifically to CD147.

Example 2 (see also Example 8) describes various illustrative EDC of the invention that target CD147. The data in the examples below shows that antibodies specific to CD147 can be used to produce EDC of the invention. The data show that each of the EDC prepared as described in Example 2 are more active than the control on cell lines expressing CD147 on their surface. EDC2.1 was determined to have 1.2 drugs per antibody on average, while the other EDC containing a targeting moiety specific to CD147 contain 5.9 to 8.6 drugs per antibody on average. The activity of EDC2.1 was least active of the four EDC tested. With the exception of EDC2.1, all EDC of Example 2 displayed sub-nanomolar activity, while the negative control (an antibody-drug conjugate containing an average of 7.4 drugs per antibody) displayed high nanomolar activity on all cell lines tested. Cancer cells tested included large-cell lung, colon, non-small-cell lung, pancreatic, breast, head and neck, small cell lung, melanoma, and myeloma.

Another example of a targeting moiety useful in making an EDC of the invention is one that specifically recognizes and binds to CD44. CD44 is a single-chain glycoprotein consisting of four functional domains: a conserved N terminal extracellular domain, a nonconserved membrane proximal region, a conserved transmembrane domain and a conserved cytoplasmic tail. CD44 ranges in molecular weight from 80 to 90 kDa and can generate close to 800 variant isoforms by differential alternative splicing (Cichy, J. et al., (2003) Journal of Cell Biology, 161:5, 839-843). The most common isoform of CD44, designated as standard CD44 (CD44s), is encoded by nine standard exons and has a molecular weight of approximately 90 kDa (Spring et al. 1988 Immunology 64 (1): 37-43). A variant form of CD44 (CD44v) contains additional exons, referred to as variant exons (v2-v10 in humans), that result in additional protein sequences in the extracellular and membrane proximal region of the protein. Cancers express variant isoforms of CD44. CD44 is ubiquitously expressed on many cell types, including leukocytes, fibroblasts, epithelial cells, keratinocytes and some endothelial cells, with CD44s being the most abundantly expressed isoform.

CD44 is known to participate in a wide variety of cellular functions, including cell-cell aggregation, retention of pericellular matrix, matrix-cell and cell-matrix signaling, receptor-mediated internalization/degradation of hyaluronan, and cell migration. Various functions of CD44 are dependent upon adhesive interactions with hyaluronan. CD44 is a cell surface glycoprotein receptor for the glycosamino glycan hyaluronan (HA). Hematopoietic cell E-selectin/L-selectin ligand (HCELL) is a glycoform of CD44 containing HECA-452 reactive sialylated, fucosylated N-glycans. HCELL is a ligand for both L-selectin and E-selectin (Dimitroff et al. Proc. Natl. Acad. Sci. USA, 97(25):13841-46, 2000; Dimitroff et al., J. Cell Biol., 153(6): 1277-1286, 2001). Some CD44 functions are affected or induced by post-translational modifications in the CD44 protein such as sulfation and/or glycosylations. Anti-CD44 antibodies that bind to specific variants of human CD44 with high affinity and specificity have been made (US Pat. App. Pub. No. US20050214283). The variants of CD44 glycoprotein impart metastasizing properties to a tumor whereas standard CD44 does not. Thus, variants of CD44 give tumors the ability of metastasizing through the lymphatic channels (Gunthert et al., Cell 65:13-24, 1991).

In one embodiment, the targeting moiety in the EDC is an antibody or other targeting moiety that specifically binds to the CD44 molecule (Indian blood group) encoded in humans by the CD44 gene (see U.S. Pat. No. 5,916,561 and US Pat. App. Pub. No. US20030103985). In one embodiment, the targeting moiety in the EDC is an antibody or other targeting moiety that specifically binds to a variant of the CD44 molecule (Wang et. al. 2009 Laryngoscope 119 (8): 1518-30 and Griffith et. al. 2010 Fertil. Steril. 93 (6): 1745-9). In one embodiment, the targeting moiety in an EDC of the invention is an antibody or other targeting moiety that specifically binds to the standard form of CD44 molecule. In one embodiment, the targeting moiety in the EDC is an antibody or other targeting moiety that binds to the extracellular domain of CD44 and inhibits growth of tumor cells which overexpress CD44. In various embodiments, the targeting moiety of the EDC is an antibody that may be, for example, a monoclonal antibody, e.g. a murine monoclonal antibody, a chimeric antibody, a human antibody, or a humanized antibody. In one embodiment, the humanized antibody may be Bivatuzumab (a humanized monoclonal antibody against CD44v6). In one embodiment, the antibody may be an antibody fragment, e.g. a Fab fragment. In one embodiment, the antibody of the EDC binds specifically to CD44. In one embodiment, the present invention relates to the use of antibodies directed against epitopes which are coded by the variant exons v4, v5, v6 and/or v9 or parts thereof.

Example 3 (see also Example 8) describes various illustrative EDC of the invention that target CD44. The data in the examples shows that antibodies specific to CD44 can be used to prepare EDC of the invention that are more active than the control conjugate on cell lines expressing CD44 on their surface. EDC3.1 was determined to have 5.5 drugs per antibody on average while EDC3.2 contained 2.7 drugs per antibody on average. The CD44 specific EDC3.1 was most active on non-small-cell lung and pancreatic cancer cells. Both EDC displayed better activity than the control conjugate (containing an average of 7.4 drugs per antibody). Cancer cells tested included colon, non-small-cell lung, pancreatic, breast, head and neck and small-cell lung.

Another example of a targeting moiety useful in making an EDC of the invention is one that specifically recognizes and binds CD98 (4F2hc), its heterodimeric partners or the complexes they form (e.g. LAT1, LAT2, GLUT1). CD98 is a type II transmembrane glycoprotein chain of about 80 kDa composed of 529 amino acid residues, which is known to be highly expressed in various types of cancer cells. CD98 is also found to form complexes various nutrient transporters like LAT1, LAT2 and Glucose transporter 1 (GLUT1) (Ohno et al. Am J Physiol Cell Physiol 300: C1047-C1054, 2011). Six types of amino acid transporter proteins that are considered to bind to CD98 are known. Although CD98 is identified as a lymphocyte activation antigen, it is considered to be involved in a great number of biological functions such as cell growth signaling, integrin activation, cell fusion and the like (Haynes et al., J. Immunol., (1981), 126, 1409-1414, Lindsten et al., Mol. Cell Biol., (1988), 8, 3820-3826, Teixeira et al., Eur. J. Biochem., (1991), 202, 819-826, Diaz Jr. et al., J Biol Regul Homeost Agents, (1998) 12, 25-32).

The LAT1/CD98 heterodimeric complex is involved in a major route for the transport of large neutral essential amino acids through the plasma membrane and is overexpressed in many cancers. LAT1 is an ˜40 kDa protein with amino acid transporter activity (via a disulfide bond) and is expressed on the cell membrane. Cancer cells have various mechanisms to ensure dominance in growth. For example, cancer cells overexpress neutral amino acid transporter to uptake essential amino acids necessary for the growth preferentially over surrounding cells. L-type amino acid transporter 1 (LAT1), an amino acid transporter that is specifically and highly expressed in cancer cells, has been cloned (Kanai et al., J. Biol. Chem. (1998), 273, 23629-23632). LAT1 forms a complex with CD98 and transports neutral amino acids having large side chains, such as leucine, valine, phenylalanine, tyrosine, tryptophan, methionine, histidine and the like in a sodium ion-independent manner. In addition, it is known that LAT1 is poorly or not expressed in most normal tissues except for the brain, placenta, bone marrow and testis, but its expression increases together with CD98 in tissues of human malignant tumors such as colorectal cancer, gastric cancer, breast cancer, pancreatic cancer, renal cancer, laryngeal cancer, esophageal cancer, lung cancer and the like (Yanagida et al., Biochem. Biophys. Acta, (2001), 1514, 291-302). It has been reported that when expression of LAT1 is reduced to suppress amino acid uptake, growth of a tumor is suppressed in a mouse model transplanted with cancer (Japanese Patent Laid-Open No. 2000-157286), and suppression of LAT1 activity is thus considered to be a promising approach for cancer therapy. Human antibodies (or a functional fragment of a human antibody) having specific binding ability to CD98 when expressed on the cell membrane of cancer cells have been shown (U.S. Pat. No. 7,943,745).

In one embodiment, the targeting moiety in the EDC is an antibody or other targeting moiety that specifically binds to CD98. In one embodiment, the targeting moiety in the EDC is an antibody or other targeting moiety that binds to the extracellular domain of LAT1 and inhibits growth of tumor cells which overexpress LAT1. In various embodiments, the targeting moiety of the EDC is an antibody that binds to LAT2. In various embodiments, the targeting moiety of the EDC is an antibody that binds to GLUT1. In various embodiments, the targeting moiety of the EDC is an antibody that may be, for example, a monoclonal antibody, e.g. a murine monoclonal antibody, a chimeric antibody, a human antibody, or a humanized antibody. In one embodiment, the humanized antibody may be, for example, a humanized form of anti-CD98. In one embodiment, the EDC of the invention is constructed from the humanized antibody heavy and light chain variable sequences disclosed in U.S. Pat. No. 7,943,745 and that recognize CD98 on the cell surface. In one embodiment, the antibody is an antibody fragment, e.g. a Fab fragment. In one embodiment, the antibody of the EDC binds specifically to CD98.

Example 4 (see also Example 8) describes an illustrative EDC of the invention that targets CD98. The data in the examples show that antibodies specific to CD98 can be used to produce EDC of the invention that are more active on cell lines expressing CD98 on their surface than control conjugate. EDC4.1 was determined to have 3.4 drugs per antibody on average. The CD98 specific EDC was most active on non-small-cell lung cancer cells. Cancer cells tested included non-small-cell lung, pancreatic, breast, head and neck, small cell lung, and myeloma.

Another example of a targeting moiety useful in making an EDC of the invention is one that specifically recognizes and binds to CD87. CD87 is a widely expressed receptor for urokinase plasminogen activator (uPA) and plays a critical role in regulation of cell-surface plasminogen activation. An expanding body of evidence suggests that CD87 is involved in regulation of diverse physiological and pathological processes, including cellular adhesion, cell motility, angiogenesis, tumor invasion, and tumor metastasis (Yimin and Elghetany, Laboratory Hematology, 2003, 9:67-71). Purified human CD87 is a single-chain, highly glycosylated, extracellular protein with a heterogeneous molecular mass of 50 to 60 kd. The urokinase plasminogen activator system is comprised of the serine protease urokinase (uPA), the urokinase cell surface receptor (uPAR or CD87) and plasminogen activator inhibitor-1 (PA1-1). The urokinase plasminogen activator system is one of the factors responsible for neo-vascularization, invasion and metastasis of many solid tumors (Dano et al., Adv. Cancer Res., 1985, 44:139-266). uPAR plays an essential role in the regulated degradation and remodeling of the extracellular matrix by tumor cells and angiogenic endothelial cells (FIG. 1). uPA-uPAR dependent cascades also result in the activation of promatrix metalloproteinase-9 and the activation and release of growth factors and angiogenic factors including HGF, VEGF and TGF.beta.

uPAR is not usually expressed at detectable levels on quiescent cells and must therefore be upregulated before activities of the system are initiated. uPAR expression is stimulated in vitro by agents such as phorbol esters (Lund et al., J. Biol. Chem. 1991, 266:5177-5181), the transformation of epithelial cells and various growth factors and cytokines such as VEGF, bFGF, HGF, IL-1, TNF.alpha., (in endothelial cells) and GM-CSF (in macrophages) (Mignatti et al., J. Cell Biol. 1991, 113:1193-1201; Mandriota et al., J. Biol. Chem. 270:9709-9716; Yoshida et al., Inflammation 1996, 20:319-326). The uPAR expression has the functional consequence of increasing cell motility, invasion, and adhesion (Mandriota et al., supra). More importantly, uPAR appears to be up-regulated in vivo in most human carcinomas examined to date, specifically, in the tumor cells themselves, in tumor-associated endothelial cells undergoing angiogenesis and in macrophages (Pyke et al., Cancer Res. 1993, 53:1911-15) which may participate in the induction of tumor angiogenesis (Lewis et al., J. Leukoc. Biol. 1995, 57:747-751). uPAR expression in cancer patients is present in advanced disease and has been correlated with a poor prognosis in numerous human carcinomas (Hofmann et al., Cancer 1996, 78:487-92; Heiss et al., Nature Med. 1995, 1:1035-39). Moreover, uPAR is not expressed uniformly throughout a tumor but tends to be associated with the invasive margin and is considered to represent a phenotypic marker of metastasis in human gastric cancer. Accordingly, uPAR is essential in the regulated degradation and remodeling of the extracellular matrix by tumor cells and angiogenic endothelial cell. The important role of uPA-uPAR in tumor growth and its abundant expression within tumor, but not normal tissue, makes this system an attractive diagnostic and therapeutic target.

It has been shown that a polyclonal urokinase receptor antibody can reduce tumor volume and detect the presence of occult tumor metastases in vivo (Rabbani and Gladu, Cancer Res. 62:2390-7 2002). High affinity peptides have been made that target CD87 (Ploug et al., Biochemistry 2001, 40, 12157-12168). Peptides to CD87 have been demonstrated to be effective in inhibiting laminin degradation by cancer cells and limiting metastasis in several animal models (Kobayashi et al., Int J Cancer. 1994; 57:727-733), and in some embodiments are employed as the targeting moiety portion of EDC of the invention. Several fusion proteins of the amino terminal fragment of uPA and the toxin derived from Pseudomonas exotoxin have been described as cytotoxic to several tumor cells at low concentration (Rajagopal and Kreitman, J Biol Chem. 2000; 275:7566-7573). Adenovirus mediated delivery of the uPA-ATF (Li et al., Gene Therapy 5 1105-1113 1998; Human Gene Therapy 16: 1157-1167 2005), stable transfection of uPA-ATF (Zhu et al., DNA Cell Biol 20: 297-305 2001), anti-sense uPAR (Mohan et al., Cancer Res 59: 3369-3373 1999) or combined antisense uPAR/uPA (Gondi et al., Oncogene 22: 5967-5975 2003) resulted in blockade or loss of uPAR activity and inhibition of invasion in vitro and tumor growth and invasion in vivo. In addition, a peptide derived from the non-receptor binding region of urokinase plasminogen activator (uPA) inhibited tumor progression and angiogenesis and induced tumor cell death in vivo (Guo et al., FASEB J. 14:1400-1410 2000). Monoclonal antibodies that specifically binds to urokinase-type plasminogen activator receptor (uPAR) and inhibit binding of urokinase-type plasminogen activator (uPA) to uPAR has been constructed (see US Patent Applications 20120108797 and 20080199476 and 20080152587 and U.S. Pat. No. 8,026,344).

In one embodiment, the targeting moiety in the EDC is an antibody or other targeting moiety such as a peptide or amino terminal fragment of uPA that specifically binds CD87. In one embodiment, the targeting moiety in the EDC is an antibody or other targeting moiety that binds to the extracellular domain of CD87 and exerts an effect on cells which express CD87. In various embodiments, the targeting moiety of the EDC is an antibody that may be, for example, a monoclonal antibody, e.g. a murine monoclonal antibody, a chimeric antibody, a human antibody, or a humanized antibody. In one embodiment, the monoclonal antibody may be constructed of anti-CD87 heavy and light chain variable sequences. In one embodiment, the antibody may be an antibody fragment, e.g. a Fab fragment. In one embodiment, the antibody of the EDC binds specifically to CD87.

Example 5 (see also Example 8) describes an illustrative EDC of the invention that targets CD87. The data in the examples shows that antibodies specific to CD87 can be used to produce EDC of the invention that are more active on cell lines expressing CD87 on their surface than control conjugate. EDC5.1 was determined to have 2.8 drugs per antibody on average. The CD87 specific EDC was most active on melanoma cell line LOX. Cancer cells tested included large cell lung, colon, non-small-cell lung, pancreatic, breast, head and deck, small cell lung, myeloma and myeloma. The only cell line tested that expresses CD87 on the cell surface was the LOX cells, demonstrating that an EDC produced from a CD87 specific targeting moiety can be cancer type specific. However, under the test conditions employed, the cells were not activated to express CD87, and CD87 expression is expected once the CD87 negative lines are pre-activated with reagents as discussed above. Therefore, EDC of the invention targeting the CD87-Na,K-ATPase complex are generally useful in treating cancers that express such complexes under activation conditions.

Another example of a targeting moiety useful in making an EDC of the invention is one that specifically recognizes and binds to cells infected with prions, as occurs in prion disease and prion-associated diseases. Prion diseases are unique, transmissible, neurodegenerative diseases. Prions consists solely of an alternative conformational isoform of the host-encoded normal prion protein, PrP, that replicates without a nucleic acid. Replication is thought to occur by induction of the infectious conformation in the PrP. The different stable conformations, or “conformers”, of PrP have pioneered the concept of protein conformational diseases within the neurodegenerative diseases stating that a misfolded or misprocessed protein is causative in the pathogenesis of disease (Prusiner et al., 2001 N. Engl. J. Med. 344, 1516-1526 and Taylor et al., 2002 Science 296, 1991-5).

Recently it was show that normal prion proteins produced naturally in the brain interact with the amyloid-β peptides. Blocking the interaction halted neurological defects caused by the accumulation of amyloid-β peptide, hallmarks of Alzheimer's disease (Lauren et al., 2009 Nature 457, 1128-1132). Thus, in one embodiment, the EDC of the invention that target CD230 are useful in the treatment of Alzheimer's disease.

While due to the insolubility of many of the misfolded proteins, structural analysis has been difficult, generation of ligands specific for the misfolded proteins has been key to analyze these protein conformations in their cellular environment (Leliveld, S R et al. 2007 J. Neurosci. Res. 85, 2285-97). The notion that soluble alternatively folded conformers or oligomers of proteins rather than insoluble protein deposits are instrumental in the disease processes has focused efforts to develop conformer or oligomer-specfic ligands. Conformation-specific monoclonal antibodies (mABs) have been raised to the pathologically misfolded conformation of PrP, enabling detection of single conformers of proteins within a population of proteins (Korth et al., 1997 Nature 389, 74-77 and Paramithiotis et al., 2003 Nat. Med. 9, 893-9 and Thackray et al., 2003 Biochem. J. 370, 81-90). These reagents have been used in detecting presence of these disease-associated conformers in tissues or body fluids as a method of identifying asymptomatic or early stage individuals at risk to developing prion disease or related disease conditions.

In one embodiment, the targeting moiety in the EDC is an antibody or other targeting moiety that specifically binds to prion related protein (CD230, prion related protein, PrP, major prion protein) encoded in humans by the gene PRNP. PrP was found to be highly expressed in metastatic gastric cancers compared to nonmetastatic ones by immunohistochemical staining. PrPc significantly promoted the adhesive, invasive, and in vivo metastatic abilities of gastric cancer cell lines [The FASEB Journal. 2006; 20:1886-1888]. PrP expression was detected by immunohistochemistry in patients with gastric cancers, and PrP expression in gastric cancers was significantly higher than that in normal gastric tissues [World J Gastroenterol. 2011 Sep. 21; 17(35): 3986-3993]. In one embodiment, the targeting moiety in the EDC is an antibody or other targeting moiety that binds to the extracellular domain of CD230 and exerts an effect on cells which express CD230. In one embodiment, the targeting moiety in the EDC is a conformation-specific monoclonal antibody or other targeting moiety that specifically binds to different stable conformations, or “conformers”, of PrP. In one embodiment, the targeting moiety in the EDC is an anti-abnormal type prion specific monoclonal antibody which binds with abnormal type prion but does not substantially react with normal type prion. In various embodiments, the targeting moiety of the EDC is an antibody that may be, for example, a monoclonal antibody, e.g. a murine monoclonal antibody, a chimeric antibody, a human antibody, or a humanized antibody. In one embodiment, the antibody may be, for example, monoclonal antibody anti-CD230 clone 3F4 (Br J Haematol. 1999 December; 107(4):804-14.). In one embodiment, the antibody is an antibody fragment, e.g. a Fab fragment. In one embodiment, the antibody of the EDC binds specifically to CD230.

Example 6 (see also Example 8) describes an illustrative EDC of the invention that targets CD230. The data in the examples shows that antibodies specific to CD230 can be used to produce EDC of the invention. EDC6.1 was determined to have 7.2 drugs per antibody on average. The CD230 specific EDC was most active on A549 non-small-lung cancer cells. Cancer cells tested included non-small-cell lung, pancreatic, breast, head and neck, and melanoma. All cell lines tested expressed CD230 on their surface as determined by CD230 fluorescent staining, yet EDC6.1 activity was much lower than that for other EDC discussed above. This could have been due to poor binding affinity, the specificity of the antibody used, or that. the complex is mainly formed in brain tissue cells. Targeting moieties that target diseased related conformations of the prion protein can be used as the EDC targeting moiety and the resulting EDC used to treat prion related diseases.

Another example of a targeting moiety useful in making EDC of the invention is one that recognizes and binds to CD56 or specific isoforms of CD56. CD56 (NCAM) is a tumor associated antigen expressed on small cell lung cancer, neuroblastoma, rhabdomyosarkoma, brain tumors, multiple myelomas and acute myeloid leukaemia. NCAM is encoded by a single gene, containing at least 25 exons. Due to alternative splicing of precursor mRNA, a variety of mature mRNA species and thereby protein isoforms of NCAM can be produced. Three major (six total) NCAM isoforms are generated by alternative splicing of exons 15 and 18 determining the mode of attachment of NCAM to the plasma membrane and the size of the intracellular NCAM domains, respectively. In the nervous system a glycosylphosphatidyl inositol (GPI) anchored 120 kDa isoform is expressed on the surface of glial cells, a transmembrane 140 kDa isoform is expressed on both neurons and glial cells, and a transmembrane 180 kDa isoform is found predominantly on the surface of neurons. In aggressive CD56-positive malignant neoplasms, the 140 kDa isoform is the major if not only CD56 isoform expressed (Gattenlohner et al., 2009 AJP V174, No. 4. 1160-1171). The extracellular part of NCAM comprises five Ig-like homology modules (Ig1, Ig2,Ig3, Ig4 and Ig5) and two fibronectin type III modules (F3.1 and F3.2) (Berezin et al., 2000). NCAM carry an unconventional carbohydrate polymer, poly-.alpha.2,8-sialic acid (PSA). PSA is a polymer of negatively charged N-acetylneuraminic acid (sialic acid) residues in an alpha 2,8 linkage. Studies of NCAM knock-out mice (Cremer et al., Nature, 1994, 367, 455-457) indicate that NCAM is the major if not the only carrier of PSA. Many tumors with neural and endocrine characteristics express PSA-NCAM. For example, PSA-NCAM had been detected in neuroblastomas and medulloblastomas (Figarella-Branger et al., Cancer Res., 1990, 50, 6364-6370), small cell carcinoma of the lung (Patel et al., Int. J. Cancer, 1989, 44, 573-578) and rhabdomyosarcomas, and is possibly related to the invasive and metastatic potential of these tumors (Rougon et al., Polysialic Acid, 1993b). Recently, injection of neuraminidase into a nude mouse model for metastasis showed that removal of PSA on the primary tumor delayed metastasis (Daniel et al, Oncogene, 2001, 20, 997-1004). Thus, the molecule PSA-NCAM and the carbohydrate PSA represent suitable targets for the targeting moiety of EDC of the invention to treat cancer and prevent metastasis.

NCAM is not a simple molecular anchor enabling mechanical cell adhesion but rather an important functional receptor mediating intracellular downstream signaling (Crnic et. al., Cancer Res. 64 (2004) 8630-8638). Doherty and Walsh (1999) describe that NCAM, N-cadherin and L1 stimulate axonal growth by activating the fibroblast growth factor receptor (FGFR) in neurons: CD56 induces increased proliferation and decreased apoptosis in acute myeloid leukemias (AMLs) via the nuclear factor (NF)-kB/bc12 pathway (Gattenloehner et al., Blood 2007, 110:2027-2033).

NCAM (or CD56) expression is correlated with morphogenic events suggesting that NCAM is important during development (Edelman, 1990 Cold Spring Harbor Laboratory Press, 1990: 303-318). Thus, NCAM is believed to be important for the development of the nervous system (Daston et al., 1996 Journal of Neuroscience 1996; 16: 5488-5497) and various organs including the kidney (Lackie et al., 1990 Development 1990; 110: 933-947), the liver (Knittel et al., 1996 American Journal of Pathology 1996; 149: 449-462), the bowel (Romanska et al., 1996, Journal of Pediatric Gastroenterology and Nutrition 1996; 22: 351-358), the heart (Gaardsvoll et al., 1993 European Journal of Cell Biology 1993; 61: 100-107), the gonads (Moller et al., Anatomy and Embryology 1991; 184: 541-548), the pancreas (Moller et al., Molecular Endocrinology 1992; 6: 1332-1342), and the muscles (Landmesser et al., Neutrone 1990; 4-655-667). Therefore, therapeutics such as the NCAM-targeting EDC of the invention capable of influencing NCAM function are useful in conditions of impaired development of these organs by inducing appropriate differentiation of target cells. In the brain, the role of NCAM has been supported by knockout mice that have altered development of certain brain regions, including the olfactory system, the hippocampus, the cerebellum and the retina (Cremer et al., Molecular & Cellular Neurosciences 1997; 8: 323-335).

Upregulation of NCAM expression in cultured cells is a direct result of the loss of adherens junctions, and E-cadherin expression can induce upregulated expression of the NCAM gene. As a consequence, NCAM clusters in lipid rafts together with p59Fyn, leading to FAK phosphorylation and focal adhesions assembly, activities required for cell motility and EMT (Lehembre et al., EMBO (2008) 27, 2603-2615). Ablation of NCAM leads to reduced tumorigenicity of tumour cells (Kren et al., (2007) EMBO J 26: 2832-2842).

Therefore, therapeutics such as the EDC of the invention that target NCAM are are useful in the treatment of cancer.

In one embodiment, the targeting moiety in the EDC is an antibody or other targeting moiety that specifically binds CD56, also known as the neural cell adhesion molecule and NCAM. In one embodiment, the targeting moiety in the EDC is an antibody or other targeting moiety that binds to the extracellular domain of CD56 and exerts an effect on cells that express CD56. In one embodiment, the targeting moiety in the EDC is an antibody or other targeting moiety that binds exclusively to the 140 kDa isoform of CD56. In one embodiment, the targeting moiety binds to the extracellular domain of CD56 and exerts an effect on cells that express CD56. In one embodiment, the targeting moiety in the EDC is an antibody or other targeting moiety that binds PSA on NCAM. In various embodiments, the targeting moiety of the EDC is an antibody that may be, for example, a monoclonal antibody, e.g. a murine monoclonal antibody, a chimeric antibody, a human antibody, or a humanized antibody. In one embodiment, the monoclonal antibody is constructed of anti-CD56 heavy and light chain variable sequences. In one embodiment, the antibody is an antibody fragment, e.g. a Fab fragment. In one embodiment, the antibody of the EDC binds specifically to CD56. In one embodiment, the targeting moiety in the EDC is the antibody N901 or the human version of N901 (See e.g., Griffin et al., J. Immunol. 130:2947-2951 (1983) and U.S. Pat. No. 5,639,641).

Example 7 (see also Example 8) describes an illustrative EDC of the invention that targets CD56. The data in the examples shows that antibodies specific to CD56 can be used to produce EDC of the invention that are more active than control conjugate on cell lines expressing CD56 on their surface. EDC7.1 was determined to have 4.6 drugs per antibody on average while EDC7.2 contained 6.9 drugs per antibody on average. The CD56 specific EDC were most active on the only cell line expressing CD56, small-cell lung cancer cell line H69. On those cells, both EDC tested displayed greater activity than the control conjugate containing an average of 7.4 drugs per antibody. Cancer cells tested included large cell lung, colon, non-small-cell lung, pancreatic, breast, head and neck, small-cell lung, melanoma and myeloma. Therefore, EDC of the invention are useful in treating cancers that express CD56 including lymphoblastic and myeloid leukemias (ALLs/AMLs), malignant melanomas, and numerous carcinomas, such as small cell lung cancer.

Additional targets for the targeting moiety can be used to produce EDC of the invention and used to treat various diseases. The transport of lactic acid across the plasma membrane is of fundamental importance for all mammalian cells. Glycolytic cells (e.g. white muscle fibers and all cells under hypoxic conditions), must rapidly export lactic acid; other tissues import lactic acid to fuel respiration (brain, heart, and red muscle) or gluconeogenesis (liver and kidney). Transport is mediated by a family of proton-linked monocarboxylate transporters (MCT(s)) that are also responsible for the transport of other metabolically important monocarboxylates including the ketone bodies. The MCT family has 14 members, 6 of which have been functionally characterized. Of these, only MCT1-MCT4 catalyze proton-coupled transport of lactate. Lactate transport has been found to be crucial for certain solid tumor growth that require lactate to feed into mitochondrial oxidative metabolism in cancer cells (Martinez-Outschoorn et al., 2010 Cell Cycle 9:17, 3515-3533). MCT1 is expressed in most cells, whereas MCT4 is expressed at high levels only in glycolytic tissues (e.g. white muscle) that must export large amounts of lactic acid. MCT2 is absent in most human tissues, whereas MCT3 expression is largely restricted to the retinal pigment epithelium. MCT1, MCT3, and MCT4 require ancillary glycoproteins, either gp70 (Embigin) or, more often CD147 (Basigin), for their expression at the plasma membrane. MCT2 does not associate with CD147 but requires gp70 for its functional expression. MCTs remain associated with basigin or embigin in the plasma membrane, and this interaction is important for their activity.

In one embodiment, the targeting moiety in the EDC is an antibody or other targeting moiety that specifically binds to a monocarboxylate transporter. In one embodiment, the targeting moiety in the EDC is an antibody or other targeting moiety that specifically binds to the epithelial cell adhesion molecule MCT1 encoded in humans by the solute carrier family 16, member 1 (monocarboxylic acid transporter 1) (SLC16A1) gene. In one embodiment, the targeting moiety in the EDC is an antibody or other targeting moiety that binds to the extracellular domain of MCT1 and inhibits growth of tumor cells which overexpress MCT1. In various embodiments, the targeting moiety of the EDC is an antibody that may be, for example, a monoclonal antibody, e.g. a murine monoclonal antibody, a chimeric antibody, a human antibody, or a humanized antibody. In one embodiment, the humanized antibody is, for example, a humanized form of anti-MCT1. In one embodiment, the antibody is an antibody fragment, e.g. a Fab fragment. In one embodiment, the antibody of the EDC binds specifically to MCT1. In one embodiment, the targeting moiety in the EDC is an antibody or other targeting moiety that binds to the extracellular domain of MCT4 and inhibits growth of tumor cells which overexpress MCT4. In various embodiments, the targeting moiety of the EDC is an antibody that is, for example, a monoclonal antibody, e.g. a murine monoclonal antibody, a chimeric antibody, a human antibody, or a humanized antibody. In one embodiment, the humanized antibody is, for example, a humanized form of anti-MCT4. In one embodiment, the antibody is an antibody fragment, e.g. a Fab fragment. In one embodiment, the antibody of the EDC binds specifically to MCT4.

In one embodiment, the targeting moiety in the EDC is an antibody or other targeting moiety that specifically binds to Glutamate receptor, ionotropic, AMPA 2 (GLUR2; GLURB; HBGR2) a protein that in humans is encoded by the GRIA2 gene. In one embodiment, the targeting moiety in the EDC is an antibody or other targeting moiety that binds to the extracellular domain of GLUR2 and exerts an effect on cells which express GLUR2. In various embodiments, the targeting moiety of the EDC is an antibody that may be, for example, a monoclonal antibody, e.g. a murine monoclonal antibody, a chimeric antibody, a human antibody, or a humanized antibody. In one embodiment, the antibody is an antibody fragment, e.g. a “Fab fragment. In one embodiment, the antibody of. the EDC binds specifically to GLUR2.

In one embodiment, the targeting moiety in the EDC is an antibody or other targeting moiety that specifically binds to ASCT2 encoded in humans by the solute carrier family 1 (neutral amino acid transporter), member 5 (SLC1A5) gene (see US Pat. Ap. Pub. Nos. US20100196392 and US20110135570). In one embodiment, the targeting moiety in the EDC is an antibody or other targeting moiety that binds to the extracellular domain of ASCT2 and inhibits growth of tumor cells which over express ASCT2. In various embodiments, the targeting moiety of the EDC is an antibody that may be, for example, a monoclonal antibody, e.g. a murine monoclonal antibody, a chimeric antibody, a human antibody, or a humanized antibody. In one embodiment, the humanized antibody is, for example, a humanized form of anti-ASCT2. In one embodiment, the humanized antibody is constructed from heavy and light chain variable sequences as disclosed in US Pat. App. Pub. No. US20100196392 that recognizes CD98 on the cell surface. In one embodiment, the antibody is an antibody fragment, e.g. a Fab fragment. In one embodiment, the antibody of the EDC binds specifically to ASCT2.

In one embodiment, the targeting moiety in the EDC is an antibody or other targeting moiety that specifically binds to the epithelial cell adhesion molecule EpCAM (CD326) encoded in humans by the EPCAM gene (see US Pat. App. Pub. No. US20070258978). In one embodiment, the targeting moiety in the EDC is an antibody or other targeting moiety that binds to the extracellular domain of EpCAM and inhibits growth of tumor cells which overexpress EpCAM. In various embodiments, the targeting moiety of the EDC is an antibody that may be, for example, a monoclonal antibody, e.g. a murine monoclonal antibody, a chimeric antibody, a human antibody, or a humanized antibody. In one embodiment, the humanized antibody is, for example, a humanized form of anti-EpCAM. In one embodiment, the humanized antibody is MAb17-1A, a chimeric mouse/human monoclonal antibody to the cell-surface glycoprotein EpCAM. In one embodiment, the humanized antibody is MAb17-1A Adecatumumab (MT201), a recombinant human monoclonal antibody that binds the cell-surface glycoprotein EpCAM (Current Opinion in Molecular Therapeutics 2007 9:190-196). In one embodiment, the antibody is an antibody fragment, e.g. a Fab fragment. In one embodiment, the antibody of the EDC binds specifically to EpCAM.

Various integrins play an important role in tumor angiogenesis. Integrins are transmembrane receptors for extracellular matrix (ECM) and basement membrane proteins that are composed of 2 noncovalently associated subunits, alpha and beta. The integrins alpha v beta 3 and alpha v beta 5 bind to ECM molecules. Pharmacologic agents targeted to integrins have been reported to block tumor and retinal angiogenesis (Maubant et al., Blood, 2006; 108, (9) 3035-3045). Therefore targeting moieties that recognize integrins are useful in EDC of the invention.

In one embodiment, the targeting moiety in the EDC is an antibody or other targeting moiety that specifically binds to beta 3 integrin. In one embodiment, the targeting moiety in the EDC is an antibody or other targeting moiety that specifically binds to alpha 5 integrin. In one embodiment, the targeting moiety in the EDC is an antibody or other targeting moiety that specifically binds to alpha 1 integrin. In various embodiments, the targeting moiety in the EDC is an anti-integrin antibody as described in Byron et al., J Cell Science 2009; 122, 4009-4011 .

In one embodiment, the targeting moiety in the EDC is an antibody or other targeting moiety that specifically binds to the activated leukocyte cell adhesion molecule (CD166), encoded in humans by the ALCAM gene. In one embodiment, the targeting moiety in the EDC is an antibody or other targeting moiety that binds to the extracellular domain of CD 166 and inhibits growth of tumor cells which overexpress CD166. In various embodiments, the targeting moiety of the EDC is an antibody that may be, for example, a monoclonal antibody, e.g. a murine monoclonal antibody, a chimeric antibody, a human antibody, or a humanized antibody. In one embodiment, the antibody is an antibody fragment, e.g. a Fab fragment. In one embodiment, the antibody of the EDC binds specifically to CD166.

In one embodiment, the targeting moiety in the EDC is an antibody or other targeting moiety that specifically binds to the transferrin receptor (CD71, p90, TFR1) encoded in humans by the TFRC gene (see U.S. Pat. No. 7,736,647 and US Pat. App. Pub. No. US20060039907). In one embodiment, the targeting moiety in the EDC is an antibody or other targeting moiety that binds to the extracellular domain of CD71 and inhibits growth of tumor cells which overexpress CD71. In various embodiments, the targeting moiety of the EDC is an antibody that may be, for example, a monoclonal antibody, e.g. a murine monoclonal antibody, a chimeric antibody, a human antibody, or a humanized antibody. In one embodiment, the humanized antibody is constructed from heavy and light chain variable sequences (see U.S. Pat. No. 7,736,647) that recognize CD71 on the cell surface. In one embodiment, the antibody is an antibody fragment, e.g. a Fab fragment. In one embodiment, the antibody of the EDC binds specifically to CD71.

In one embodiment, the targeting moiety in the EDC is an antibody or other targeting moiety that specifically binds to the sel-1 suppressor of lin-12-like (C. elegans) (SEL1L1, IBD2) encoded in humans by the SEL1L gene. In one embodiment, the targeting moiety in the EDC is an antibody or other targeting moiety that binds to the extracellular domain of SEL1L1 and inhibits growth of tumor cells which over express SEL1L1. In various embodiments, the targeting moiety of the EDC is an antibody that may be, for example, a monoclonal antibody, e.g. a murine monoclonal antibody, a chimeric antibody, a human antibody, or a humanized antibody. In one embodiment, the antibody is an antibody fragment, e.g. a Fab fragment. In one embodiment, the antibody of the EDC binds specifically to SEL1L.

In one embodiment, the targeting moiety in the EDC is an antibody or other targeting moiety that specifically binds to retinoschisin 1 (retinoschisin, RS, XLRS2) encoded in humans by the RS1 gene (see US Pat. App. Pub. No. US20100047779). In one embodiment, the targeting moiety in the EDC is an antibody or other targeting moiety that binds to the extracellular domain of retinoschisin and exerts an effect on cells which express retinoschisin. In various embodiments, the targeting moiety of the EDC is an antibody that may be, for example, a monoclonal antibody, e.g. a murine monoclonal antibody, a chimeric antibody, a human antibody, or a humanized antibody. In one embodiment, the antibody is an antibody fragment, e.g. a Fab fragment. In one embodiment, the antibody of the EDC binds specifically to retinoschisin.

In one embodiment, the targeting moiety in the EDC is an antibody or other targeting moiety that specifically binds to toll-like receptor 4 (CD284, TLR4, TOLL) encoded in humans by the TLR4 gene (see US Pat. App. Pub. Nos. US20100183619 and US20100266619). In one embodiment, the targeting moiety in the EDC is an antibody or other targeting moiety that binds to the extracellular domain of TLR4 and exerts an effect on cells which express TLR4. In various embodiments, the targeting moiety of the EDC is an antibody that may be, for example, a monoclonal antibody, e.g. a murine monoclonal antibody, a chimeric antibody, a human antibody, or a humanized antibody. In one embodiment, the humanized antibody is constructed from heavy and light chain variable sequences (see US20100183619) that recognize TLR4 on the cell surface. In one embodiment, the antibody is an antibody fragment, e.g. a Fab fragment. In one embodiment, the antibody of the EDC binds specifically to TLR4.

In one embodiment, the targeting moiety in the EDC is an antibody or other targeting moiety that specifically binds to inositol 1,4,5-triphosphate receptor, type 1 (IP3R1, INSP3R1) encoded in humans by the ITPR1 gene. In one embodiment, the targeting moiety in the EDC is an antibody or other targeting moiety that binds to the extracellular domain of IP3R1 and exerts an effect on cells which express IP3R1. In various embodiments, the targeting moiety of the EDC is an antibody that may be, for example, a monoclonal antibody, e.g. a murine monoclonal antibody, a chimeric antibody, a human antibody, or a humanized antibody. In one embodiment, the antibody is an antibody fragment, e.g. a Fab fragment. In one embodiment, the antibody of the EDC binds specifically to IP3R1.

In one embodiment, the targeting moiety in the EDC is an antibody or other targeting moiety that specifically binds to a member of the fibroblast growth factor receptor family (see US Pat. App. Nos. US20110135657 and US20100247531). In one embodiment, the targeting moiety in the EDC is an antibody or other targeting moiety that binds to the extracellular domain of FGFR1 (CD331) and exerts an effect on cells which express FGFR1. In another embodiment, the targeting moiety in the EDC is an antibody or other targeting moiety that binds to the extracellular domain of FGFR2 (CD332) and exerts an effect on cells which express FGFR2. In another embodiment, the targeting moiety in the EDC is an antibody or other targeting moiety that binds to the extracellular domain of FGFR3 (CD333) and exerts an effect on cells which express FGFR3. In another embodiment, the targeting moiety in the EDC is an antibody or other targeting moiety that binds to the extracellular domain of FGFR4 (CD334) and exerts an effect on cells which express FGFR4. In various embodiments, the targeting moiety of the EDC is an antibody that may be, for example, a monoclonal antibody, e.g. a murine monoclonal antibody, a chimeric antibody, a human antibody, or a humanized antibody. In one embodiment, the antibody is an antibody fragment, e.g. a Fab fragment. In one embodiment, the antibody of the EDC binds specifically to FGFR1. In one embodiment, the antibody of the EDC binds specifically to FGFR2. In one embodiment, the antibody of the EDC binds specifically to FGFR3. In one embodiment, the antibody of the EDC binds specifically to FGFR4. In one embodiment, the humanized antibody may be IMC-A1 (a fully human monoclonal antibody that recognizes FGFR1c on the cell surface(see Am J Physiol Endocrinol Metab 292: E964-E976, 2007). In one embodiment, the humanized antibody is R3Mab (a fully human monoclonal antibody that recognizes FGFR3 on the cell surface, see J. Clin. Invest. 119 (5) May 2009). In one embodiment the targeting moiety in the EDC is an antibody or other targeting moiety that specifically binds to a member of the fibroblast growth factor receptor family. In one embodiment the targeting moiety is a recombinant form of fibroblast growth factor such as FGF1, FGF2, FGF3, or FGF7.

In one embodiment, the targeting moiety in the EDC is an antibody or other targeting moiety that specifically binds to Klotho beta (beta-Klotho, BKL) encoded in humans by the gene KLB (see US Pat. App. Pub. No. US20110135657). In one embodiment, the targeting moiety in the EDC is an antibody or other targeting moiety that binds to the extracellular domain of Klotho beta and exerts an effect on cells which express Klotho beta. In various embodiments, the targeting moiety of the EDC is an antibody that may be, for example, a monoclonal antibody, e.g. a murine monoclonal antibody, a chimeric antibody, a human antibody, or a humanized antibody. In one embodiment, the humanized antibody is constructed from heavy and light chain variable sequences (see US20110135657) that recognize Klotho beta on the cell surface. In one embodiment, the antibody is an antibody fragment, e.g. a Fab fragment. In one embodiment, the antibody of the EDC binds specifically to Klotho beta.

In one embodiment, the targeting moiety in the EDC is an antibody or other targeting moiety that specifically binds proteins of the T cell receptor (TCR) complex. In one embodiment, the targeting moiety in the EDC is an antibody or other targeting moiety that binds to the extracellular domain of CD3 and exerts an effect on cells which express CD3. In one embodiment the antibody of the EDC binds specifically to the TCRα Vα7.2 segment (see Martin et al. (2009) Stepwise Development of MAIT Cells in Mouse and Human. PLoS Biol 7(3): e1000054. doi:10.1371/journal.pbio.1000054). In one embodiment the antibody of the EDC binds specifically to T cell receptor (TCR) Vbeta5.2/5.3 (Eur J Neurol. 2002 March; 9(2):153-64.). In various embodiments, the targeting moiety of the EDC is an antibody that may be, for example, a monoclonal antibody, e.g. a murine monoclonal antibody, a chimeric antibody, a human antibody, or a humanized antibody. In one embodiment, the mouse monoclonal antibody is 3C10 anti-TCRa Va7.2 that recognizes T cell receptor (TCR) Va7.2 on the cell surface. In one embodiment, the humanized antibody is ATM-027 that recognizes T cell receptor (TCR) Vbeta5.2/5.3 on the cell surface (Ann Neurol. 2002 April; 51(4):467-74). In one embodiment, the humanized antibody is Visilizumab that binds to the CD3 receptor on certain activated T cells. In one embodiment, the antibody is the mouse mAb Muromonab-CD3 that binds to the CD3 receptor on T cells. In one embodiment, the antibody is the chimeric/humanized mAb Otelixizumab, also known as TXR4, which targets CD3. In one embodiment, the antibody is the humanized mAb Teplizumab, also known as MGA031, which targets CD3. In one embodiment, the antibody is an antibody fragment, e.g. a Fab fragment. In one embodiment, the antibody of the EDC binds specifically to a protein of the TCR complex.

In one embodiment, the targeting moiety in the EDC is an antibody or other targeting moiety that specifically binds the insulin-like growth factor 1 receptor (IGFR, CD221, IGF1R) a protein that in humans is encoded by the IGF1R gene (Current Opinion in Drug Discovery and Development 2008 11:178-185, Combinatorial Chemistry & High Throughput Screening, Volume 11, Number 1, January 2008, pp. 62-69(8)). In one embodiment, the targeting moiety in the EDC is an antibody or other targeting moiety that binds to the extracellular domain of IGFR and exerts an effect on cells which express IGFR. In various embodiments, the targeting moiety of the EDC is an antibody that may be, for example, a monoclonal antibody, e.g. a murine monoclonal antibody, a chimeric antibody, a human antibody, or a humanized antibody. In one embodiment, the monoclonal antibody is AMG 479, a fully human monoclonal antibody that recognizes IGF1R on the cell surface. In one embodiment, the monoclonal antibody is Figitumumab (CP-751,871), a fully human monoclonal antibody that recognizes IGF1R on the cell surface (Clinical Lung Cancer, Volume 10, Number 4/July 2009). In one embodiment, the monoclonal antibody is SCH 717454 (Robatumumab), a fully human monoclonal antibody that recognizes IGF1R on the cell surface (Mol Cancer Ther 2010; 9:410-418.) In one embodiment, the monoclonal antibody is IMC-A12 (Cixutumumab), a fully human monoclonal antibody that recognizes IGF1R on the cell surface (Clin Cancer Res 2007; 13:5549s-5555s.) In another embodiment, the targeting moiety in the EDC is an antibody or other targeting moiety that specifically binds to the insulin like growth factor 1 receptor. In one embodiment, the antibody is an antibody fragment, e.g. a Fab fragment. In one embodiment, the antibody of the EDC binds specifically to IGF1R

In one embodiment, the targeting moiety in the EDC is an antibody or other targeting moiety that specifically binds tumor necrosis factor alpha (TNF-alpha), a protein that in humans is encoded by the TNF gene (Immunol. Cell Biol. 74 (5): 465-72). In one embodiment, the targeting moiety in the EDC is an antibody or other targeting moiety that binds to the extracellular domain of the transmembrane-form of TNF-alpha and exerts an effect on cells which express TNF-alpha. In various embodiments, the targeting moiety of the EDC is an antibody that may be, for example, a monoclonal antibody, e.g. a murine monoclonal antibody, a chimeric antibody, a human antibody, or a humanized antibody. In one embodiment, the monoclonal antibody is Infliximab (INN; trade name Remicade), which is a mouse/human chimeric monoclonal antibody specific for tumour necrosis factor alpha (Gastroenterology Volume 124, Issue 7, Pages 1774-1785, July 2003). In one embodiment, the monoclonal antibody is Adalimumab (HUMIRA, D2E7), a fully human monoclonal antibody that recognizes tumor necrosis factor alpha (see U.S. Pat. No. 6,090,382). In one embodiment, the antibody is an antibody fragment, e.g. a Fab fragment. In one embodiment, the antibody of the EDC binds specifically to TNF-alpha

In one embodiment, the targeting moiety in the EDC is an antibody or other targeting moiety that specifically binds membrane-associated phospholipase A2 (PLA2) (Am J Physiol. 1998 February; 274(2 Pt 1):C447-54. PMID:9486135). In one embodiment, the targeting moiety in the EDC is an antibody or other targeting moiety that binds to the extracellular domain of membrane associated PLA2 and exerts an effect on cells which express PLA2. In various embodiments, the targeting moiety of the EDC is an antibody that may be, for example, a monoclonal antibody, e.g. a murine monoclonal antibody, a chimeric antibody, a human antibody, or a humanized antibody. In one embodiment, the humanized antibody is constructed from heavy and light chain variable sequences (see US Pat. App. Pub. No. US20050058649) that recognize PLA2. In one embodiment, the antibody is an antibody fragment, e.g. a Fab fragment. In one embodiment, the antibody of the EDC binds specifically to PLA2.

In one embodiment, the targeting moiety in the EDC is an antibody or other targeting moiety that specifically binds phosphatidylethanolamine N-methyltransferase (PE-NMT, PEMT, PEMPT) a protein that in humans that is encoded by the PEMT gene (see Biochim Biophys Acta. 1999 Jan. 4; 1436(3):405-12 and Morrill et al. BMC Structural Biology 2010, 10:12). In one embodiment, the targeting moiety in the EDC is an antibody or other targeting moiety that binds to the extracellular domain of PEMT and exerts an effect on cells which express PEMT. In various embodiments, the targeting moiety of the EDC is an antibody that may be, for example, a monoclonal antibody, e.g. a murine monoclonal antibody, a chimeric antibody, a human antibody, or a humanized antibody. In one embodiment, the antibody is an antibody fragment, e.g. a Fab fragment. In one embodiment, the antibody of the EDC binds specifically to PEMT.

In one embodiment, the targeting moiety in the EDC is an antibody or other targeting moiety that specifically binds angiotensin II receptor, type 1 (AGTR1A, AT2R1) a protein that in humans that is encoded by the AGTR1 gene (see U.S. Pat. No. 6,805,864 and J Physiol 586.22 (2008) pp 5337-5348, Am J Physiol Renal Physiol 294: F990-F1000, 2008). In one embodiment, the targeting moiety in the EDC is an antibody or other targeting moiety that binds to the extracellular domain of AGTR1A and exerts an effect on cells which express AGTR1A. In various embodiments, the targeting moiety of the EDC is an antibody that may be, for example, a monoclonal antibody, e.g. a murine monoclonal antibody, a chimeric antibody, a human antibody, or a humanized antibody. In one embodiment, the monoclonal antibody is a monoclonal antibody described in U.S. Pat. No. 6,805,864 (see claim 2). In one embodiment, the antibody is an antibody fragment, e.g. a Fab fragment. In one embodiment, the antibody of the EDC binds specifically to AGTR1A.

In one embodiment, the targeting moiety in the EDC is an antibody or other targeting moiety that specifically binds the solute carrier family 6 (neurotransmitter transporter, serotonin), member 4 (SLC6A4; HTT; 5-HTT; 5HTT; OCD1; SERT; hSERT), a protein that in humans that is encoded by the SLC6A4 gene (J. Phar. Exp. Ther. 285:835-843, 1998). In one embodiment, the targeting moiety in the EDC is an antibody or other targeting moiety that binds to the extracellular domain of SERT and exerts an effect on cells which express SERT. In various embodiments, the targeting moiety of the EDC is an antibody that may be, for example, a monoclonal antibody, e.g. a murine monoclonal antibody, a chimeric antibody, a human antibody, or a humanized antibody. In one embodiment, the antibody is an antibody fragment, e.g. a Fab fragment. In one embodiment, the antibody of the EDC binds specifically to SERT.

RANK (Receptor Activator of Nuclear Factor Kappa B) also known as TRANCE Receptor, is expressed on the surface of stromal cells, osteoblasts, and T cells. RANK expression has also been found in cancer cell lines from human origin such as osteosarcoma, breast and prostate carcinomas (Santini et al., 2011 PLoS ONE 6(4): e19234). RANKL (CD254) is the ligand that binds to RANK. RANKL is also a surface bound molecule. RANKL is critical for adequate bone metabolism and overproduction of RANKL is implicated in a variety of degenerative bone diseases, such as rheumatoid arthritis and psoriatic arthritis. EDC of the invention that target RANK or RANKL are useful in treating degenerative bone diseases, such as rheumatoid arthritis and psoriatic arthritis.

Therefore, in one embodiment, the targeting moiety in the EDC is an antibody or other targeting moiety that specifically binds the tumor necrosis factor (ligand) superfarriily, member 11 (TNFSF11; ODF; CD254; OPGL; RANKL; TRANCE; hRANKL2; sOdf) a protein that in humans is encoded by the TNFSF11 gene (see US Pat. App. Pub. No. US20070134245 and US20020086826; and U.S. Pat. No. 7,411,050). In one embodiment, the targeting moiety in the EDC is an antibody, or other targeting moiety that binds to the extracellular domain of RANKL and exerts an effect on cells which express RANKL. In various embodiments, the targeting moiety of the EDC is an antibody that may be, for example, a monoclonal antibody, e.g. a murine monoclonal antibody, a chimeric antibody, a human antibody, or a humanized antibody. In one embodiment, the monoclonal antibody is constructed of anti-RANKL heavy and light chain variable sequences as described in US20070134245. In one embodiment, the antibody is an antibody fragment, e.g. a Fab fragment. In one embodiment, the antibody of the EDC binds specifically to RANKL.

In another embodiment, the targeting moiety in the EDC is an antibody or other targeting moiety that binds to the extracellular domain of RANK and exerts an effect on cells which express RANK. In one embodiment, the targeting moiety in the EDC is an antibody or other targeting moiety that binds to the extracellular domain of RANKL and exerts an effect on cells which express RANK.

Antibody targeting moieties in the EDC of the invention typically retain the antigen binding capability of their native, unconjugated counterparts. Thus, antibodies useful in the EDC of the invention are capable of binding specifically to antigens while covalently linked to an agent that acts on the Na,K-ATPase (e.g. scillarenin) through a stable (and, in some embodiments, non-cleavable) linker. Such antigens include proteins or targets that are associated with and in close proximity to the Na,K-ATPase in cells or tissues being targeted for therapeutic intervention (or diagnosis).

Various methods have been employed to produce monoclonal antibodies (MAbs), and these methods are applicable to the production of antibodies for use in the EDC of the invention and so are briefly reviewed below. Hybridoma technology, which refers to a cloned cell line that produces a single type of antibody, uses the cells of various species, including mice (murine), hamsters, rats, and humans. Other methods to prepare MAbs, including chimeric and humanized antibodies, employ genetic engineering, e.g. recombinant DNA techniques.

Polyclonal antibodies may be raised in animals by multiple subcutaneous (sc) or intraperitoneal (ip) injections of the relevant antigen and an adjuvant. Monoclonal antibodies are obtained from a population of substantially homogeneous antibodies, e.g., the individual antibodies comprising the population are identical except for possible naturally occurring mutations that may be present in minor amounts.

Human myeloma and mouse-human heteromyeloma cell lines also have been described for the production of human monoclonal antibodies (Kozbor, (1984) J. Immunol., 133:3001, and Brodeur et al., Monoclonal Antibody Production Techniques and Applications, pp. 51-63 (Marcel Dekker, Inc., New York, 1987)). Culture medium in which hybridoma cells are growing is assayed for production of monoclonal antibodies directed against the antigen. Binding specificity of monoclonal antibodies produced by hybridoma cells may be determined by immunoprecipitation or by an in vitro binding assay, such as radioimmunoassay (RIA) or enzyme-linked immunoabsorbent assay (ELISA). The binding affinity of the monoclonal antibody can, for example, be determined by the Scatchard analysis of Munson et al (1980) Analyt. Biochem. 107:220.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells (see U.S. Pat. App. Pub. Nos. US20050048572 and US20040229310). Review articles on recombinant expression in bacteria of DNA encoding the antibody include Skerra et al (1993) Curr. Opinion in Immunol. 5:256-262 and Pluckthun (1992) Immunol. Revs. 130:151-188.

In a further embodiment, monoclonal antibodies or antibody fragments can be isolated from antibody phage libraries generated using the techniques described in McCafferty et al (1990) Nature 348:552-554; Clackson et al (1991) Nature 352:624-628; and Marks et al (1991) J. Mol. Biol., 222:581-597 describe the isolation of murine and human antibodies, respectively, using phage libraries. Subsequent publications describe the production of high affinity (nM range) human antibodies by chain shuffling (Marks et al (1992) Bio/Technology 10:779-783), as well as combinatorial infection and in vivo recombination as a strategy for constructing very large phage libraries (Waterhouse et al (1993) Nuc. Acids. Res. 21:2265-2266). Thus, these techniques are viable alternatives to traditional monoclonal antibody hybridoma techniques for isolation of monoclonal antibodies.

The DNA also may be modified, for example, by substituting the coding sequence for human heavy chain and light chain constant domains in place of the homologous murine sequences (U.S. Pat. No. 4,816,567); and Morrison et al (1984) Proc. Natl. Acad. Sci. USA 81:6851), or by covalently joining to the immunoglobulin coding sequence all or part of the coding sequence for a non-immunoglobulin polypeptide.

Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

As an alternative to humanization, human antibodies can be generated. For example, it is now possible to produce transgenic animals (e.g., mice) that are capable, upon immunization, of producing a full repertoire of human antibodies in the absence of endogenous immunoglobulin production (Jakobovits et al., (1993) Proc. Natl. Acad. Sci. USA, 90:2551; Jakobovits et al., (1993) Nature 362:255-258; Bruggermann et al., (1993) Year in Immuno. 7:33; and U.S. Pat. Nos. 5,591,669; 5,589,369; and 5,545,807).

Alternatively, phage display technology (McCafferty et al., (1990) Nature 348:552-553) can be used to produce human antibodies and antibody fragments in vitro, from immunoglobulin variable (V) domain gene repertoires from unimmunized donors (Johnson et al., (1993) Curr. Opin. Structural Biol. 3:564-571). A repertoire of V genes from unimmunized human donors can be constructed and antibodies to a diverse array of antigens (including self-antigens) can be isolated essentially (Marks et al., (1991) J. Mol. Biol. 222:581-597; Griffith et al., (1993) EMBO J. 12:725-734; U.S. Pat. Nos. 5,565,332 and 5,573,905). Human antibodies may also be generated by in vitro activated B cells (U.S. Pat. Nos. 5,567,610 and 5,229,275). Human anti-ErbB2 antibodies have been described (U.S. Pat. No. 5,772,997 and PCT Pub. No. WO 97/00271.

Various techniques have been developed for the production of antibody fragments. Traditionally, these fragments were derived via proteolytic digestion of intact antibodies (see Morimoto et al., (1992) J. Biochem. Biophys. Methods 24:107-117; and Brennan et al., (1985) Science 229:81). Antibody fragments can also be produced directly by recombinant host cells and the antibody phage libraries discussed above. Fab′-SH fragments can be directly recovered from E. coli and chemically coupled to form F(ab′)₂ fragments (Carter et at (1992) Bio/Technology 10:163-167). According to another approach, F(ab′)2 fragments can be isolated directly from recombinant host cell culture. Other techniques for the production of antibody fragments will be apparent to the skilled practitioner. In other embodiments, the antibody of choice is a single chain Fv fragment (v (sFv) dimers (Gruber et al., (1994) J. Immunol. 152:5368). Techniques for -generating bispecific antibodies from antibody fragments have also been described, such as using chemical linkage wherein intact antibodies are proteolytically cleaved to generate F(ab′)2 fragments (Brennan et al., (1985) Science 229:81). Fab′-SH fragments can be recovered from E. coli and chemically coupled to form bispecific antibodies (Shalaby et al., (1992) J. Exp. Med. 175:217-225. The “diabody” technology provides an alternative method for making bispecific antibody fragments (Hollinger et al., (1993) Proc. Natl. Acad. Sci. USA 90:6444-6448).

Antibodies with more than two valencies can be employed in various embodiments of the EDC of the invention. Multivalent, “Octopus” antibodies with three or more antigen binding sites and two or more variable domains can be readily produced by recombinant expression of nucleic acid encoding the polypeptide chains of the antibody (US Pat. App. Pub. No. US20020004586 and PCT Pub. No. WO 01/77342). For example, trispecific antibodies can be prepared (Tutt et al., (1991) J. Immunol. 147:60).

Amino acid sequence modification(s) of antibodies are contemplated by the invention. For example, mutants and various isoforms of antibodies which bind to tumor-associated or other antigens are contemplated to improve the binding affinity and/or other biological properties of the antibody and/or allow for site specific conjugation of the linker and/or therapeutic agent to the antibody. Amino acid sequence variants of an antibody are prepared by introducing appropriate nucleotide changes into the nucleic acid encoding the antibody, or by peptide synthesis. Such modifications include, for example, deletions from, and/or insertions into and/or substitutions of, residues within the amino acid sequences of the antibody. Any combination of deletion, insertion, and substitution is made to arrive at the final construct, provided that the final construct possesses the desired characteristics. The amino acid changes also may alter post-translational processes of the antibody, such as changing the number or position of glycosylation sites.

A useful method for identification of certain residues or regions of the antibody that are preferred locations for mutagenesis is “alanine scanning mutagenesis” (Cunningham and Wells (1989) Science 244:1081-1085) where an amino acid residue, or group of target residues, are identified (e.g., charged residues such as arg, asp, his, lys, and glu) and replaced by a neutral or negatively charged amino acid, such as alanine or polyalanine, to optimize the interaction of the amino acids with antigen. Amino acid sequence insertions include amino-and/or carboxyl-terminal fusions ranging in length from one residue to polypeptides containing a hundred or more residues, as well as intrasequence insertions of single or multiple amino acid residues.

The amino acid sequence of an antibody is usually altered by altering the underlying nucleic acid sequence. Nucleic acid molecules encoding amino acid sequence variants of the antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody. The sites of greatest interest for substitutional mutagenesis include the hypervariable regions, but FR alterations are also contemplated.

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties: (1) hydrophobic: norleucine, met, ala, val, leu, ile; (2) neutral hydrophilic: cys, ser, thr; (3) acidic: asp, glu; (4) basic: asn, gln, his, lys, arg; (5) residues that influence chain orientation: gly, pro; and (6) aromatic: trp, tyr, phe. Non-conservative substitutions will entail exchanging a member of one of these classes for another class.

Any cysteine residue not involved in maintaining the proper conformation of the antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

To increase the serum half life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule (see US Pat. App. Pub. No. US20030190311, and U.S. Pat. Nos. 6,821,505; 6,165,745; 5,834,597; 5,648,260; and 5,624,821). PEGylation can also be used to increase the half life of an EDC of the invention.

Glycosylation variants of antibodies are variants in which the glycosylation pattern of an antibody is altered. By altering is meant deleting one or more carbohydrate moieties found in the antibody, adding one or more carbohydrate moieties to the antibody, changing the composition of glycosylation (glycosylation pattern), or the extent of glycosylation. Antibodies may be glycosylated at conserved positions (N-linked or O-linked) in their constant regions (Hse et al., (1997) J. Biol. Chem. 272:9062-9070; Jefferis and Lund, (1997) Chem. Immunol. 65:111-128; Wright and Morrison, (1997) TibTECH 15:26-32). The oligosaccharide side chains of the immunoglobulins affect the protein's function (Boyd et al., (1996) Mol. Immunol. 32:1311-1318; Wittwe and Howard, (1990) Biochem. 29:4175-4180), and the intramolecular interaction between portions of the glycoprotein which can affect the conformation and presented three-dimensional surface of the glycoprotein (Hefferis and Lund, supra; Wyss and Wagner (1996) Current Opin. Biotech. 7:409-416). Oligosaccharides may also serve to target a given glycoprotein to certain molecules based upon specific recognition structures (Malhotra et al., (1995) Nature Med. 1:237-243; Umana et al., (1999) Nature Biotech. 17:176-180). Removal of the oligosaccharides may optimize antigen binding and other properties of the antibody (Boyd et al., (1996) Mol. Immunol. 32:1311-1318).

Factors which affect glycosylation during recombinant production of antibodies include growth mode, media formulation, culture density, oxygenation, pH, purification schemes and the like (U.S. Pat. No. 5,047,335; 5,278,299; and 5,510,261). Glycosylation, or certain types of glycosylation, can be enzymatically removed from the glycoprotein, for example using endoglycosidase H (Endo H). In addition, the recombinant host cell can be genetically engineered, e.g. make defective in processing certain types of polysaccharides. These and similar techniques are well known in the art.

The glycosylation structure of antibodies can be readily analyzed by conventional techniques of carbohydrate analysis, including lectin chromatography, NMR, Mass spectrometry, HPLC, GPC, monosaccharide compositional analysis, sequential enzymatic digestion, and HPAEC-PAD, which uses high pH anion exchange chromatography to separate oligosaccharides based on charge. Methods for releasing oligosaccharides for analytical purposes are also known, and include, without limitation, enzymatic treatment (commonly performed using peptide-N-glycosidase F/endo-.beta.-galactosidase), elimination using harsh alkaline environment to release mainly O-linked structures, and chemical methods using anhydrous hydrazine to release both N- and O-linked oligosaccharides.

Some of these targets of antibodies or other targeting moieties of the EDC of the invention have multiple subunits, isoforms and/or glycosylation patterns which determine their location in or on cells. Their presentation can depend on cell type, location on the cell, location of the cell, and/or physiological and pathological conditions. For example, aberrant glycosylation is a hallmark of cancer and includes alterations in the carbohydrate content of glycoproteins, glycolipids, and glycosaminoglycans (see Anticancer Agents Med Chem 2008; 8(1):2-21, incorporated herein by reference). Specifically, there is an abundance of evidence that beta-1,6-GlcNAc-branching of N-glycans contributes directly to cancer progression (see Biochim Biophys Acta 1999; 1473(1):21-34, incorporated herein by reference). As another example, the type of beta subunit (1 vs. 2) found in the Na/K-ATPase complex and its glycosylation pattern differ from cell type to cell type (see Proteomics 2008; 8(16):3236-56, and Am J Physiol 1997; 272(1 Pt 1):L85-94, incorporated herein by reference). The glycosylation patterns of the Na/K-ATPase membrane bound ion pump are believed to have evolved to serve cell specific regulatory requirements, and aberrant glycosylation patterns have been identified in a number of cancer cells. In addition, gamma subunit isoform 5 of the Na/K-ATPase membrane bound ion pump complex is also a glycosylated membrane protein (also called dysadherin or FXYD5) that has been shown to promote experimental cancer metastasis and is an independent prognostic indicator of metastasis and survival for many different types of human cancer [see Nam et. al. Cancer Lett. 255(2) 161-9 (2007)]. In addition, many extracellular targets are slightly different or over expressed in diseased tissues or in the circulatory structure surrounding diseased tissue. Accordingly, any of the target antigens resulting from aberrant or unusual glycosylation or altered expression of antigens can be targeted by the targeting moiety of the EDC of the invention.

A somatic mutation in the chaperone Cosmc can lead to the creation of new glycopeptide epitopes to which cancer specific antibodies for use in the EDC of the invention can be raised (see Schietinger et al., Science 314(5797) 304-8 (2006), incorporated herein by reference). Antibodies have been generated that recognize these differences in glycosylation. Two papers describe how to produce specific antibodies to aberrantly glycosylated cell surface glycans (see Cancer Immunol Immunother 2006; 55(11):1337-47, and Cancer Res 2009; 69(5):2018-25). However, generating high affinity antibodies to sugars alone can be difficult. To overcome the problem of immune tolerance to tumor-associated carbohydrate antigens, non-naturally occurring antigenic sugars can be fed to cells, creating new glycosylation motifs to which high affinity antibodies can be generated (see Bioorg. Med. Chem. 15 (2007) 7561-7567). Antibodies generated to these non-naturally occurring sugars in conjunction with the proteins they decorate lead to very specific disease targeting antibodies and are useful in various embodiments of the EDC of the invention.

Antibodies have also been generated to targets that are over-expressed in diseased tissue, and such antibodies are useful in the EDC of the invention when directed to proteins that associate with the Na,K-ATPase. For cancer, these antigens are typically named tumor-associated antigens and represent a group of normal non-mutant molecules. For example, antibodies have been generated to targets that are over-expressed on cancer cells that metastasize, and such antibodies are useful in the EDC of the invention if those targets associate with the Na,K-ATPase. Metastatic cells have the ability to migrate to other tissues or organs thus spreading the cancer.

Beyond the targeting aspect, the targeting moiety portion of the EDC of the invention can bring other advantages. For example, the targeting moiety can facilitate transport across the blood brain barrier (for example, antibodies can facilitate this transport [Sci Transl Med 3, 84ra43 (2011) and Sci Transl Med 3, 84ra44 (2011)]); the targeting moiety can increase the in vivo half-life of a therapeutic agent (three IgG subclasses have half-lives of about 20 days in humans); and/or the targeting moiety can increase the solubility of the agent in aqueous solutions such as the circulatory structure or pharmaceutical diluents.

In various embodiments of the EDC of the invention, the targeting moiety on the EDC of the invention can act to: (i) keep the agents of the invention near or on the target for a prolonged period of time (depending on its binding affinity), (ii) prevent or retard degradation by lysosomal enzymes, because a non-internalizing targeting moiety is not internalized into the cells by a receptor/antigen type of endocytosis and so does not reach the lysosomal system, and (iii) prevent uptake intracellularly by fluid phase endocytosis in a manner dependent linearly on its extracellular concentration. A non-internalizing characteristic of a targeting moiety can be determined experimentally by one skilled in the art. By way of example, non-internalizing antibodies are those that interact with antigens and epitopes present at the surface of target tissue extracellular constituents such as those of the extracellular matrix and do not enter the lysosome where they can become degraded.

The antibodies in the EDC of the invention can include various monoclonal antibodies, polyclonal antibodies, modified antibodies, chimeric antibodies or improved antibodies as described in the definitions provided above. For example, modern alternative strategies now allow for the production of fully humanized antibodies to reduce the immunogenicity of the antibody. In addition, smaller antibody fragments can be engineered, including antigen binding Fabs, Fvs, scFv, and minibodies, and the antibody can also be enhanced to increase the antibody's affinity, stability, and expression level (see Nat Med. 2003 January; 9(1):129-34).

In an alternative embodiment of the invention, the targeting moiety of the EDC of the invention is not an antibody but is instead a peptide or protein or peptidomimetic that is the functional equivalent, in terms of targeting, of an antibody. For example and without limitation, the antibody can be replaced by any of a number of small and robust non-immunoglobulin “scaffolds” that can be equipped with prescribed binding functions using the methods of combinatorial protein design. Such scaffolds are described in various reviews (see, e.g. “Engineered protein scaffolds as next-generation antibody therapeutics” in Curr Opin Chem Biol. 2009 June; 13(3):245-55 and “Engineered affinity proteins for tumour-targeting applications” in Biotechnol Appl Biochem. 2009 May; 53(Pt 1):1-29).

In another alternative embodiment of the invention, the targeting moiety of the EDC of the invention is not an antibody but is instead a DNA, RNA, or oligonucleotide mimetic that is the functional equivalent, in terms of targeting, of an antibody. For example, SELEX methods can be used to identify DNA or RNA or modifications thereof with prescribed binding functions. Aptamers are polymers of RNA or DNA oligonucleotides or modifications thereof that are isolated by the systematic evolution of ligands like the exponential enrichment SELEX process (see Hicke and Stephens, 2000, “Escort Aptamers: A Delivery Service for Diagnosis and Therapy,” J. Clin. Invest., 106(8), pp. 923-928).

Typically, the targeting moiety (or other binding or targeting moiety) will be purified to greater than 95% by weight (as determined, for example, by the Lowry method), and often to more than 99% by weight prior to use in forming an EDC of the invention. Ordinarily, the targeting moiety will be prepared by at least one purification step. Once a targeting moiety of interest is available, it can be linked to a therapeutic agent by any of a variety of linkers and linker technologies, as discussed in the following section.

Illustrative targeting moieties for EDC of the invention include antibodies to CD147 such as Gavilimomab or Ziralimumab. Additional targeting moieties for EDC of the invention include antibodies specific to EpCAM such as Adecatumumab, Citatuzumab bogatox or Catumaxomab, antibodies specific to CD44 such as Bivatuzumab mertansine, antibodies specific to RANKL such as Denosumab, antibodies specific to IGF-1 Receptor such as Figitumumab or Robatumumab, integrins such as MEDI-522 also known as Vitaxin, ReoPro, Tysabri, and Ambegrin, and other antibodies that have high affinities to extracellular epitopes of extracellular targets that complex with the Na,K-ATPase.

Thus, there is a wide variety of targets and targeting moieties that can be used to make EDC of the invention. Targeting moieties of particular interest are those that target disease specific extracellular targets that are located in close proximity to the Na,K-ATPase. The EDC of the present invention include EDC comprising targeting agents (antibodies and/or drugs) that are not specific enough, as single or stand-alone agents, to be used for therapeutic purposes. Such agents now find therapeutic application in EDC of the invention.

Antibody targeting moieties for many of these targets already exist or can be made by one of ordinary skill in the art in view of the disclosure herein. As demonstrated herein, EDC with antibodies targeting receptors such as CD147 (EDC2), CD44 (EDC3), CD98 (EDC4), CD87, CD230, and CD56 find useful applications as anticancer agents in oncology, and numerous other targets (and corresponding antibodies) suitable for EDC of the invention exist (e.g., CD29, CD71, CD166, and EpCAM, for example). For inflammation, targets (and corresponding antibodies) suitable for EDC of the invention include CD28, PLA2, and T-Cell Receptor. For diabetes, targets (and corresponding antibodies) suitable for EDC of the invention include PE-NMT, insulin receptor (CD220), and angiotensin. Moreover, these indications are merely illustrative of the diverse indications for which EDC of the invention provide important new therapeutic agents.

Thus, the EDC of the invention can utilize any of a wide variety of targeting moieties that bind targets in close proximity to the Na,K-ATPase.

### IV. Linkers

To form an EDC of the invention, a therapeutic agent is coupled to the targeting moiety via a stable linker. In various embodiments, the linker includes a least one glycoside (sugar) residue, which is typically attached to the drug of the EDC. The linker, if conceptualized as a discrete entity instead of part of an EDC, is a monofunctional or multifunctional moiety that can be used to link one or more drugs to an antibody to form an EDC. EDC can be conveniently prepared using a linker having reactive functionality for binding to the drug and to the antibody. For example, a cysteine thiol, or an amine, e.g. N-terminus or amino acid side chain such as lysine, of an antibody can form a bond with a functional group of a linker reagent or drug-linker reagent (composed of the drug and linker).

Linkers for use in the EDC preferred for the methods of the present invention are generally comprised of polyethylene glycol (PEG) and one or more glycosides. In various embodiments, the PEG portion of the linker contains from 2 to 36 glycol units. In various embodiments, the PEG portion of the linker contains 24 glycol units. In various embodiments, the linker contains a single glycoside. In various embodiments, a glycoside is initially attached the agent at various sites on the agent and these agent-glycosides are tested for activity. In various embodiments, the glycoside portion of the linker contains a 3-amino-riboside, 4-amino-riboside, 3-amino-xyloside, and/or 4-amino-xyloside. In various embodiments, the glycoside portion of the linker is required for maximal activity of the EDC. In various embodiments, the agent is attached to the linker via a glycoside, and the glycoside is selected from the group consisting of 3-amino-riboside, 4-amino-riboside, 3-amino-xyloside, and 4-amino-xyloside, and the PEG portion of the linker is attached to the amino group of the glycoside. Typically, in the manufacture of an EDC in accordance with the methods of manufacture of the invention, a drug-linker reagent is formed first and then the drug-linker reagent is covalently coupled to the antibody to form the EDC. When the agent is digoxigenin, progesterone, or scillarenin, the glycoside is either 4-amino-riboside or 4-amino-xyloside. When the agent is ouabain, the glycoside is 3-amino-riboside or 3-amino-xyloside.

The linkers employed in the EDC of the invention are stable. After administration, the EDC is stable and remains intact, e.g. the targeting moiety remains linked to the agent via the linker. The linkers are stable outside the target cell and remain uncleaved for efficacy. An effective linker will: (i) maintain the specific binding properties of the antibody; (ii) allow delivery of the conjugate or agent; (iii) remain stable and intact, e.g. not cleaved, for as long as the antibody and/or agent remains stable and intact; and (iv) maintain a cytotoxic, cell-killing effect or a cytostatic effect of the agent while the EDC is intact. By way of example, stable linkers are those that, when in an EDC of the invention, show minimal (e.g., less than 10%) cleavage while present in the circulatory structure, at the surface of target tissue, at the surface of target cell, or in the extracellular matrix for a period of at least 4 to 8 hours or longer, such as 8 to 24 hours, or 1 to 10 days or longer; non-cleavable linkers are stable in these conditions for longer periods, including periods as long as 20 days or longer (Durcy, L. et. al. Bioconjugate Chem. 2010, 21, 5-13).

The linkers employed in the EDC of the invention can be conveniently produced in two stages. In the first stage, a glycoside that contains an active nucleophile such as a free primary amine is attached to a steroidal agent such as digitoxigenin or scillarenin. In the second stage, a bifunctional PEG linker is attached to the glycoside's amine. This method is advantageous in that it allows various combinations of glycosides and different linker lengths to be added in succession. In addition, glycosides have been shown to have certain advantages when employed in the linker portion of the invention.

A stable linker forms a covalent bond between the therapeutic agent and a targeting moiety such that, when attached, the agent and targeting moiety can bind and act on their respective targets. While a stable linker can simply be a covalent bond formed between reactive sites on the targeting moiety and the agent, the stable linkers of the invention typically include a linker spacer group, e.g., a repeating series of ethylene glycol units and an amino-glycoside. To attach a targeting moiety to an agent through a linker, one utilizes complementary reactive groups. For example, accessible sulfhydryl groups on a targeting moiety can react with active maleimide groups to form stable thioether linkages. An additional example is accessible amines on an agent can react with succinimide esters to form stable amide bonds. Bifunctional linkers which have maleimides on one end and succinimide esters on the other can be used to link the drug to the antibody. As illustrated in the examples below, an EDC can be conveniently prepared by linking an amino glycoside to a hydroxyl group of a steroid drug forming an O-glycosidic linkage. Then an NHS-PEG-maleimide reagent is linked to the amino group of the amino glycoside to form a “linker-reagent”. Finally the maleimide in the linker-reagent is covalently attached to a cysteine moiety in the antibody.

Thus, distinct chemical linkers (as opposed to a single covalent bond) are typically used in EDC. Linkers of this type are typically linear chains of atoms or polymers consisting of one or more “linker spacer groups” with two “ends” that contain functional groups that can serve as linking reagents to connect the targeting moiety and/or therapeutic agent to the linker covalently. Suitable linkers can include a wide variety of functional groups and moieties, including but not limited to substituted or unsubstituted alkyl, substituted or unsubstituted heteroalkyl, substituted or unsubstituted aryl, aldehydes, acids, esters and anhydrides, sulfydryl or carboxyl groups, such as maleimido benzoic acid derivatives, maleimidocaproic acid derivatives, and succinimido derivatives, or may be derived from cyano bromide or chloride, succinimidyl esters or sulphonic halides and the like.

The linker can impart beneficial properties to an EDC of the invention in addition to physically linking the targeting moiety and the drug. The linker can be used to minimize agent self-association or aggregation of the EDC caused by the agent. The linker may also improve the therapeutic efficacy of the EDC. The linker may also improve the pharmacokinetics of the EDC. When the targeting moiety is linked to the therapeutic agent, the linker group may have several other functions, such as making the compound of the invention more bio-resistant, more bio-compatible, less immunogenic, less toxic, and/or more stable while in the circulatory structure or more stable to other types of destruction or elimination or to make it non-cleavable. Thus in certain embodiments, the stable or non-cleavable linker maintains the attachment of the targeting moiety to the therapeutic agent under physiological conditions, but may also have beneficial therapeutic effects as well.

An example of a stable, non-cleavable linker is the polyalkylene glycol linker. Another example of a stable, non-cleavable linker is a glycoside attached to a polyalkylene glycol linker. Polyalkyleneglycol linkers are linear chains that have at least two, and typically more than two, alkylene moieties linked together by oxygen in the form of an ether linkage. Glycoside attached polyalkylene glycol linkers are linear chains of polyalkylene glycol that has a sugar, such as an aminoglycoside, attached. The alkylene groups can be substituted, but typically are unsubstituted, and can comprise any desired number of alkylene units, but typically at least 2 or no more than 100 such units, e.g., ethylene, propylene, hexylene, and the like. In one embodiment, the linker is comprised of 24 repeating ethyleneglycol units making a PEG24-type linker. This linker would be approximately 90-100 angstoms long depending on the reactive groups attached to either end. Generally, the linker length will be in the range of about 50 to about 500 Angstroms or about 50 to about 200 Angstroms. In one embodiment, the linker is comprised of a sugar. In various embodiments, as described above; the linker contains an amino sugar. The polyalkyleneglycol residue can comprise repeating alkylene units which are all the same or which vary in length and/or substitution. In various embodiments, the linker of the EDC of the invention is constructed using a (PEG)36 bifunctional linker. In a particular embodiment, the linker of the EDC of the invention is constructed using SM(PEG)24 from Thermo Scientific. In a particular embodiment, the linker of the EDC of the invention is constructed using an amino-glycoside. In a particular embodiment, the linker of the EDC of the invention is constructed using 3-amino-riboside, 4-amino-riboside, 3-amino-xyloside, and/or 4-amino-xyloside.

When polyethyleneglycol (PEG) and one or more glycosides are used to link the targeting moiety to the drug, the EDC may be capable of withstanding attacks by the immune system. Adding PEG to proteins or small molecules has been shown to improve therapeutic efficacy of some protein or small molecule therapeutics (see PEGylated Protein Drugs: Basic Science and Clinical; Applications Series: Milestones in Drug Therapy Veronese, Francesco M. (Ed.)2009 and Advanced Drug Delivery Reviews Volume 55, Issue 10, 26 Sep. 2003, Pages 1261-1277, incorporated herein by reference). PEG can therefore increase the serum half-life and reduce antigenicity.

When a sugar, such as an aminoglycoside, is used to link the antibody to the drug via a polyalkylene glycol, the EDC may have an enhanced ability to withstand attacks by the immune system relative to EDC lacking such a sugar. In a particular embodiment, the preferred glycosides of the linker are 3-amino-riboside, 4-amino-riboside, 3-amino-xyloside, and/or 4-amino-xyloside. Those of skill in the art will appreciate that there are numerous glycosides that could be us that contain primary amines or nucleophiles which could be used to attached the glycoside to the other portion of the linker. Preferred glycosides are those that are not substrates for naturally occurring enzymes. Adding sugars to proteins or small molecules has been shown to improve therapeutic efficacy of antibodies or small molecule therapeutics (see Nature Reviews Drug Discovery 8, 226-234 (March 2009) and see Essentials of Glycobiology. 2nd edition. Cold Spring Harbor Laboratory Press; 2009). Sugars can therefore increase solubility thus reducing aggregation and reduce antigenicity.

Glycosides of the linker of the invention can include D or L of hexose, pentose, deoxyhexose, deoxypentose, deoxy-halohexose, deoxy-halopentose, deoxy-aminopentose, deoxy-aminohexose, tetrose, heterosugar, carboxysugar, a derivative of the aforementioned sugars, a disaccharide derived from at least one of the aforementioned sugars, or a polysaccharide derived from at least one of the aforementioned sugars. Suitable sugars include, e.g., L-ribose, D-ribose, L-fucose, D-fucose, 2-deoxy-D-galactose, 3-deoxy-D-glucose, 6-deoxy-D-glucose, 2-deoxy-2-fluoro-D-glucose, 6-deoxy-6-fluoro-D-glucose, L-lyxose, D-lyxose, L-rhamnose, L-allose, D-allose, L-altrose, D-altrose, L-galactose, D-galactose, L-xylose, D-xylose, D-gulose, L-mannose, D-mannose, L-idose, D-idose, L-mycarose, 6-keto-D-galactose, L-arabinose, D-arabinose, N-acetyl-D-galactosaminose, melibiose, lactose, maltose, D-galacturonose, L-talose, D-talose, 6-deoxy-6-azo-D-mannose, L-glucose, D-glucose, and amino-glycosides thereof.

While the order of attachment of the antibody, linker portions, and drug can be varied in the manufacture of an EDC, typically the manufacturing process proceeds by first synthesizing a drug, then attaching the sugar portion of the linker, then attaching the PEG portion of the linker and finally attaching antibody. As those of skill in the art will appreciate, an antibody may present multiple sites for covalent attachment of the drug-linker reagent (or linker). By appropriate modification of the coupling conditions, one can make preparations of EDC in which the average number of drugs per antibody varies according to the conditions employed. In various methods of the invention, this average number is important in achieving maximal beneficial therapeutic effect of the EDC, as discussed below.

To form an EDC of the invention, a therapeutic agent is coupled to the targeting moiety via a stable linker. The linker attaches the targeting moiety to an agent through one or more covalent bond(s), and, because stable linkers are required, the linker typically does not include a disulfide group or ester group. The linker is a functional or multifunctional moiety which can be used to link one or more agents and a targeting moiety to form an EDC of the invention. EDC can be conveniently prepared using a linker having reactive functionality for binding to the targeting moiety. For example, a cysteine thiol, or an amine, e.g. N-terminus or amino acid side chain such as lysine, of an antibody type targeting moiety can form a bond with a functional group of a linker reagent or drug-linker reagent. In some embodiments of the invention, a drug-linker agent is synthesized and then coupled to an antibody or other targeting moiety to form an EDC of the invention. As illustrated in Example 1, below, useful drug-linker agents of the invention include those in which the drug is a steroid, such as the aglycon of a cardiac glycoside and the linker is a glycoside linked to a PEG spacer arm. In various embodiments of the invention the linker or a spacer arm in the linker is at least 50 Angstroms long, or at least 75 Angstroms, or at least 95 Angstroms long. In one embodiment, the linker is at least 95 Angstroms long but less than 200 Angstroms long.

The linkers employed in the EDC of the invention are stable. After administration, the EDC is stable and remains intact, e.g. the targeting moiety remains linked to the agent via the linker. The linkers are stable outside the target cell and remain uncleaved for efficacy. An effective linker will: (i) maintain the specific binding properties of the antibody; (ii) allow delivery of the conjugate or agent; (iii) remain stable and intact, e.g. not cleaved, for as long as the antibody and/or agent remains stable and intact; and (iv) maintain a cytotoxic, cell-killing effect or a cytostatic effect of the agent while the EDC is intact. Stability of the EDC may be measured by standard analytical techniques such as mass spectroscopy, HPLC, and the separation/analysis technique LC/MS.

A stable linker forms a covalent bond between the therapeutic agent and a targeting moiety such that, when attached, the agent and targeting moiety can bind and act on their respective targets. While a stable linker can simply be a covalent bond formed between reactive sites on the targeting moiety and the agent, the stable linkers of the invention typically include a linker spacer group and a glycoside. To attach a targeting moiety to a drug-linker reagent, one utilizes reactive groups. For example, accessible sulfhydryl groups on a targeting moiety, can react with active maleimide groups to form stable thioether linkages.

The linker can impart beneficial properties to an EDC of the invention in addition to physically linking the targeting moiety and the drug. The linker can be used to minimize agent self-association or aggregation of the EDC caused by the agent (for example, by introducing a hydrophilic methoxytriethylene glycol chain onto the doxorubicin portion of the branched peptide linkers, aggregation was greatly reduced in the immunoconjugate products). The linker may also improve the therapeutic efficacy of the EDC (for example, increased linker stability between doxorubicin and BR64 monoclonal antibody resulted in increased efficacy and potency). The linker may also improve the pharmacokinetics of the EDC (for example, poly-ethylene glycol can increase serum half-life of antibodies and other molecules). A linker can also serve to increase the chemical reactivity of the agent or targeting moiety, and thus increase the coupling efficiency to the targeting moiety or agent. An increase in chemical reactivity can also facilitate the use of moieties, or functional groups on moieties, which otherwise would not be feasible to use. When the targeting moiety is linked to the therapeutic agent, the linker group may have several other functions, such as making the compound of the invention more bio-resistant, more bio-compatible, less immunogenic, less toxic, and/or more stable while in the circulatory structure or more stable to other types of destruction or elimination or to make it non-cleavable. Thus in certain embodiments, the stable or non-cleavable linker maintains the attachment of the targeting moiety to the therapeutic agent under physiological conditions, but may also have therapeutic effects as well.

The linkers used in the EDC of the invention are stable, and in various embodiments non-cleavable. In all embodiments, for the EDC of the invention to exert it's maximal therapeutic effect, the linker must remains intact, which requires the following: (i) the linkage between the targeting moiety and the therapeutic agent of the compound of the invention remains stable for a prolonged period of time in the circulatory structure, sufficient to allow the EDC to find and bind its target(s); (ii) the linkage remains stable while the EDC of the invention is stored under various conditions and temperatures for a prolonged period of time; and (iii) the stable characteristics of the EDC of the invention can be determined experimentally by one skilled in the art. By way of example, stable linkers are those that, when in an EDC of the invention, show minimal (e.g., less than 10%) cleavage while present in the circulatory structure, at the surface of target tissue, at the surface of target cell, or in the extracellular matrix for a period of at least 4 to 8 hours or longer, such as 8 to 24 hours, or 1 to 10 days or longer; non-cleavable linkers are stable in these conditions for longer periods, including periods as long as 20 days or longer (Durcy, L. et. al. Bioconjugate Chem. 2010, 21, 5-13).

An example of a stable, non-cleavable linker is the polyalkylene glycol linker attached to a glycoside via an amide bond. Polyalkyleneglycol linkers are linear chains that have at least two, and typically more than two, alkylene moieties linked together by oxygen in the form of an ether linkage. The alkylene groups can be substituted, but typically are unsubstituted, and can comprise any desired number of alkylene units, but typically at least 2 and no more than 5, or no more than 10, or no more than 25 or no more than 50, or no more than 100 such units, e.g., ethylene, propylene, hexylene, and the like. In one embodiment, the linker is comprised of 24 repeating ethyleneglycol units making a PEG24-type linker. This linker would be approximately 90-100 angstoms long depending on the reactive groups attached to either end. In one embodiment, the linker is comprised of a sugar. In one embodiment, the linker is comprised of an amino sugar. The polyalkyleneglycol residue can comprise repeating alkylene units which are all the same or which vary in length and/or substitution. In various embodiments, the linker of the EDC of the invention is constructed using a (PEG)36 bifunctional linker or spacer arm. In a particular embodiment, the linker of the EDC of the invention is constructed using SM(PEG)24 from Thermo Scientific.

Any substituent of one or more alkylene units will of course be selected such that the advantageous properties of the present invention are not substantially compromised. One skilled in the art will be able to make appropriate selections in view of the disclosure herein. Typically such substituents, if present, are hydroxyl, alkoxyl, or disubstituted amino moieties. When polyethyleneglycol (PEG) and one or more glycosides are used to link the targeting moiety to the drug, the EDC may be capable of withstanding attacks by the immune system. Adding PEG to proteins or small molecules has been shown to improve therapeutic efficacy of some protein or small molecule therapeutics (see PEGylated Protein Drugs: Basic Science and Clinical; Applications Series: Milestones in Drug Therapy Veronese, Francesco M. (Ed.)2009 and Advanced Drug Delivery Reviews Volume 55, Issue 10, 26 September 2003, Pages 1261-1277, incorporated herein by reference). PEG can therefore increase the half-life, reduce the requirement for frequent dosing, and reduce antigenicity as well.

Drugs may be coupled through linkers to site-specific locations engineered onto antibodies to make an EDC of the invention. For example, aldehyde tags can be made through the use of the formylglycine generating enzyme (FGE) which performs posttranslational modification converting cysteines within amino acid consensus sequences, also termed “sulfatase motifs” to the aldehyde-bearing residue formylglycine (FGIy). The motifs can be installed within heterologous proteins as a genetically encoded “aldehyde tag” for site-specific labeling with aminooxy- or hydrazide-functionalized probes (see Cell. 2003 May 16; 113(4):435-44; J Am Chem Soc. 2008 Sep. 17; 130(37): 12240-12241). Another example of modifying antibodies site selectively also is accomplished through cysteines and involves first identifying reactive thiol groups on the antibody surface using a Phage ELISA selection (see J Immunol Methods. 2008 Mar. 20; 332(1-2):41-52, and US Pat. App. Pub. No. US20080305044). Yet another method to site specifically label antibodies is through the use of unnatural amino acids incorporated into the antibody polypeptide chain (see Annu Rev Biophys Biomol Struct. 2006 35:225-49).

Polyfunctional linkers or dendrimers can be used in accordance with the methods of the invention so that multiple agents are attached to a single attachment site on the targeting moiety. In this way, multiple agents could be attached to a single specific site on the targeting moiety, which may be engineered as discussed above, or to multiple sites where reactive side chains are located. The number of drugs and linkers will be, in any event, at least one and an upper limit can be determined experimentally by one skilled in the art. An optimal number of linkers and drugs can be determined but this is not required. For example, one linker could have multiple therapeutic agents attached (dendrimer), and an EDC with multiple linkers could have a fraction of linkers free of therapeutic agent.

An optimal linker length may be determined by experimental measurements, for example by testing multiple linker lengths in the assay used to determine activity of the resulting EDC of the invention. For example, if linker length is too short (not allowing the drug and targeting moiety to reach their binding sites simultaneously), one can readily identify and correct the problem to provide an EDC of the invention. Typically, the linker length will be in the range of about 50 to about 500 Angstroms or about 50 to about 200 Angstroms. The linker length and composition is selected to ensure that the EDC remains stable in the circulatory structure where enzymes and other environmental substances may otherwise break it down and to reflect the distance from where the targeting moiety binds to its antigen and where the agent acts on its target. A wide variety of linkers that comply with these requirements are available or can be synthesized, which creates a very large class of EDC provided by the invention, particularly when one considers the wide variety of linkers, therapeutic agents and targeting moieties that can be employed in the EDC of the invention.

Thus, the EDC of the invention can utilize any of a wide variety of stable or non-cleavable linkers.

### V. Therapeutic Agents (Drugs)

A wide variety of therapeutic agents are suitable for use in the EDC of the invention. For example and without limitation, the therapeutic agent can be an agent with anti-tumor, anti-angiogenic, or anti-inflammatory therapeutic activity. Preferably, the site where the therapeutic agent (e.g., drug) is attached to the linker is at a position where the linker attachment only minimally interferes or does not interfere at all with the therapeutic agent's activity. The agents used in the EDC of the invention bind to or otherwise interact with targets that are associated with and in close proximity to, or are part of, the Na,K-ATPase. In various embodiments, the EDC of the invention have a steroid drug that binds the alpha subunit of the Na,K-ATPase.

Typically, the agent is a “non-internalizing therapeutic agent” that acts directly on the Na,K-ATPase, e.g., at the alpha subunit. In various embodiments of the invention, the agent or drug is an aglycon of a cardiac glycoside. In other embodiments of the invention, the agent acts to inhibit the interaction of the Na,K-ATPase and a cell surface pathway signaling protein associated therewith.

Those of skill in the art will appreciate that, while this disclosure illustrates the invention primarily with respect to EDC that contain a targeting moiety that targets a protein associated with the Na,K-ATPase and a therapeutic agent that targets the Na,K-ATPase, the invention also provides EDC of the converse type. In this embodiment of the invention, the targeting moiety's target is the Na,K-ATPase, and the agent acts on a protein associated with the Na,K-ATPase. Suitable antibodies and other targeting moieties specific for, and suitable therapeutic agents that bind to the alpha subunit of, the Na,K-ATPase are described in PCT Pub. No. 2011/021870 and PCT App. No. US2012/028585.

Agent loading refers to the average number of therapeutic agents per targeting moiety (e.g., antibody) in a EDC. Where each linker is linked to one therapeutic agent, the average number of therapeutic agents will equal the average number of linkers on the targeting moiety. Agent loading typically ranges from 1 to 8 agents per targeting moiety, if the targeting moiety is an antibody (Ab), e.g. where 1, 2, 3, 4, 5, 6, 7, and 8 therapeutic agents are covalently attached to the antibody. Thus, compositions of EDCs include collections of antibodies conjugated with a range of drugs, from 1 to 8. The average number of drugs per antibody in preparations of EDC from conjugation reactions may be characterized by conventional means such as mass spectroscopy, ELISA assay, electrophoresis, and HPLC. By ELISA, the averaged value of therapeutic agents in a particular preparation of EDC may be determined (Hamblett et al (2004) Clinical Cancer Res. 10:7063-7070; Sanderson et al (2005) Clinical Cancer Res. 11:843-852). However, it is not possible to identify the location of therapeutic agents and/or linkers conjugated to antibodies by ELISA based methods. In some instances, separation, purification, and characterization of homogeneous EDC (where the number of therapeutic agents is the same but the location on the antibody may be different) may be achieved by means such as reverse phase HPLC or electrophoresis.

Advantageous properties of EDC targeted agents that act on the Na,K-ATPase include: (i) increased potency because the targeting moiety can lower ED50 values; (ii) decreased toxicity because the agent will preferentially target the Na,K-ATPase associated with the targeting moiety's target as opposed to Na,K-ATPase generally; and/or (iii) increased pharmacokinetics because antibodies can increase the ½-life values of conjugated agents, antibodies can enhance the EFR (enhanced permeability and retention) effect on drugs, and antibodies can keep certain agents from being sequestered in proteins or lipids.

In many cases, therapeutic agents have side effects not related to the target of activation specified to the specific indication. Such side effects are complex phenomenological observations that have been attributed to a number of molecular scenarios including direct interaction with off-target binding [see Keiser, M J, et. al. Nature 462, 175 (2009), Blagg, Annu. Rep. Med. Chem. 41, 353 (2006), Toxicity and Drug Discov. Today 10, 1421 (2005)]. Subsequent activation of other targets or the same target when not in complex with certain specified proteins or environment, can lead to harmful side effects.

Several papers suggest that agents that target the Na,K-ATPase may have multiple physiological targets [see (BMC Structural Biology 2010, 10:12) and (Current Medicinal Chemistry, 2011, 18, 872-885) and (Frontiers in Bioscience 14, 2130-2148, Jan. 1, 2009)], indicating that such drugs may not be used without unacceptable side effects. In accordance with the methods and EDC of the invention, however, the ability of the Na,K-ATPase to complex with multiple proteins and the varying interactions and signals that can result, is exploited to provide a wide variety of drugs and therapeutic interventions. For example, the Na,K-ATPase association with a particular protein varies with cell and disease type, enabling EDC of the invention to be targeted to a wide variety of different cells and diseases.

Evidence for this important benefit of the invention includes the fact that cardiac glycosides are known to have negative or positive effects on the heart, brain and other organs. These effects are now known to depend on not only local concentration of the agent and type of agent, and cellular location of the Na,K-ATPase, but also on the proteins which the Na,K-ATPase associates or complexes with. Thus, by targeting the associated protein, one can direct an EDC of the invention only to a selected cell or disease cell type.

In addition to drugs known to target the Na,K-ATPase directly, such as the cardiac glycosides, there are drugs that, prior to the present invention, were believed to bind or affect proteins other than the Na,K-ATPase but now, in view of this disclosure, are known to effectuate their action via the Na,K-ATPase. One such example is the drug is glibenclamide, also known as glyburide, which is used to treat type 2 diabetes. Na,K-ATPase activity measured enzymatically and electrophysiologically was shown to be suppressed by glyburide (Ribalet, B. et al. J Gen Physiol. 1996 February; 107(2):231-41). Therefore, in other embodiments of the EDC of the invention, the agent is or is a derivative of glyburide. Another example is the agent progesterone that, in view of this disclosure, is believed to bind to the first external loop of the al-subunit of the Na,K-ATPase, which up-regulates phospholipid N-methyltransferase (see, Steroids. 2008 January; 73(1): 27-40). Both estrogen and progesterone exhibit significant inhibitory actions on the Na-K-ATPase pump in brain and in a number of other tissues as well (LaBella et al. Fed Proc 44:2806-2811).

Therefore, while in many embodiments of the EDC of the invention, the agent is a steroid that modulates the activity of the Na,K-ATPase, in other embodiments the agent is another compound. Examples of steroids include, digitoxigenin, scillarenin, and corticosteroids. Examples of such other compounds include, without limitation, compounds selected from the group consisting of progesterone, angiostatins, berberine, glyburide, 5-hydroxydecanoate, dimethyloxallyl glycine, and perillyl alcohol (see, Am J Cardiovasc Drugs; 7(2):135-41 (2007); Endothelium, 9: 3-10, (2002); Mol Cell Biochem December; 306 (1-2):231-7 (2007); J Gen Physiol 107(2):231-41 (1996); Nat.Genet. 18, 219-224 (1998); Mol Cell Biochem December; 306(1-2):231-7 (2007); Mol Cell Biochem 345:29-34 (2010); Steroids 73(1): 27-40 (2008)). In various embodiments, the agent is a channel blocker; in other embodiments, the agent is not a channel blocker.

In many embodiments, however, the drug in the EDC is an aglycon of a cardiac glycoside. Suitable cardiac glycosides, include, for example and without limitation, cardiac glycosides that have been approved for human use, cardiac glycosides in clinical trials, and cardiac glycosides described in PCT Pub. Nos. 2010/017480 and 2011/031870 and PCT App No. US2012/028585, incorporated herein by reference. For cardiac glycosides (and other agents), the term therapeutic index is used to compare actual therapeutic effects to off-target toxic side effects. An example of a potential anticancer drug is digitoxin, which has strong antitumor activities but high cardiotoxicity (see, Goldin. Digitalis and cancer. Lancet 1984; 1:1134). Therefore the drug alone has not been found effective as an anticancer agent in humans. Various cardiac glycoside agents such as digitoxin, digoxin or proscillaridin and their aglycons are useful in various embodiments of the invention. Such cardiac glycosides have been reported to exhibit cytotoxic activity against several different cancer types but at concentrations that have cardiotoxic effects in patients [see Felth, J. et. al. J. Nat. Prod. 72, 1969 (2009)]. Cardiac glycosides are a class of drugs derived from plants of the genera Digitalis, Strophanthus, and others, which have been prescribed for centuries to treat congestive heart failure and arrhythmias. In these conditions, cardiac glycosides bind to the Na,K-ATPase and inhibit its pumping activity. Studies performed over the last decade show that cardiac glycosides have activity as anti-cancer agents [Mijatovic et al. (2007) Biochim Biophys Acta 1776:32-57 and PCT Pub. No. 2010/017480].

Thus, in various embodiments of the invention, the therapeutic agent in the EDC is an aglycon of a cardiac glycoside. In one embodiment of the invention, the agent is scillarenin. In another embodiment, the agent is digitoxigenin. In another embodiment of the invention, the agent is a steroid containing a hydroxyl, amine or sulfur group at position C-3. In various embodiments of the invention, the steroid is a compound identified in PCT Pub. No. WO 2010/017480 or aglycon thereof Non-limiting examples of suitable steroid drugs include those of Formula I below as well as pharmaceutically acceptable esters, derivatives, conjugates, hydrates, solvates, prodrugs and salts thereof, or mixtures of any of the foregoing:

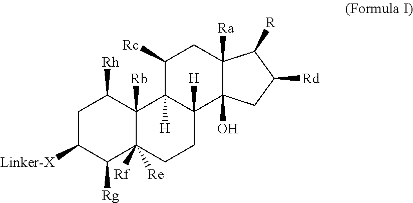

where the steroidal rings are either saturated, unsaturated or a combination thereof,

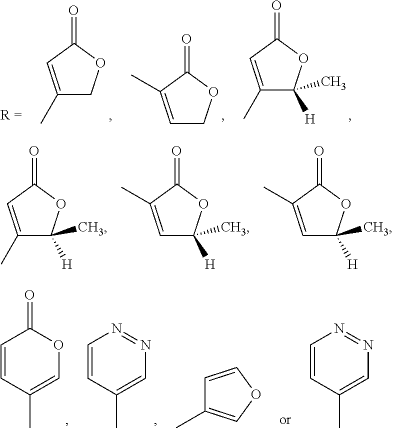

R can also be a side chain found on various corticosteroids such as CHOCH3, O, OH or a branched alkane. R^{a} is CH₃; R^{b} is CH₃, CH2OH, or CHO; R_{c}, is H, OH or CH₃COO; R_{d} is H, OH or CH₃COO; Rₑ is H or no group; R_{f} is H, OH or, when Rₑ is H or a C═C exists between the atoms joined to Rₑ, R_{f} and R_{g}, R_{f} is no group; R_{g} is H or, when Rₑ is H or a C═C exists between the atoms joined to Rₑ, R_{f} and R_{g}, R_{g} is no group; Rₕ is H or OH; X is O, S or N(OR′, SR′, NR′); and R′ is an alkyl or aryl group.

### VI. EDC Construction, Screening, and Specific Embodiments

The present invention provides EDC that comprise a targeting moiety linked to a therapeutic agent via a stable (and, in some embodiments, non-cleavable) linker and that do not require drug and targeting moiety dissociation for efficacy. The EDC of the invention comprise (i) a targeting moiety targeting a signaling pathway protein on the cell surface that interacts (is complexed) with and is in close proximity to the Na,K-ATPase, (ii) a stable or non-cleavable linker, and (iii) a therapeutic agent that targets the Na,K-ATPase or targets a site on the Na,K-ATPase or interacting protein at which binding blocks or otherwise modulates that interaction. The EDC of the invention also include EDC that comprise (i) a targeting moiety targeting the Na,K-ATPase, (ii) a stable or non-cleavable linker, and (iii) a therapeutic agent that targets a signaling pathway protein on the cell surface that interacts with and is in close proximity to the Na,K-ATPase or targets a site on the Na,K-ATPase at which binding blocks or otherwise modulates that interaction. In all of these instances, the EDC can be represented as follows: (targeting moiety)-(linker)-(therapeutic agent).

An EDC of the invention may be prepared by any of several routes, employing organic chemistry reactions, conditions, and reagents known to those skilled in the art, including: (1) reaction of a nucleophilic group or an electrophilic group of a targeting moiety with a bivalent linker reagent, to form antibody-linker intermediate, via a covalent bond, followed by reaction with an activated agent; and (2) reaction of a nucleophilic group or an electrophilic group of an agent with a linker reagent, to form drug-linker intermediate, via a covalent bond, followed by reaction with the nucleophilic group or an electrophilic group of a targeting moiety. Conjugation methods (1) and (2) may be employed with a variety of targeting moieties, agents, and linkers to prepare an EDC of the invention. Alternatively, the drug and linker may be synthesized in the form of a drug-linker agent that in turn is coupled to an antibody to generate an EDC of the invention (see Example 1, below). In this embodiment, the linker may be attached to the drug in multiple steps, using a subcomponent of the linker, such as a glycoside or a PEG spacer arm, in each step.

Nucleophilic groups on antibodies and other targeting moieties for example include, but are not limited to: (i) N-terminal amine groups, (ii) side chain amine groups, e.g. lysine, (iii) side chain thiol groups, e.g. cysteine, and (iv) sugar hydroxyl or amino groups where the antibody is glycosylated. Amine, thiol, and hydroxyl groups are nucleophilic and capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; (iii) aldehydes, ketones, carboxyl, and maleimide groups. Certain antibodies have reducible interchain disulfides, e.g. ,cysteine bridges. Antibodies may be made reactive for conjugation with linker reagents by treatment with a reducing agent such as DTT (Cleland's reagent, dithiothreitol) or TCEP (tris(2-carboxyethyl)phosphine hydrochloride; Getz et al (1999) Anal. Biochem. Vol 273:73-80; Soltec Ventures, Beverly, Mass.). Each cysteine disulfide bridge will thus form, theoretically, two reactive thiol nucleophiles. Additional nucleophilic groups can be introduced into antibodies through the reaction of lysines with 2-iminothiolane (Traut's reagent) resulting in conversion of an amine into a thiol.

EDC may also be produced by modification of the antibody to introduce electrophilic moieties, which can react with nucleophilic substituents on the linker reagent or drug. The sugars of glycosylated antibodies may be oxidized, e.g. with periodate oxidizing reagents, to form aldehyde or ketone groups which may react with the amine group of linker reagents or drug moieties. The resulting imine Schiff base groups may form a stable linkage, or may be reduced, e.g. by borohydride reagents to form stable amine linkages. In one embodiment, reaction of the carbohydrate portion of a glycosylated antibody with either galactose oxidase or sodium meta-periodate may yield carbonyl (aldehyde and ketone) groups in the protein that can react with appropriate groups on the drug (Hermanson, G. T. (1996) Bioconjugate Techniques; Academic Press: New York, p 234-242). In another embodiment, proteins containing N-terminal serine or threonine residues can react with sodium meta-periodate, resulting in production of an aldehyde in place of the first amino acid (Geoghegan & Stroh, (1992) Bioconjugate Chem. 3:138-146; U.S. Pat. No. 5,362,852). Such aldehyde can be reacted with a drug moiety or linker nucleophile.

Likewise, nucleophilic groups on a drug moiety include, but are not limited to: amine, thiol, hydroxyl, hydrazide, oxime, hydrazine, thiosemicarbazone, hydrazine carboxylate, and arylhydrazide groups capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; (iii) aldehydes, ketones, carboxyl, and maleimide groups.

In one embodiment, EDC of the invention are constructed by combinatorial synthesis and screening. Libraries or combinations of targeting moieties and agents linked through non-cleavable or stable linkers can be constructed and screened to identify specific EDC of the invention. In one embodiment, a single non-internalizing agent is linked to a library of targeting moieties and screened for a particular therapeutic effect to identify EDC of the invention. In one embodiment, a single non-internalizing targeting moiety is linked to a library of agents and screened for a particular therapeutic effect to identify EDC of the invention. In one embodiment, a library of non-internalizing agents is linked to a library of targeting moieties and screened for a particular therapeutic effect to identify EDC of the invention.

Close proximity targets that interact with the Na,K-ATPase in addition to those described herein may be identified by a number of methods in accordance with one aspect of the invention. In one such method, libraries of antibodies are conjugated to a drug that binds the Na,K-ATPase and are screened for various activities such as cytotoxicity, cell proliferation or expression or excretion of desired proteins or small molecules such as hormones. Because the conventional yeast two-hybrid system is not suitable for detecting interactions between membrane proteins, in another method of the invention, the split-ubiquitin membrane yeast two-hybrid system, which allows the interactions between an integral membrane protein and its partner(s) to be detected, is employed (Ohno et al., Am J Physiol Cell Physiol 2011 300: C1047-C1054). Close proximity proteins that form complexes with the Na,K-ATPase can also be identified in accordance with the invention by cross-linking studies or by testing their interactions in model systems or by removing one of the proteins in a model cell and running tests to analyze the outcome. When specific antibodies are available, immunoprecipitation of the target protein along with its noncovalent binding protein partners, e.g., coimmunoprecipitation (co-IP), has been a commonly used technique for identifying potential interacting proteins surrounding the target proteins. Co-IP methods have generated large-scale protein interaction data in yeast, mammalian, and many other organisms, and the validation of many of these results with orthogonal methods confirms the utility of these methods. Chemical cross-linking coupled with immunoprecipitation provides an alternative strategy for in vivo identification of protein-protein interactions which has been extensively reviewed. Cross-linking reactions can be carried out with intact cells and chemically “freeze” protein-protein interactions with stable covalent bonds that allow subsequent purification steps to be carried out under much harsher or more stringent conditions. Consequently, nonspecific binding can be reduced considerably. In addition, immunoprecipitation in conjunction with cross-linking is well suited for investigating the interactions of membrane proteins. Isolation and purification of membrane proteins usually requires use of detergents that can sometimes disrupt interactions among membrane proteins. Thus, stabilization of the complexes with cross-linkers prior to immunoprecipitation of membrane proteins significantly increases the chances of identification of the proteins bound to the antigens.

One skilled in the art knows how to crosslink associated or complex targets chemically. For example, protein-protein contacts can be revealed by incubating samples with cross-linking reagents. Complexes can be detected and isolated either by SDS-PAGE or by HPLC. Once isolated the targets can be digested and analyzed by mass spectroscopy to determine the fragments molecular mass and entered into databases or through computer algorithms, such as X!Link (see Lee et al., J. Proteome Res. 2007 October; 6(10):3908-17) to determine the actual proteins that form the close proximity targets. Employing chemical cross-linkers with added hydrophobicity coupled with immunoprecipitation is also a practical strategy for mapping protein-protein interactions in cell membranes (Tang et al., Mol. Bio Syst., 2010, 6, 939-947). Antibodies labeled with fluorescent dye pairs to both proteins can be used to confirm close proximity. The process of finding drugs and targeting moieties in close proximity to targets of this type would be as described above for targets that exist on the same protein.

An EDC of the invention is prepared by attaching the drug and the targeting moiety to one another via a stable and/or non-cleavable linker. Generally speaking, the linker should be no less than 50 Angstoms in length and is more typically 100 Angstroms in length but can be as long as 500 Angstroms in length. The EDC consisting of a drug, a linker and a targeting moiety all attached covalently to one another is then purified away from uncoupled drug, linker and targeting moiety using standard affinity, size exclusion, filtration, or other methods known to one skilled in the art.

Once a particular EDC is constructed, it may be screened using any of various methods known to those skilled in the art. Where the targets of the drug and targeting moiety are found on different proteins, these targets may only be in close proximity on certain cell types and thus multiple screens are conducted to determine the specificity and what cell types have the targets in close proximity. These include but are not limited to various in vitro and in vivo methods known in the art. EDC may be screened serially and individually, or in parallel under medium or high-throughput screening formats. The rate at which EDC may be screened for utility for prophylactic or therapeutic treatments of diseases or disorders is limited only by the rate of synthesis or screening methodology, including detecting/measuring/analysis of data.

Generally, in vitro screening is conducted first. For example, the cytotoxic or cytostatic activity of an EDC is first measured by: exposing test cells, e.g. mammalian cells having tumor-associated antigens or receptor proteins to the antibody of the EDC, in a cell culture medium; culturing the cells for a period from about 6 hours to about 5 days; and measuring cell viability (or other property). Cell-based in vitro assays are used to measure viability, e.g. proliferation (IC₅₀), cytotoxicity (EC₅₀), and induction of apoptosis (caspase activation) of the EDC.

For in vivo testing, transgenic animals and cell lines are particularly useful in screening antibody-drug conjugates (EDC) that have potential as prophylactic or therapeutic treatments of diseases or disorders involving overexpression of tumor-associated antigens and cell surface receptors, e.g. HER2 (U.S. Pat. No. 6,632,979). Screening for a useful EDC may involve administering candidate EDC over a range of doses to the transgenic animal, and assaying at various time points for the effect(s) of the EDC on the disease or disorder being evaluated. Alternatively, or additionally, the drug can be administered prior to or simultaneously with exposure to an inducer of the disease, if applicable.

EDC of the invention are provided in a variety of embodiments. As discussed above, the target of the targeting moiety of the EDC is typically a protein complexed with the Na,K-ATPase, and the target of the agent is the Na,K-ATPase. The targeting moiety and the therapeutic agent, when linked together, act synergistically in that the EDC is more effective in treating the condition of interest than either the targeting moiety or the drug used alone or in combination (as two separate, unliked agents). Thus, the target of the targeting moiety and the target of the agent of an EDC of the invention are both extracellular. EDC of the invention comprise a non-internalizing targeting moiety linked through a stable or non-cleavable linker to an agent. Thus, EDC of the invention do not require internalization to exert a therapeutic effect. The linker in an EDC of the invention is selected to allow both the targeting moiety and the agent to bind or act on their targets (when complexed together) without requiring linker cleavage. The targeting moiety and the therapeutic agent of the EDC thus bind simultaneously to create a desired therapeutic effect. Thus, the linker in the EDC is long enough to allow simultaneous binding of the targeting moiety and the agent when their respective targets are complexed together. The targeting moiety and the therapeutic agent of the EDC, linked by a stable or non-cleavable linker, act on different targets.

In various embodiments, the target antigens are on tumor cells, stromal cells, endothelial cells of tumors, cells such as macrophages, neutrophils, and monocytes, or are otherwise antigens associated with cancer, inflammatory reactions, diabetes, or pain. The EDC of the invention are administered to the patient, carried through the blood stream, and, when in the vicinity of a target cell or antigen, bind to a target antigen via the targeting moiety and simultaneously or contemporaneously bind to the target of the drug to create a desired therapeutic effect. In various embodiments, EDC of the invention comprise a targeting moiety that recognizes targets associated with metastatic diseased cells and complexed with the Na,K-ATPase in those diseased cells.

The invention provides EDC that comprise an agent that binds to the Na,K-ATPase to impair its normal function, a stable or non-cleavable linker, and a targeting moiety that recognizes a protein that complexes with the Na,K-ATPase ion transporter complex. The Na,K-ATPase is characterized by a complex molecular heterogeneity that results from the expression and differential association of multiple isoforms of its alpha-, beta- and gamma-subunits (see review in Am. J. Physiol. 275 (Renal Physiol. 44): F633-F650, 1998). The Na,K-ATPase belongs to a widely distributed class of P-type ATPases that are responsible for the active transport of a variety of cations across cell membranes. At present, as many as four different alpha-isoforms, three distinct beta-isoforms, and nine distinct gamma-isoforms have been identified in mammalian cells: The stringent constraints on the structure of the complex's isoforms during evolution and their tissue specific and developmental pattern of expression suggests that different Na,K-ATPase complexes have evolved distinct properties to respond to cellular requirements. Different isoforms of the alpha-subunit are expressed at different levels on different cell types and behave differently. The alpha-subunit contains the binding sites for cations, ATP, Src kinase, and various therapeutic agents. Some of the agents are derived from the cardiac glysoside class of molecules. Therefore in various embodiments of the invention, the alpha-subunit acts as the target for the agent of EDC of the invention and the agent is a cardiac glycoside or an aglycon of a cardiac glycoside.

Specifically, the cardiac glycoside class of molecules has been mainly used therapeutically in the treatment of cardiac failure, due to their anti-arrhythmic effects. Recently it was determined that this class of drugs also has anti-cancer activities, yet use as an anti-cancer drug has not yet been approved due to cardiotoxicity at levels required. Targeting this class of molecules away from the heart and toward cancer cells would thus be beneficial. Therefore, in one embodiment of the invention, cardiac glycoside derivatives or aglycons of cardiac glycosides are the drug of the EDC of the invention. In one embodiment, these EDC comprise a drug that is a member of the cardiac glycoside class of small molecules (or the aglycon portion thereof). In various embodiments of the invention, cardiac glycosides or related agents are attached through a stable linker to antibodies that target the drugs to cancerous tissue providing therapeutically useful EDC of the invention for treating cancer.

In one embodiment of the invention, the cardiac glycoside CEN-09-106 or its corresponding aglycon scillarenin is attached through a linker to an antibody that binds to a protein that forms a complex with the Na,K-ATPase. In one embodiment, the scillarenin aglycon is attached through a glycoside linker with a PEG spacer arm to an antibody that binds to a cell surface signaling pathway protein that forms a complex with the Na,K-ATPase. In another embodiment of the invention, the cardiac glycoside CEN-09-106 or scillarenin is attached through a linker to an antibody that binds to a protein that is in close proximity to the Na,K-ATPase. In another embodiment of the invention, the cardiac glycoside CEN-09-106 or scillarenin is attached through a PEG linker or spacer arm to an antibody that binds to a protein that forms a complex with the Na,K-ATPase. In another embodiment of the invention, the cardiac glycoside CEN-09-106 or scillarenin is attached through a PEG linker or spacer arm to an antibody that binds to a protein that is in close proximity to the Na,K-ATPase. In another embodiment of the invention, the cardiac glycoside CEN-09-106 or scillarenin is attached through a PEG24 linker or spacer arm to an antibody that binds to a protein that forms a complex with the Na,K-ATPase. In another embodiment of the invention, the cardiac glycoside CEN-09-106 or scillarenin is attached through a PEG24 linker or spacer arm to an antibody that binds to a protein that is in close proximity to the Na,K-ATPase.

The invention also provides EDC that comprise a targeting moiety linked to a cardiac glycoside that can be used as anti-inflammatory agent or to treat other diseases. Na,K-ATPase subunit isoform/modulator distribution and levels in the lungs of cystic fibrosis patients are distinct from those of a normal lung, and, so are a target for therapeutic agents against cystic fibrosis hyperinflammation. Cardiac glycosides that bind to the Na,K-ATPase can suppress hypersecretion of 1L-8 from cultured CF epithelial cells via specific inhibition phosphorylation of a NF-kappa B inhibitor (see Srivastava et.al. Proc. Natl. Acad. Sci. USA 2004, 101, 7693-7698, incorporated herein by reference). A review of the potential therapeutic uses of cardiac glycosides discusses obesity, kidney disease, migraines, epilepsy, dystonia, Parkinsonism (2007 Journal of Internal Medicine 261; 44-52). EDC of the invention can therefore be used to treat all of these conditions.

An illustrative lists of targets for the targeting moiety of the EDC of the invention that may interact with a Na,K-ATPase to modulate cell signaling pathways includes: CD147, LAT1, ASCT2, CD98, PrP, EpCAM, MCT1, Integrins (e.g., CD29 (integrin betal)), CD166, CD44 (HCELL), CD71, CD56, CD87, (TfR1), Sel-1, IGFR, c-MET, FGFR, PDGFR, GIuR2, Serotonin transporter, 5-HT1A Receptor, GABAA receptor, EAAT, TLR4, T-cell receptor, mTNF alpha (transmembrane), PLA2, RANKL, Insulin Receptor, PE-NMT, angiotensin II receptor, ATP-sensitive K channel, PE-NMT, angiotensin receptor, TNF-alpha, InsP3R, RS1, or α-klotho. Additionally, an illustrative lists of targets for the targeting moiety of the EDC of the invention that may interact with a Na,K-ATPase to modulate cell signaling pathways relevant to particular disease states includes: Cancer—CD147, LAT1, ASCT2, CD98, PRP, EpCAM, MCT1, Integrins (e.g., CD29 (integrin betal)), CD166, CD44 (HCELL), CD71, CD56, CD87, (TfR1), IGFR, c-MET, FGFR, PDGFR; Neurological disorders—CD147, PrP, MCT1, GluR2, Serotonin transporter, 5-HT1A Receptor, GABAA receptor, EAAT; Inflammatory disorders (e.g. Diarrhea, human inflammatory bowel diseases, rheumatoid arthritis)—TLR4, T-cell receptor, mTNF alpha (transmembrane), PLA2, RANKL; Diabetes/obesity—Insulin Receptor, PE-NMT, angiotensin II receptor, ATP-sensitive K channel; Cardiovascular disease-Integrin, PE-NMT, angiotensin receptor; Immunosuppression/immune-mediated disease/inflammation—TLR4, T-cell receptor, PLA2, TNF-alpha, InsP3R; Ischemic injury—Integrins, Macular degeneration: RSI; Aging/protein deficiency-α-klotho; Psoriasis—CD147. See, e.g., Molecular & Cellular Proteomics 4:1061-1071, 2005, Cancer Genomics and Proteomics May-June 2010 vol. 7 no. 3 157-169, Current Cancer Drug Targets, 2010, 10, 287-306, J Cell Physiol 208 (1): 23-38, 2006, J BIO CHEM 282 (45): 32792-32801, 2007, Physiol Rev 87: 593-658, 2007, Hum Mol Genet. 2011 Mar. 15; 20(6):1132-42, PLoS Biol. 2005 December; 3(12):e423, J. Neuroscience, Nov. 7, 2007, 27(45):12331-12340, Shakibaei and Mobasheri (2003) Histol Histopathol 18, 343-351, J Am Soc Nephrol 17: 1503-1520, 2006., Biochemistry. 2000 Dec. 5; 39(48):14877-83., Kidney International 78, 11.19-1127 (Dec. (1) 2010), Geriatr Gerontol Int 2010; 10 (Suppl. 1): S80-S87, Clinic Rev Bone Miner Metab (2008) 6:31-36, J. Biol. Chem. Vol. 274, No. 37, September 10, pp. 26287-26295, 1999, Arch Biochem Biophys. 1985 April; 238(1):315-24, BMC Structural Biology 2010, 10:12, Am J Physiol. 1988 September; 255(3 Pt 1):E347-52., J Pharmacol Exp Ther May 1988 245:664-672, Arterioscler Thromb Vase Biol. 2009 September; 29(9):1349-55. Epub 2009 Jun. 11, Am J Physiol Renal Physiol 290: F241-F250, 2006, N Engl J Med. 2010 Apr. 22; 362(16):1477-90, J. Pharm. Exp. Ther. JPET 285:835-843, 1998, J Gen Physiol. 1996 February; 107(2):231-41., Circulation. 2010; 122:A16963, Antiviral Research 79 (2008) 62-70, Hum. Mol. Genet. (2011) 20 (I): 90-103, Cho et al. J. Cellular Biochem. 2010 111:1252-1259, J. Neurosci., Jun. 24, 2009, 29(25):8143-8155, The Journal of Dermatology, Vol. 37, issue 12, pages 1053-1056, December 2010. A target for the targeting moiety of the EDC of the invention may be an extracellular target that interacts with Na,K-ATPase including where the extracellular target is in close proximity to the Na,K-ATPase or in complex with the Na,K-ATPase. Methods and reagents are provided for detecting a Na,K-ATPase interaction with (e.g., in close proximity with or in complex with) an extracellular target including illustrative targets as described above. Generally, these methods employ an antibody-drug conjugate (ADC) in which the antibody targets an extracellular target, such as a target involved in a cell surface signaling pathway of interest, and the drug targets the Na,K-ATPase and the two are linked by a stable or non-cleavable linker. This ADC together with appropriate controls (the antibody and the drug) are then tested in vitro and/or in vivo to determine if the ADC exerts a more potent and/or specific effect, such as cytotoxicity or inhibition of cell growth, proliferation, or differentiation, on one or more cell types of interest than either the antibody or drug alone. If the ADC is determined to exert a more potent or specific effect than either the antibody or drug alone, then the extracellular target (e.g. a cell surface protein) targeted by the antibody is determined to interact with the Na,K-ATPase in such cell type(s) of interest.

Thus, there are numerous targeting moiety targets for EDC of the invention in which the drug targets the Na,K-ATPase. EDC of the invention are useful, for example and without limitation, in cases where drugs are effective yet the specificity of the drug is such that off target side effects, low activity, poor pharmacokinetics, drug resistance (e.g. MDR mediated drug resistance) or other problems exist and where attaching the drug to a targeting moiety via a linker shows improves the therapeutic effect, as is the case, generally, for cardiac glycosides. EDC of the invention include those in which the agent and the targeting moiety work synergistically to enhance the desired therapeutic effect. EDC of the invention are also useful, for example and without limitation, in cases where better specificity for the agent is desirable, as is the case, again, with cardiac glycosides. This is the case when the agent's target may exist on many different types of normal cells and diseased cells but only as a complex or in close proximity with the targeting moiety's target on diseased or cells of interest.

EDC of the invention also include those that comprise a targeting moiety that recognizes a portion or subunit or isoform of the target antigen that are found preferentially on cells in or near the diseased or targeted tissue area. These target antigens include those that are mutated, differentially expressed or aberrantly glycosylated proteins (relative to those same proteins in normal tissue) in the diseased or targeted tissue area.

In various embodiments, EDC are generally useful for research purposes. The present invention arises from the discovery that the Na,K-ATPase associates with a wide variety of different proteins to modulate cell signaling pathways critical to a wide variety of cells and so implicated in a vast number of diseases. While this disclosure illustrates numerous such examples of Na,K-ATPase/protein interactions, there are undoubtedly many more. Convenient reagents for linking a cardiac glycoside to an antibody of interest; as well as EDC already linked to a cardiac glycoside, for example, are useful reagents provided by the invention that may be used for such studies.

The EDCs of the invention may also be used in methods for determining if a protein is complexed with a Na,K-ATPase on a cell surface. In some embodiments, the methods may comprise contacting the cell with an EDC of the invention and determining if the EDC has an effect on the cell (e.g., a reduction in cell viability or proliferation) that is different from an effect of contacting the cell with the targeting moiety or the therapeutic agent. The protein may be considered to be complexed with Na,K-ATPase where the EDC has an effect on the cell that is different from an effect of contacting the cell with the targeting moiety or the therapeutic agent.

In various embodiments of the invention, the EDC are generally useful for the treatment of cancer, cystic fibrosis, and many other diseases. Examples of diseases for cancer treatment include breast cancer, colorectal cancer, liver cancer, lung cancer, prostate cancer, ovarian cancer, brain cancer, and pancreatic cancer. Specifically, treatment for one of the following tumor types can be effected: B-cell lymphoblastic leukemia; T-cell lymphoblastic leukemia, lymphoma, including Hodgkin's lymphoma and non-Hodgkin's lymphoma, follicular lymphoma, Burkitt lymphoma, melanoma, ocular melanoma, cutaneous melanoma, colon adenocarcinomas, hepatocellular carcinomas, renal cell carcinoma, ovarian carcinoma, prostate adenocarcinoma, liver carcinoma, transitional cell carcinoma, pancreatic adenocarcinoma, lung carcinoma, breast carcinoma, and colon carcinoma.

In one embodiment, the invention provides an EDC composed of an antibody that binds CD 147 and a steroid drug that binds the alpha-subunit of the Na,K-ATPase. Example 2 illustrates such EDC. These EDC are useful in the treatment of cancer, including but not limited to small-cell lung cancer, non-small-cell lung cancer, colon cancer, pancreatic cancer, breast cancer, head and neck cancer, melanoma, and myeloma.

In one embodiment, the invention provides an EDC composed of an antibody that binds CD44 and a steroid drug that binds the alpha-subunit of the Na,K-ATPase. Example 3 illustrates such EDC. These EDC are useful in the treatment of cancer, including but not limited to non-small-cell lung cancer, and pancreatic cancer.

In one embodiment, the invention provides an EDC composed of an antibody that binds CD98 and a steroid drug that binds the alpha-subunit of the Na,K-ATPase. Example 4 illustrates such EDC. These EDC are useful in the treatment of cancer, including but not limited to non-small-cell lung cancer, pancreatic cancer, head and neck cancer, small cell lung cancer and myeloma.

In one embodiment, the invention provides an EDC composed of an antibody that binds CD87 and a steroid drug that binds the alpha-subunit of the Na,K-ATPase. Example 5 illustrates such EDC. These EDC are useful in the treatment of cancer, including but not limited to melanomas.

In one embodiment, the invention provides an EDC composed of an antibody that binds CD230 and a steroid drug that binds the alpha-subunit of the Na,K-ATPase. Example 6 illustrates such EDC. These EDC are useful in the treatment of prion disease, Alzheimer's disease, and cancer, including but not limited to non-small-cell lung cancer and melanoma.

In one embodiment, the invention provides an EDC composed of an antibody that binds CD56 and a steroid drug that binds the alpha-subunit of the Na,K-ATPase. Example 7 illustrates such EDC. These EDC are useful in the treatment of cancer, including but not limited to small-cell lung cancer.

In various embodiments, the invention provides methods of binding to an extracellular target that is not a NaK-ATPase comprising contacting a cell expressing the target with an EDC as disclosed herein.

In various embodiments, the invention provides methods of binding to an extracellular target that is not a NaK-ATPase comprising administering to a subject an amount of the EDC as disclosed herein effective to bind to the target.

Section VIII describes pharmaceutical formulations of the inventions and methods for administering them to treat disease and other medical conditions.

### VII. Pharmaceutical Formulations

The administration of the compounds or formulations according to the invention (e.g., compounds or formulations comprising the disclosed EDC) can be done by any of the administration methods accepted for the therapeutic agents and generally known in the art. These processes include, but are not limited to, systemic administration, for example by parenteral, oral, nasal, or topical administration. Parenteral administration is done generally by subcutaneous, intramuscular or intravenous injection, or by perfusion. In general, antibody based therapeutics are typically administered intravenously. The injectable compositions can be prepared in standard forms, either in suspension or liquid solution or in solid form that is suitable for an extemporaneous dissolution in a liquid. In one embodiment, parenteral administration uses the installation of a system with slow release or extended release that ensures the maintenance of a constant dose level. For intranasal administration, it is possible to use suitable intranasal vehicles that are well known to those skilled in the art. The oral administration can be done by means of tablets, capsules, soft capsules (including formulations with delayed release or extended release), pills, powders, granules, elixirs, dyes, suspensions, syrups and emulsions. This form of presentation is more particularly suited for the passage of the intestinal barrier.

The dosage for the administration of compounds or formulations according to the invention is selected according to a variety of factors including the type, strain, age, weight, sex and medical condition of the subject; the severity of the condition to be treated; the method of administration; the condition of the renal and hepatic functions of the subject and the nature of the particular compound or salt that is used and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations. For example, a normally experienced doctor will easily determine and prescribe the effective amount of the desired compound to prevent, disrupt or stop the progress of the medical condition that is to be treated. By way of examples, when given parenterally, the effective levels of the compounds according to the invention will be in the range of from about 0.002 to about 500 mg per kg of body weight, more particularly from about 0.02 mg to about 50 mg per kg of body weight and administered daily, weekly, or biweekly.

The compounds or formulations according to the invention can be administered in the form of single daily doses, or the total daily dosage can be administered in doses (e.g., divided doses) of two, three, four or more doses per day. Such doses may be administered intermittently, e.g. every week or every three weeks (e.g. such that the patient receives from about two to about twenty, e.g. about six doses, of the composition or formulation). An initial higher loading dose, followed by one or more lower doses, may be administered. An exemplary dosing regimen comprises administering an initial loading dose followed by a weekly maintenance dose. However, other dosage regimens may be useful. More specifically, the dosage can in some embodiments be similar in the range of 1-20 mgs/meter squared (mgs/m²) body surface area (bsa), and the doses can be administered weekly or every two weeks. For solid tumors the dosage may in some embodiments be higher, e.g., an initial dose in the range of 200 to 600 mgs/m² bsa or ˜1 to 20 mgs/kg (given, e.g., through a 120-minute intravenous infusion) and 150-350 mgs/m² or 1-10 mgs/kg (given through 60-minute intravenous infusion). Therefore the dosing range of the compounds according to the invention can be daily to weekly dosages of 1 mgs/m² to 500 mgs/m² bsa.

The compositions or formulations according to the invention can be sterilized and/or can contain one or more of: non-toxic adjuvants and auxiliary substances such as agents for preservation, stabilization, wetting or emulsification; agents that promote dissolution; and salts to regulate osmotic pressure and/or buffers. In addition, they can also contain other substances that offer a therapeutic advantage. The compositions are prepared, respectively, by standard processes of mixing, granulation or coating well known to those skilled in the art.

The compounds or formulations of the invention herein can be administered concurrently, sequentially, or alternating with the second drug or upon non-responsiveness with other therapy. Thus, the combined administration of a second drug includes co-administration, using separate formulations or a single pharmaceutical formulation, and consecutive administration in either order, wherein preferably there is a time period while both (or all) therapies simultaneously exert their biological activities. Multiple second drugs may be used in combination the compounds of the invention.

In another embodiment of the invention, articles of manufacture containing materials useful for the treatment of the disorders described above are provided. In one aspect, the article of manufacture comprises (a) a container comprising the compounds or formulations herein (preferably the container comprises the EDC and a pharmaceutically acceptable carrier or diluent within the container); and (b) a package insert with instructions for treating the disorder in a patient.

Also provided herein are methods for assessing the activity of an EDC of the invention. In one method, a targeting moiety can be used to recognize the target of the targeting moiety of the invention, but most preferably it is one of those disclosed herein. One of these methods is a method of assessing the presence of the antibody's target of the invention comprising subjecting patient tissue from tumors (such as lung cancer, but including all tumor types) to the targeting moiety alone of the EDC and analyzing binding by immunohistological methods which are known in the art. One method for assessing the activity of an EDC of the invention in tumor tissue comprises subjecting patient tissue from a tumor to fluorescent resonance transfer to determine whether the agent's and targeting moiety's targets are within close proximity. In this method the targeting moiety is labeled with one fluorophore and an agent target specific antibody (or similar) labeled with another fluorophore can absorb energy of a specific wavelength and re-emit energy at a different (but equally specific) wavelength. Another method for assessing the activity of the EDC in tumor tissue comprising subjecting patient tissue from tumors (such as lung cancer, but including all tumor types) treated with the EDC to FDG-PET imaging and then determining if the targeting moiety alone inhibits FDG uptake into the tissue. Inhibition of FDG uptake correlates with delayed tumor growth in this method by the EDC of the invention. Methods for carrying out the imaging and determining if FDG uptake is inhibited are known in the art.

Therapeutic EDC of the invention may be administered by any route appropriate to the condition to be treated. The EDC will typically be administered parenterally, e.g. infusion, subcutaneous, intramuscular, intravenous, intradermal, intrathecal, bolus, intratumor injection or epidural (Shire et al (2004) J. Pharm. Sciences 93(6):1390-1402). Pharmaceutical formulations of EDC are typically prepared for parenteral administration with a pharmaceutically acceptable parenteral vehicle and in a unit dosage injectable form. An EDC having the desired degree of purity is optionally mixed with pharmaceutically acceptable diluents, carriers, excipients or stabilizers, in the form of a lyophilized formulation or an aqueous solution (Remington's Pharmaceutical Sciences (1980) 16th edition, Osol, A. Ed.).

Acceptable parenteral vehicles, diluents, carriers, excipients, and stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethionium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). For example, lyophilized anti-ErbB2 antibody formulations are described in WO 97/04801, expressly incorporated herein by reference. An exemplary formulation of an EDC contains about 100 mg/ml of trehalose (2-(hydroxymethyl)-6-[3,4,5-trihydroxy-6-(hydroxymethyl)tetrahydropyran-2-yl]oxy-tetrahydropyran-3,4,5-triol; C₁₂H₂₂O₁₁; CAS Number 99-20-7) and about 0.1% TWEEN™ 20 (polysorbate 20; dodecanoic acid 2-[2-[3,4-bis(2-hydroxyethoxy)tetrahydrofuran-2-yl]-2-(2-hydroxyethoxy)et-hoxy]ethyl ester; C₂₆H₅₀O₁₀; CAS Number 9005-64-5) at approximately pH 6.

Pharmaceutical formulations of a therapeutic EDC may contain certain amounts of unreacted drug moiety (D), antibody (or other targeting moiety)-linker intermediate (Ab-L), and/or drug-linker intermediate (D-L), as a consequence of incomplete purification and separation of excess reagents, impurities, and by-products, in the process of making the EDC; or time/temperature hydrolysis or degradation upon storage of the bulk EDC or formulated EDC composition. For example, it may contain a detectable amount of drug-linker or various intermediates. Alternatively, or in addition to, it may contain a detectable amount of the un-linked free targeting moiety. An exemplary formulation may contain up to 10% molar equivalent of the agent of agent linker as it was determined by the in vitro cellular proliferation assays that in some cases the drug-linker conjugate less potent in cell killing than free drug.

The active pharmaceutical ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semi permeable matrices of solid hydrophobic polymers containing the EDC, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT™ (injectable. microspheres composed of lactic acid-glycolic acid copolymer and leuprolide acetate), and poly-D-(−)-3-hydroxybutyric acid.

The formulations to be used for in vivo administration must be sterile, which is readily accomplished by filtration through sterile filtration membranes.

The formulations include those suitable for the foregoing administration routes. The formulations may conveniently be presented in unit dosage form and may be prepared by any of the methods well known in the art of pharmacy. Techniques and formulations generally are found in Remington's Pharmaceutical Sciences (Mack Publishing Co., Easton, Pa.). Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Aqueous suspensions contain the active materials (EDC) in admixture with excipients suitable for the manufacture of aqueous suspensions. Such excipients include a suspending agent, such as sodium carboxymethylcellulose, croscarmellose, povidone, methylcellulose, hydroxypropyl methylcelluose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

The pharmaceutical compositions of EDC may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, an aqueous solution intended for intravenous infusion may contain from about 3 to 500 .mu.g of the active ingredient per milliliter of solution in order that infusion of a suitable volume at a rate of about 30 mL/hr can occur. Subcutaneous (bolus) administration may be effected with about 1.5 ml or less of total volume and a concentration of about 100 mg EDC per ml. For EDC that require frequent and chronic administration, the subcutaneous route may be employed, such as by pre-filled syringe or autoinjector device technology.

As a general proposition, the initial pharmaceutically effective amount of EDC administered per dose will be in the range of about 0.01-100 mg/kg, namely about 0.1 to 20 mg/kg of patient body weight per day, with the typical initial range of compound used being 0.3 to 15 mg/kg/day. For example, human patients may be initially dosed at about 1.0 mg EDC per kg patient body weight. The dose may be escalated to the maximally tolerated dose (MTD). The dosing schedule may be about every 3 weeks, but according to diagnosed condition or response, the schedule may be more or less frequent. The dose may be further adjusted during the course of treatment to be at or below MTD which can be safely administered for multiple cycles, such as about 4 or more.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

Although oral administration of protein therapeutics are generally disfavored due to poor bioavailability due to limited absorption, hydrolysis or denaturation in the gut, formulations of EDC suitable for oral administration may be prepared as discrete units such as capsules, cachets or tablets each containing a predetermined amount of the EDC.

The formulations may be packaged in unit-dose or multi-dose containers, for example sealed ampoules and vials, and may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described. Exemplary unit dosage formulations contain a daily dose or unit daily sub-dose, or an appropriate fraction thereof, of the active ingredient.

The invention further provides veterinary compositions comprising at least one active ingredient as above defined together with a veterinary carrier therefore. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

It is contemplated that the EDC of the present invention may be used to treat various diseases or disorders, such as cancer and autoimmune conditions in human or animal subjects. In one embodiment, the subject is a human. In another embodiment, the subject is a non-human animal (e.g dog, cat, horse, bird, etc.) Exemplary conditions or disorders include benign or malignant tumors; leukemia and lymphoid malignancies; other disorders such as neuronal, glial, astrocytal, hypothalamic, glandular, macrophagal, epithelial, stromal, blastocoelic, inflammatory, angiogenic and immunologic disorders.

The EDC compounds which are identified in the animal models and cell-based assays can be further tested in tumor-bearing higher primates and human clinical trials. Human clinical trials can be designed similar to the clinical trials testing efficacy. The clinical trial may be designed to evaluate the efficacy of an EDC in combination with known therapeutic regimens, such as radiation and/or chemotherapy involving known chemotherapeutic and/or cytotoxic agents (Pegram et al (1999) Oncogene 18:2241-2251). In one embodiment, the combination therapeutic agent is selected from Bevacizumab; Carboplatin; Cisplatin; Cyclophosphamide; Docetaxel injection; Doxorubicin; Etoposide; Etoposide Phosphate; Gemzar (gemcitabine HCL); Hycamtin (topotecan hydrochloride); Ifosfamide; Iressa (gefitinib); Irinotecan injection; Methotrexate injection; Mitomycin; Paclitaxel; Photofrin, QLT; Premetrexed; Procarbazine; Streptozocin; Tarceva (erlotinib); Vinblasine; Vincristine; and Vinorelbine tartrate.

Examples of cancer to be treated herein include, but are not limited to, carcinoma, lymphoma, blastoma, sarcoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, gastrointestinal stromal tumor (GIST), pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

For the prevention or treatment of disease, the appropriate dosage of an EDC will depend on the type of disease to be treated, as defined above, the severity and course of the disease, whether the molecule is administered for preventive or therapeutic purposes, previous therapy, the patient's clinical history and response to the antibody, and the discretion of the attending physician. The molecule is suitably administered to the patient at one time or over a series of treatments. Depending on the type and severity of the disease, about 1 μg/kg to 15 mg/kg (e.g., 0.1-20 mg/kg including, for example, 1 mg/kg to 15 mg/kg) of molecule is an initial candidate dosage for administration to the patient, whether, for example, by one or more separate administrations, or by continuous infusion. A typical daily dosage might range from about 1 μg/kg to 100 mg/kg (e.g., 1 mg/kg to 100 mg/kg) or more, depending on the factors mentioned above. An exemplary dosage of EDC to be administered to a patient is in the range of about 0.1 to about 10 mg/kg of patient weight.

An EDC of the invention may be combined in a pharmaceutical combination formulation, or dosing regimen as combination therapy, with a second compound having anti-cancer properties. The second compound of the pharmaceutical combination formulation or dosing regimen preferably has complementary activities to the EDC of the combination such that they do not adversely affect each other.

The second compound may be a chemotherapeutic agent, cytotoxic agent, cytokine, growth inhibitory agent, anti-hormonal agent, aromatase inhibitor, protein kinase inhibitor, lipid kinase inhibitor, anti-androgen, antisense oligonucleotide, ribozyme, gene therapy vaccine, anti-angiogenic agent and/or cardioprotectant. Such molecules are suitably present in combination in amounts that are effective for the purpose intended. A pharmaceutical composition containing an EDC may also have a therapeutically effective amount of a chemotherapeutic agent such as a tubulin-forming inhibitor, a topoisomerase inhibitor, or a DNA binder.

Other therapeutic regimens may be combined with the administration of an anticancer agent identified in accordance with this invention. The combination therapy may be administered as a simultaneous or sequential regimen. When administered sequentially, the combination may be administered in two or more administrations. The combined administration includes coadministration, using separate formulations or a single pharmaceutical formulation, and consecutive administration in either order, wherein there is a time period while both (or all) active agents simultaneously exert their biological activities.

In one embodiment, treatment with an EDC of the present invention involves the combined administration of an anticancer agent identified herein, and one or more chemotherapeutic agents or growth inhibitory agents, including coadministration of cocktails of different chemotherapeutic agents. Chemotherapeutic agents include taxanes (such as paclitaxel and doxetaxel) and/or anthracycline antibiotics. Preparation and dosing schedules for such chemotherapeutic agents may be used according to manufacturers's instructions or as determined empirically by the skilled practitioner. Preparation and dosing schedules for such chemotherapy are also described in Chemotherapy Service Ed., M. C. Perry, Williams & Wilkins, Baltimore, Md. (1992).

The anticancer agent may be combined with an anti-hormonal compound; e.g., an anti-estrogen compound such as tamoxifen; an anti-progesterone such as onapristone (EP 616812); or an anti-androgen such as flutamide, in dosages known for such molecules. Where the cancer to be treated is hormone independent cancer, the patient may previously have been subjected to anti-hormonal therapy and, after the cancer becomes hormone independent, the anti-ErbB2 antibody (and optionally other agents as described herein) may be administered to the patient. It may be beneficial to also coadminister a cardioprotectant (to prevent or reduce myocardial dysfunction associated with the therapy) or one or more cytokines to the patient. In addition to the above therapeutic regimes, the patient may be subjected to surgical removal of cancer cells and/or radiation therapy.

Suitable dosages for any of the above coadministered agents are those presently used and may be lowered due to the combined action (synergy) of the newly identified agent and other chemotherapeutic agents or treatments.

The combination therapy may provide an effect achieved when the active ingredients used together is greater than the sum of the effects that results from using the compounds separately. The effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, an effect may be attained when the compounds are administered or delivered sequentially, e.g. by different injections in separate syringes. In general, during alternation therapy, an effective dosage of each active ingredient is administered sequentially, e.g. serially, whereas in combination therapy, effective dosages of two or more active ingredients are administered together.

Also falling within the scope of this invention are the in vivo metabolic products of the EDC compounds described herein, to the extent such products are novel and unobvious over the prior art. Such products may result for example from the oxidation, reduction, hydrolysis, amidation, esterification, enzymatic cleavage, and the like, of the administered compound. Accordingly, the invention includes novel and unobvious compounds produced by a process comprising contacting a compound of this invention with a mammal for a period of time sufficient to yield a metabolic product thereof.

Metabolite products may be identified by preparing a radiolabelled EDC, administering it parenterally in a detectable dose (e.g. greater than about 0.5 mg/kg) to an animal such as rat, mouse, guinea pig, monkey, or to man, allowing sufficient time for metabolism to occur (typically about 30 seconds to 30 hours) and isolating its conversion products from the urine, blood or other biological samples. These products are easily isolated since they are labeled (others are isolated by the use of antibodies capable of binding epitopes surviving in the metabolite). The metabolite structures are determined in conventional fashion, e.g. by MS, LC/MS or NMR analysis. In general, analysis of metabolites is done in the same way as conventional drug metabolism studies well-known to those skilled in the art. The conversion products, so long as they are not otherwise found in vivo, are useful in diagnostic assays for therapeutic dosing of the EDC compounds.

Metabolites include the products of in vivo cleavage of the EDC where cleavage of any bond occurs that links the drug moiety to the antibody. Metabolic cleavage may thus result in the naked antibody, or an antibody fragment. The antibody metabolite may be linked to a part, or all, of the linker. Metabolic cleavage may also result in the production a drug moiety or part thereof. The drug moiety metabolite may be linked to a part, or all, of the linker.

In another embodiment, an article of manufacture, or “kit”, containing EDC and materials useful for the treatment of the disorders described above is provided. The article of manufacture comprises a container and a label or package insert on or associated with the container. Suitable containers include, for example, bottles, vials, syringes, or blister pack. The containers may be formed from a variety of materials such as glass or plastic. The container holds an EDC composition which is effective for treating the condition and may have a sterile access port (for example the container may be an intravenous solution bag or a vial having a stopper pierceable by a hypodermic injection needle). At least one active agent in the composition is an EDC. The label or package insert indicates that the composition is used for treating the condition of choice, such as cancer. For example, the cancer may be one which overexpresses one of the targets of the EDC of the invention. The label or package insert may also indicate that the composition can be used to treat cancer, wherein the cancer is not characterized by overexpression of one of the targets of the EDC of the invention. In other embodiments, the package insert may indicate that the EDC composition can be used also to treat hormone independent cancer, prostate cancer, colon cancer or colorectal cancer.

The article of manufacture may comprise a container with a compound contained therein, wherein the compound comprises an EDC of the present invention. The article of manufacture in this embodiment may further comprise a package insert indicating that the EDC can be used to treat cancer. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The following examples illustrate useful methods and EDC of the invention.

### EXAMPLES

Further advantages and characteristics of the invention will emerge from the following Examples, given by way of illustration and which are not to be construed as limiting. The invention may be practiced otherwise than as particularly described in the Examples. Numerous modifications and variations of the present invention are possible in light of the above teachings and, therefore, are within the scope of the appended claims following these Examples.

### Example 1

### Synthesis of Linker-Ready Therapeutic Agents, Preparation of EDCs, and Assessment of Biological Activity.

This example describes the synthesis of the “linker-ready” agent CEN010-105 in its thiol reactive form (Part A) and the conjugation of the steroid scillarenin to antibodies to form various EDCs of the invention (Part B). This example also describes various methods that can be used to assess EDC activity (Part C).

Part A Synthesis of “Linker-Ready” Agent

This example describes a synthetic protocol for attaching a steroid drug to a linker to produce a “linker-ready” agent that can be readily attached to an antibody, as described herein. By linking the amino acid cysteine to the thiol reactive form of the “linker-ready” agent, the capped “linker-ready” agent can also be used to investigate activity of a potential EDC breakdown product, as may be generated by EDC degradation by proteases in vivo.

CEN010-105 is a “linker-ready” scillarenin that comprises scillarenin, a linker and an active group used to form a covalent stable attachment to the antibody. The general synthetic steps for the preparation of CEN010-105 are as follows.

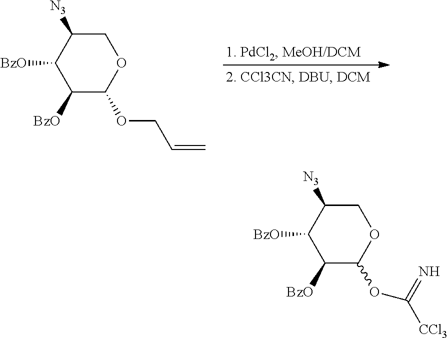

2,3-di-O-benzoyl-4-azido-4-deoxy-L-xylopyranoside-1-trichloroacetimidate. 1-Allyl-2,3-di-O-benzoyl-4-azido-4-deoxy-L-ribopyranoside (11.9 g, 28.1 mmol) was dissolved in dichloromethane/methanol (80 mL, 90:10) under argon, and PdCl₂ (0.5 g, 2.8 mmol) was added to the solution. The mixture was stirred overnight at room temperature, filtered through a pad of Celite and concentrated under reduced pressure. The residue was filtered through a pad of silica gel (hexane/EtOAc, 70:30). The resulting compound (8.38 g, 21.83 mmol) was dissolved in dry dichloromethane (170 mL) under argon. CCl₃CN (21.9 mL, 218.3 mmol) was added, followed by dropwise addition of DBU (1.63 mL, 10.91 mmol) at 0° C. The reaction was stirred for 1 h at 0° C. The solvent was removed under reduced pressure. The crude product was filtered through a pad of silica gel (hexane/EtOAc, 60:40 to 40:60) to afford 2,3-di-O-benzoyl-4-azido-4-deoxy-L-ribopyranosid-1-trichloroacetimidate as a yellow oil (9.7 g, 65%). The compound was carried forward without further purification. R_{f} 0.37 (silica gel, hexane/EtOAc, 80:20).

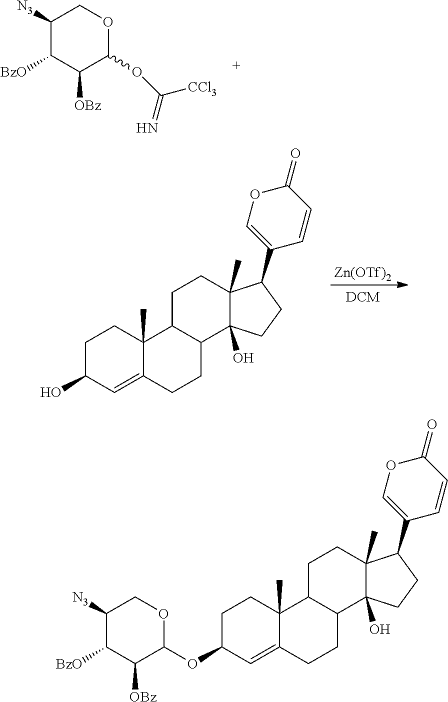

Scillarenin-2,3-di-O-benzoyl-4-azido-4-deoxy-L-xylopyranoside. 2,3-d i-O-benzoyl-4-azido-4-deoxy-L-xylopyranoside-1-trichloroacetimidate (0.483 g, 0.915 mmol) was added to a suspension of activated 4 Å molecular sieves (90 mg) in dry dichloromethane (15 mL) under argon at 0 ° C. Scillarenin (0.182 g, 0.474 mmol) was then added to the mixture. After 5 minutes, Zn(OTf)₂ (17 mg, 0.047 mmol) was added and the reaction mixture was stirred for an additional 30 minutes at 0° C. An additional amount of scillarenin (0.182 g, 0.474 mmol) was added. The reaction mixture was stirred for 30 minutes at 0° C. The reaction was quenched with few drops of Et₃N. The mixture was filtered and the solvent was removed under reduced pressure. The crude product was purified by flash chromatography (hexane/EtOAc, 75:25 to 50:50) to afford scillarenin-2,3-di-O-benzoyl-4-azido-4-deoxy-L-xylopyranoside as a white powder (0.521 g, 76%) R_{f} 0.35 (silica gel, hexane/EtOAc, 50:50). ¹H-NMR (300 MHz, CDCl₃) δ, 0.68 (s, 3H), 0.90-2.17 (m, 21 H), 2.39-2.44 (m, 1H), 3.47 (dd, 1H, J=12.0, 9.5 Hz, H-5b), 3.79-3.87 (m,1H, H-4), 4.17-4.22 (m, 2H, H-5a), 4.78 (d, 1H, J=6.8 Hz, H-1), 5.26 (dd, 1H, J=8.6, 6.8 Hz, H-2), 5.33 (s, 1H), 5.49 (dd, 1H, J=8.7 Hz, H-3), 6.22 (dd, 1H, J=9.7, 0.6 Hz), 7.18-7.19.(m, 1H), 7.33-7.39 (m, 4H), 7.47-7.53 (m, 2H), 7.80 (dd, 1H, J=9.7, 2.6 Hz), 7.92-7.97 (m, 41-1); ¹³C-NMR (75 MHz, CDCl₃) δ 16.7, 19.0, 21.4, 25.8, 28.7, 28.8, 32.4, 32.8, 35.2, 37.6, 40.8, 42.9, 48.4, 50.2, 51.2, 59.2, 63.1, 71.6, 72.9, 76.1, 85.2, 100.0, 115.5, 121.7, 122.8, 128.5, 128.6, 129.1, 129.5, 129.9, 130.1, 133.4, 133.6, 146.9, 147.6, 148.7, 162.5, 165.3, 165.7.

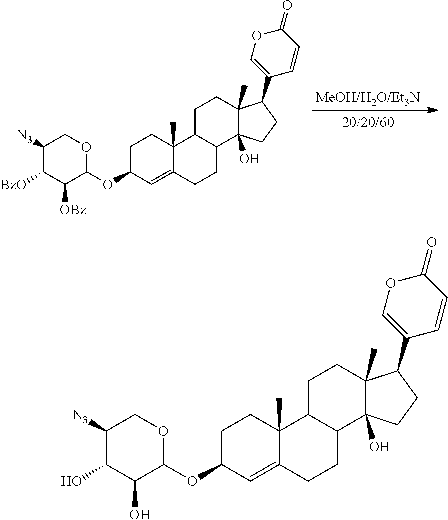

Scillarenin-4-azido-4-deoxy-L-xylopyranoside. Scillarenin-2,3-di-O-benzoyl-4-azido-4-deoxy-L-xylopyranoside (0.351 g, 0.468 mmol) was dissolved in methanol (21 mL). Et₃N (7 mL) and H₂O (7 mL) were added. The reaction mixture was stirred for 2 days at room temperature. The mixture was filtered and the solvent was stripped under reduced pressure. The crude product was purified by flash chromatography (CH₂Cl₂/MeOH, 98:2 to 95:5) to afford scillarenin-4-azido-4-deoxy-L-xylopyranoside as a yellow powder (40 mg, 24%) R_{f} 0.31 (CH₂Cl₂MeOH, 95:5); ¹H-NMR (300 MHz, CD₃OD) δ, 0.74 (s, 3H), 1.03-2.21 (m, 21H), 2.52-2.57 (m, 1H), 3.12-3.20 (m, 2H), 3.40-3.44 (m, 2H), 3.87-3.92 (m, 1H), 4.17-4.23 (m, 1H), 4.31 (d, 1H, J=7.7 Hz, H-1), 5.35 (s, 1H), 6.28 (dd, 1H, J=9.7, 0.8 Hz), 7.43 (d, 1H, J=1.5 Hz), 7.99 (dd, 1H, J=9.7, 2.6 Hz).

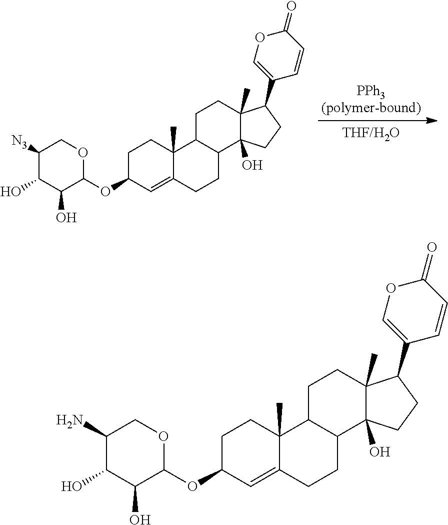 [SMILES: C[C@]12CC[C@H](OC3OC[C@H](N=[N+]=[N-])[C@@H](O)[C@@H]3O)C=C1CCC1C2CC[C@]2(C)[C@@H](c3ccc(=O)oc3)CC[C@]12O ; C[C@]12CC[C@H](OC3OC[C@H](N)[C@@H](O)[C@@H]3O)C=C1CCC1C2CC[C@]2(C)[C@@H](c3ccc(=O)oc3)CC[C@]12O]

Scillarenin-4-amino-4-deoxy-L-xylopyranoside. Scillarenin-4-azido-4-deoxy-L-xylopyranoside (1.61 g, 2.34 mmol) was dissolved in THF/H₂O (2.8 mL, 90:10). PPh₃ polymer-bound (79 mg, 3 mmol.g⁻¹) was added. The reaction mixture was stirred for 2 hours at 40° C. The mixture was then filtered and the solvent was removed under reduced pressure. The crude product was purified by flash chromatography (CH₂Cl₂/MeOH, 90:10 to 80:20) to afford scillarenin-4-amino-4-deoxy-L-xylopyranoside as a yellow powder (23 mg, 58%) R_{f} 0.2 (CH₂Cl₂MeOH, 80:20);.¹H-NMR (300 MHz, CD₃OD) δ, 0.74 (s, 3H), 1.06-2.19 (m, 21H), 2.52-2.57 (m, 1H), 2.75-2.86 (m, 1H, H-4), 3.14-3.24 (m, 2H, H-2, H-3), 3.64-3.72 (m, 1H, H-5b), 3.87-3.91 (m, 1H, H-5a), 4.19-4.24 (m, 1H), 4.36 (d, 1H, J=7.1 Hz, H-1), 5.38 (s, 1H), 6.28 (dd, 1H, J=9.7, 0.6 Hz), 7.42 (d, 1H, J=1.6 Hz), 7.99 (dd, 1H, J=9.7, 2.5 Hz); ¹³C-NMR (75 MHz, CD₃OD) δ 17.4, 19.6, 22.5, 26.8, 29.9, 30.1, 33.3, 33.6, 36.6, 38.8, 41.8, 43.5, 49.4, 51.7, 52.2, 75.3, 76.5, 78.9, 79.3, 79.8, 85.8, 103.7, 115.6, 123.4, 125.1, 148.4, 149.4, 150.5, 164.9.

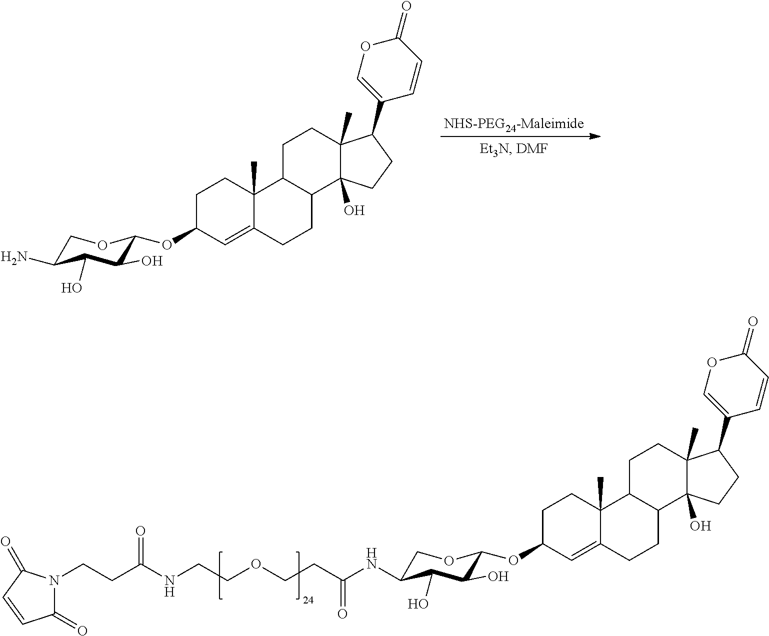

CEN010-105. To a solution of Scillarenin-4-amino-4-deoxy-L-xylopyranoside (18.5 mg, 0.0359 mmol) in DMF (1 mL) at room temperature was added NHS-PEG₂₄-Maleimide (50 mg, 0.0359 mmol). Then Et₃N (0.025 mL, 0.18 mmol) was added. The reaction was stirred at room temperature for 2 hours. The solvent was removed under reduced pressure. The crude material was purified by flash chromatography (CH₂Cl₂/MeOH, 95:5 to 80:20) to afford CEN010-105 as a yellow oil (48 mg, 75%) R_{f} 0.66 (CH₂Cl₂/MeOH, 80:20). HPLC analysis [Luna C18, 250×4.60 mm, 5 μm, 5% to 95% ACN over 32 minutes, 1 ml.min⁻¹] indicated a product which was >95% pure. HRMS-ESI (m/z): calculated for C₈₇H₁₄₇N₃O₃₅ [M+K^{+]}⁺: 1832.9452, found 1832.9777.

Part B Preparation of Immunoconjugates (EDCs and Control)

The EDCs described in Examples 2 through 9 and a control conjugate (contains antibody 4F12, mouse IgG kappa, which does not bind cell human cells) were prepared by the following method, involving reduction of antibody interchain disulfides. Briefly, antibody at concentrations between 1-10 mg/ml in PBS (20 mM sodium phosphate pH 7 and 150 mM NaCl) were reduced in the presence of 1 mM diethylenetriamine pentaacetic acid (DTPA) (MP Biomedical LLC) and 8 molar equivalents of tris(2-carboxyethyl)phosphine (TCEP) (cat. number: HR2-651, Hampton Research) at 37° C. for 2 hours then transferred to wet ice. Then 9.6 equivalents of CEN010-105 (“linker-ready” agent) were added and allowed to react for 30 min on ice. The reaction was quenched by the addition of 1.5 equivalents of L-cysteine over CEN010-105 and allowed to react 30 minutes at RT. The antibody conjugates were then separated from unconjugated CEN010-105 by repeated centrifugal concentration using Amicon Ultra 30,000 MWCO (Millipore, Billerica, Ma.) and DPBS buffer exchange. The conjugates were stored at 2-8° C. in PBS at concentrations ranging from 1-10 mg/ml.

Agent loading for EDCs (number of agents per antibody) with scillarenin as the agent is determined by the following method. Once the extinction coefficient of the agent is determined at a wavelength outside ±10 nm of 280 nm, this method can be used for any EDC comprising a steroid drug. The method entails measuring absorbance of the conjugates, the antibodies (Ab) and scillarenin (drug) at both 280 nm and, in the case of scillarenin, 299 nm. First, the absorbance of free antibody is measured at both 280 nm (A₂₈₀Ab) and 299 nm (A299Ab) to determine antibody constant [Constant Ab]. Next, the absorbance of free drug is measured at both 280 nm (A₂₈₀drug) and 299 nm (A₂₉₉drug) to determine drug constant [Constant Drug]. Finally, the absorbance of antibody drug conjugate is measured [A₂₈₀Conj and A₂₉₉Conj]. The antibody molar extinction coefficient at 280 nm=204,000 M⁻¹cm⁻¹. Scillarenin's molar extinction coefficient at 299 nm=5623 Agent loading of the conjugates is determined by solving the following equations.

[Constant Ab]=A₂₉₉Ab/A₂₈₀Ab

[Constant Drug]=A₂₉₉drug/A₂₈₀drug

A₂₈₀Ab*=A₂₈₀Conj-(A₂₉₉Conj—[Constant Ab]×A₂₈₀Conj)/([Constant drug]−[Constant Ab])

A₂₉₉drug*=A₂₉₉-[Constant Ab]×A₂₈₀Ab

Antibody concentrations=A₂₈₀Ab*/204,000 M⁻¹cm⁻¹

Drug concentration=A₂₉₉drug*/5623 M⁻¹ cm⁻¹

Drug loading=drug concentration/antibody concentration

A₂₉₉drug*=drug component of EDC

A₂₈₀Ab*=antibody component of EDC

Part C Cytotoxic Activity Assessment

Cells and culture conditions: Cell lines H460, HT29, A549, PANC-1, MB231, FaDu, H69 and H929 were obtained from the American Type Culture Collection (ATCC), Manassas, Va. The malignant melanoma cell line LOX IMVI was obtained from the DCTD Tumor Repository, National Cancer Institute, Frederick, Md., The cell lines were maintained in the recommended media formulations and subcultured every 3 to 4 days. To activate expression of certain targets to which the drug moiety bind and/or to form Na,K-ATPase complexes with certain proteins, cells can be cultured in recommended media plus additives such as phorbol esters, various growth factors and cytokines such as VEGF, fibroblast growth factors, human growth factors, interleukins, and tumor necrosis factors. In addition, cells can be cocultured with other cells like human fibroblasts. Additionally, microtiter plates can be coated with various proteins like fibrinogen.

In vitro cytotoxicity assessment: Cells were plated at a density between 1250 and 3333 per well of a 384-well white tissue culture treated microtiter plate in 20 μl complete media, then grown for 24 hour at 37° C. with 7% CO, in a humidified incubator before conjugate or small molecule agent addition. Cells were incubated in the presence of test compound for 72 hr prior to cell viability testing. Cell viability testing was performed using the CellTiter-Glo luminescent cell viability assay (Promega, Madison, Wis). EC50 values of the test compounds for each cell line were determined using GraphPad Prism 5 software.

### Example 2

### EDC2-EDCs that target CD147 and the Na,K-ATPase

EDC2 immunoconjugates comprise an antibody that targets cluster of differentiation 147 (CD147), a protein that in humans is encoded by the BSG gene, which codes for the expression of basigin (BSG), also known as extracellular matrix metalloproteinase inducer (EMMPRIN), and a drug that targets the Na,K-ATPase.

The following four anti-CD147 monoclonal antibodies were conjugated with CEN010-105 as described in Example 1 to prepare EDCs of the invention: (1) clone HIM6 (Mouse IgG1, κ), Biolegend, San Diego, Calif., Cat. No. 306206; (2) clone 1A6A8 (Mouse IgG1, κ); (3) clone 2C4 (Mouse IgG1, κ); and (4) clone 8D12 (Mouse IgG1, κ), eBioscience, San Diego, Calif., Cat. No. 14-1472.

The resulting EDCs of CEN010-105 conjugated to monoclonal antibodies HIM6, 1A6A8, 2C4, and, 8D12 were designated as EDC2.1, EDC2.2, EDC2.3 and EDC2.4, respectively, in accordance with the nomenclature EDCX.Y, where X refers to the target to which the targeting agent binds (e.g. CD147 in this example), and Y is the specific targeting agent employed (e.g. mAb clone HIM6 in one case in this example).

The cytotoxic activity of these EDCs was evaluated in vitro against a number of cancer cell lines as described in Example 1, and the results are summarized in Table 1 (EC50 values are in nM) in Example 8. The results show that CD147 is expressed in 9 cell lines tested and that EDCs targeting this cell surface protein are generally active in the picomolar range. These results demonstrate that the cell surface protein CD147 consistently complexes with the Na,K-ATPase in the 9 cancer cell lines tested. Therefore EDCs that employ targeting moieties to CD147 conjugated to agents that act on the Na,K-ATPase are generally useful in treating many types of cancers, including the cancer types tested as reported in Example 8 (large cell lung, colon, non-small-cell lung, pancreatic, breast, head and neck, small cell lung, melanomas and myelomas).

### Example 3

### EDC3-EDCs that target CD44 and the Na,K-ATPase

EDC3 immunoconjugates comprise an antibody that targets cluster of differentiation 44 (CD44), an 80-95 kD glycoprotein that in humans is encoded by the CD44 gene and is also known as Hermes, Pgpl, H-CAM, and HUTCH, and a drug that targets the Na,K-ATPase.

The following two anti-CD44 monoclonal antibodies were conjugated with CEN010-105 as described in Example 1 to prepare EDCs of the invention: (1) clone IM7 (Rat IgG2b, κ), Biolegend, San Diego, Calif., Cat. No. 103002; and (2) clone BJ18 (Mouse IgG1, κ), Biolegend, San Diego, Calif., Cat. No. 338802. The resulting EDCs of CEN010-105 and monoclonal antibodies 1M7 and BJ18 were designated EDC3.1 and EDC3.2 (respectively).

The cytotoxic activity of these EDCs was evaluated in vitro against a number of cancer cell lines as described in Example 1, and the results are summarized in Table I (EC50 values are in nM) in Example 8. The results show that CD44 is expressed in all the cell lines tested and that the EDCs targeting this cell surface protein are active in the low-nanomolar range for cell lines A549 and PANC1 and below 100 nM for cell lines HT29, MB231, FaDu, H46. EDC3.1 and EDC3.2 displayed activities below that of the control conjugates for all cell types. These results show that CD44 is complexed with the Na,K-ATPase in cell lines A549 and PANC1 and CD44 is either expressed at low levels or weakly (not always) complexed with the Na,K-ATPase in cell lines HT29, MB231, FaDu, H46 when cultured under the described conditions. Strong interactions are those where the sensitivity of the cell type to the EDC is at least 100-fold better than the control conjugate. Weak interactions are those where the sensitivity of the cell type to the EDC is at least 10-fold better than the control conjugate but not better than 100-fold. No interactions are those where the sensitivity of the cell type to the EDC is not at least 10-fold better than the control conjugate. Therefore, EDCs that employ targeting moieties to CD44 conjugated to agents that act on the Na,K-ATPase are generally useful in treating many types of cancers, including the cancer types tested as described in Example 8 (non-small-cell lung, and pancreatic).

### Example 4

### EDC4-EDCs that Target CD98 and the Na,K-ATPase

EDC4 immunoconjugates comprise an antibody that targets cluster of differentiation 98 (CD98) a heterodimeric glycoprotein composed of SLC3A2 and SLC7A5 that together form the large neutral amino acid transporter (LAT1), and a drug that targets the Na,K-ATPase. The following anti-CD98 antibody was conjugated with CEN010-105 as described in Example 1 to prepare an EDC of the invention: clone MEM-108 (Mouse IgG1, κ), Biolegend, San Diego, Calif., Cat. No. 315602. The resulting conjugate was designated as EDC4.1.

The cytotoxic activity of this EDC was evaluated in vitro against a number of cancer cell lines as described in Example 1, and the results are summarized in Table 1 (EC50 values are in nM) in Example 8. The results show that CD98 is expressed in all the cell lines tested under the defined conditions and that the EDC targeting this cell surface protein is active in the nanomolar range of the cell lines tested and at levels below the control conjugate, indicating that CD98 is complexed with the Na,K-ATPase in those cell lines. Therefore EDCs that employ targeting moieties to CD98 conjugated to agents that act on the Na,K-ATPase are generally useful in treating many types of cancers, including the cancer types tested as reported in Example 8 (non-small-cell lung, head and neck, small cell lung and myelom as).

### Example 5

### EDC5-EDCs that Target CD87 and the Na,K-ATPase

EDC5 immunoconjugates comprise an antibody that targets cluster of differentiation 87 (CD87), also known as the urokinase receptor or uPAR and a drug that targets the Na,K-ATPase. The following anti-CD87 antibody was conjugated to CEN010-105 as described in Example 1 to prepare an EDC of the invention: clone VIM5 (Mouse IgG1, κ), Biolegend, San Diego, Calif., Cat. No. 336902. The resulting conjugate was designated as EDC5.1.

The cytotoxic activity of this EDC was evaluated in vitro against a number of cancer cell lines as described in Example 1, and the results are summarized in Table 1 (EC50 values are in nM) in Example 8. The results show that under the conditions used to grow the cells, CD87 is expressed only on LOX cells and that EDC5.1 targeting CD87 and the Na,K-ATPase is active in the low-nanomolar range on that cell line, indicating that CD98 is complexed with the Na,K-ATPase in that cell line.

No CD87 stimulating factors were used in generating the results described in Example 8, and CD87 is not usually expressed at detectable levels on quiescent cells. CD87 therefore may require upregulation before activities of the uPAR system are initiated. For example, CD87 expression is stimulated in vitro by agents such as phorbol esters (Lund et al., J. Biol. Chem. 1991, 266:5177-5181), the transformation of epithelial cells and various growth factors and cytokines such as VEGF, bFGF, HGF, IL-1, TNF.alpha (in endothelial cells), and GM-CSF (in macrophages) (Mignatti et al., J. Cell Biol. 1991, 113:1193-1201; Mandriota et al., J. Biol. Chem. 270:9709-9716; Yoshida et al., Inflammation 1996, 20:319-326). More importantly, uPAR appears to be up-regulated in vivo in most human carcinomas examined to date, specifically in the tumor cells themselves and in tumor-associated endothelial cells undergoing angiogenesis and in macrophages (Pyke et al., Cancer Res. 1993, 53:1911-15), which may participate in the induction of tumor angiogenesis (Lewis et al., J. Leukoc. Biol. 1995, 57:747-751).

Therefore EDCs that employ targeting moieties to CD87 conjugated to agents that act on the Na,K-ATPase are generally useful in treating many types of cancers, including the cancer types tested as described in Example 8 (e.g. melanoma). In addition, EDC5.1 or EDCs developed with uPAR specific targeting moieties and agents that act on the Na,K-ATPase are useful in treating inflammatory diseases involving macrophages.

### Example 6

### EDC6-EDCs that Target CD230 and the Na,K-ATPase

EDC6 immunoconjugates comprise an antibody that targets cluster of differentiation 230 (CD230), also known as the major prion related protein (PrP), and a drug that targets the Na,K-ATPase. The following anti-CD230 antibody was coupled to CEN010-105 as described in Example 1 to prepare an EDC of the invention: clone 4D5 (Mouse IgG1, κ) eBioscience, San Diego, Calif., Cat. No. 14-9230-82. The resulting conjugate was designated as EDC6.1.

The cytotoxic activity of EDC6.1 was evaluated in vitro against a number of cancer cell lines as described in Example 1, and the results are summarized in Table 1 (EC50 values are in nM) in Example 8. The results show that CD230 is expressed in all the cell lines tested and that the EDC targeting this cell surface protein is active in the nanomolar range and below control conjugate levels on cell lines A549, and LOX, indicating that CD230 is complexed with the Na,K-ATPase in these cell lines. The results also show that EDC6.1 is not active on PANCI, MB231, or FaDu cells, indicating that CD230 is not complexed with the Na,K-ATPase on the surface of those cells under the conditions tested. Therefore, EDCs that employ targeting moieties to CD230 conjugated to agents that act on the Na,K-ATPase should generally be useful in treating many types of cancers, including cancer types tested as described in Example 8 (e.g. non-small-cell-lung and melanoma). In addition, EDCs developed with prion specific targeting moieties and agents that act on the Na,K-ATPase are useful in treating neurological defects caused by the accumulation of prions and prion related proteins.

### Example 7

### EDC7-EDCs that Target CD56 and the Na,K-ATPase

The EDC7 immunoconjugates comprise an antibody that targets cluster of differentiation 56 (CD56), also known as the neural cell adhesion molecule (NCAM) and a drug that targets the Na,K-ATPase.

The following two anti-CD56 antibodies were coupled to CEN010-105 substantially as described in Example 1 to prepare EDCs of the invention: (I) clone HCD56 (Mouse IgGI1, κ), Biolegend, San Diego, Calif., Cat. No. 318324; and (2) clone MEM-188 (Mouse IgG2a, κ), Biolegend, San Diego, Calif., Cat. No. 304622. The resulting EDCs of CEN010-105 conjugated to antibodies HCD56 and MEM-188 were designated EDC6.1 and EDC6.2, respectively.

The cytotoxic activity of these EDCs was evaluked in vitro against a number of cancer cell lines as described in Example 1, and the results are summarized in Table 1 (EC50 values are in nM) in Example 8. The results show that CD56 is expressed on only the small cell lung cancer line cells designated H69 cells and that the EDCs targeting this cell surface protein are active in the picomolar range on that cell line, indicating that CD56 is complexed with the Na,K-ATPase in that cell line. Therefore, EDCs that employ targeting moieties to CD56 conjugated to agents that act on the Na,K-ATPase should be generally useful in treating small cell lung cancers.

### Example 8

### In vitro Cytotoxicity of EDCs

TABLE 1

| | Drugs | | | | | | | | | |
|---|---|---|---|---|---|---|---|---|---|---|
| | attached | | | | | | FaDu | | | |
| | per | H460 | HT29 | A549 | PANC1 | MB231 | Head/ | H69 | LOX | H929 |
| | Antibody | LCLC | Colon | NSCLC | Pancreas | Breast | neck | SCLC | Melanoma | Myeloma |
| CD147 (EMMPRIN) | | + | + | + | + | + | + | + | + | + |
| EDC2.1 | 1 | | | 11.58 | 0.45 | | | | | |
| EDC2.2 | 7 | 0.14 | 0.28 | 0.20 | 0.15 | 0.45 | 0.19 | 0.08 | 0.20 | 0.10 |
| EDC2.3 | 6 | | | 0.26 | 0.16 | | | | | |
| EDC2.4 | 8 | 0.14 | 0.36 | 0.20 | 0.07 | 0.68 | 0.07 | 0.04 | | |
| CD44 (HCELL) | | | + | + | + | + | + | + | | |
| EDC3.1 | 5 | | 34.8 | 1.3 | 1.4 | 62.3 | 53.2 | | | |
| EDC3.2 | 3 | | | 20.3 | 2.2 | | | 34.6 | | |
| CD98 (LAT1) | | | | + | + | + | + | + | | + |
| EDC4.1 | 3 | | | 1.9 | 15.6 | >100 | 4.1 | 5.9 | | 23.7 |
| CD87 (uPAR) | | − | − | − | − | − | − | − | + | − |
| EDC5.1 | 3 | 87.6 | 111 | 43 | 68 | >200 | 140 | 42 | 3.2 | 56.5 |
| CD230 (PRP) | | | | + | + | + | + | | + | |
| EDC6.1 | 7 | | | 7.1 | 62.7 | >200 | 160.2 | | 21.4 | |
| CD56 (NCAM) | | − | − | − | − | − | − | + | − | + |
| EDC7.1 | 5 | >200 | >200 | 103.9 | 108.5 | >200 | 195.2 | 0.26 | >200 | >50 |
| EDC7.2 | 7 | 161.5 | 149.9 | 58.0 | 63.1 | >200 | 114.6 | 0.08 | >200 | >50 |
| CEN09-106 | | 1.4 | 2.9 | 0.66 | 1.1 | 3.3 | 3.1 | 1.0 | 2.5 | 3.1 |
| CEN010-105 | | 76.9 | 60.0 | 31.3 | 27.1 | 108.8 | 75.5 | 26.6 | 71.0 | 89.6 |
| Control Conjugate | 7 | 142.2 | 152.9 | 85.0 | 78.3 | 500.0 | 139.2 | 81.9 | 203.1 | 266.2 |

(EC50 values are in nM)

### Example 9

### Efficacy of EDCs in Animal Models

The efficacy of EDC2.2 (see Example 2) was demonstrated in an HT-29 human colon cancer xenograft model. Briefly, to establish a human colorectal adenocarcinoma disease model, tumors from HT-29 xenograft bearing mice were minced and 8 mm³ fragments were transplanted s.c. by trocar needle into the left flank of Hsd:Athymic Nude-Foxn1^{nu} mice (Harlan, Indianapolis, Ind.). Therapy with EDC2.2 was then initiated when the tumor volume in groups of 7 animals averaged ˜100 mm³. Treatment using vehicle control and either 1 or 5 mg/kg EDC2.2 was administered i.v. using the schedule of one injection every 3 days with 4 total injected doses (q3d×4). 15 mg/kg paclitaxel dosed at qd×5 served as a positive control treatment group. Tumor volumes were measured for each group using calibrated vernier calipers and plotted against first day of tumor implant for 69 days post-implant and 41 days post-initial dose. At day 41 post-initial dose, EDC2.2 at 1 and 5 mg/kg produced 59% and 66% growth inhibition of the tumor, respectively, when compared to vehicle. Paclitaxel at its optimum dosing produced 85% growth inhibition of the tumor when compared to vehicle. Therefore, EDCs that employ targeting moieties to CD147 conjugated to agents that act on the Na,K-ATPase should be generally useful in treating colon cancers.

1-26. (canceled)

27. An extracellular-targeted drug conjugate (EDC) comprising a targeting moiety linked by a non-cleavable linker to a therapeutic agent, wherein the targeting moiety is selected from the group consisting of an antibody, epitope binding peptide, and aptamer and binds to an extracellular target that is not a Na,K-ATPase, and wherein the therapeutic agent acts on the Na,K-ATPase.

28. The EDC of claim 27, wherein the targeting moiety is an antibody.

29. The EDC of claim 28, wherein the extracelluar target is selected from the group consisting of: CD147, LAT1, ASCT2, CD98, PrP, EpCAM, MCT1, Integrin, CD166, CD44 (HCELL), CD71, CD56, CD87, TfR1, Sel-1, IGFR, c-MET, FGFR, PDGFR, GluR2, Serotonin transporter, 5-HT1A Receptor, GABAA receptor, EAAT, TLR4, T-cell receptor, mTNF alpha (transmembrane), PLA2, RANKL, Insulin Receptor, PE-NMT, angiotensin II receptor, ATP-sensitive K channel, PE-NMT, angiotensin receptor, TNF-alpha, InsP3R, RS1, and α-klotho.

30. The EDC of claim 29, in which the antibody specifically binds CD147.

31. The EDC of claim 29, in which the antibody specifically binds CD56.

32. The EDC of claim 29, in which the antibody specifically binds CD44.

33. The EDC of claim 29, in which the antibody specifically binds CD87.

34. The EDC of claim 29, in which the antibody specifically binds CD98.

35. The EDC of claim 29, in which the antibody specifically binds CD230.

36. The EDC of claim 27, in which the therapeutic agent is selected from the group consisting of a steroid and scillarenin

37. The EDC of claim 29, wherein, on average, therapeutic agent loading per antibody is about 1 to about 6.

38. A pharmaceutical composition comprising a pharmaceutically acceptable vehicle, vector, diluent, and/or excipient and a therapeutically effective amount of an EDC of claim 27.

39. A method for treating a disease, comprising administering to a subject in need of treatment for said disease a therapeutically effective amount of an EDC of claim 27.

40. The method of claim 39, wherein the disease is selected from the group of viral infections, cancers, metastases, cellular apoptosis disorders, degenerative diseases, tissue ischemia, infectious diseases or a viral, bacterial or fungal nature, immune-mediated diseases, inflammation disorders and pathological neo-angiogenesis.

41. A method for determining if a protein is associated with a Na,K-ATPase on a cell surface, said method comprising: contacting a cell with an EDC of claim 27; and determining if the EDC has an effect on the cell that is different from an effect of contacting the cell with the targeting moiety or the therapeutic agent, wherein the protein is complexed with Na,K-ATPase where the EDC has an effect on the cell that is different from the effect of the targeting moiety or the therapeutic agent.
